SEQUENCE LISTING (43 sequences)

SEQ ID NO: 1 - DNA, 13 bp, Homo sapiens
agttcttggaagc

SEQ ID NO: 2 - DNA, 14 bp, Homo sapiens
agttttctggaagt

SEQ ID NO: 3 - DNA, 473 bp, Artificial sequence
  Synthetic sequence The nucleic acid sequence of the heavy chain of the variable region of monoclonal antibody 223
gggaattcatggaatggagctgggttattctcttcctcctgtcaataattgcaggtgtccattgccaggtccagctgcagcagtctggacctgagctggtgaagcctggggcttcagtgaggatatcctgcaaggcttctggctacaccttcacaagctactatatacactgggtgaagcagaggcctggacagggacttgagtggattggatggatttatcctggaaatgttaatactgagtacaatgagaagttcaggggcaaggccacactgactgcagacaaatcctccagcacagcctacatgcagctcagcagcctgacctctgaggactctgcggtctatttctgtgcaagtgaggagaggaattacccctggtttgcttactggggccaagggactctggtcactgtctctgcagccaaaacgacacccccacccgtctatcccttggtccctggaagcttggga

SEQ ID NO: 4 - protein, 157 aa, Artificial sequence
  Synthetic sequence The amino acid sequence of the heavy chain of the variable region of monoclonal antibody 223
EFMEWSWVILFLLSIIAGVHCQVQLQQSGPELVKPGASVRISCKASGYTFTSYYIHWVKQRPGQGLEWIGWIYPGNVNTEYNEKFRGKATLTADKSSSTAYMQLSSLTSEDSAVYFCASEERNYPWFAYWGQGTLVTVSAAKTTPPPVYPLVPGSLG

SEQ ID NO: 5 - DNA, 456 bp, Artificial sequence
  Synthetic sequence The nucleic acid sequence of the light chain of the variable region of monoclonal antibody 223
tgggaattcatggagacagacacactcctgctatgggtgctgctgctctgggttccaggctccactggtgacattgtgctgacccaatctccagcttctttggctgtgtctctagggcagagggccaccatctcctgcaaggccagccaaagtgttgaagatgatggtgaaaattatatgaactggtaccaacagaaaccaggacagtcacccaaactcctcatctatgctgcatccaatctagaatctgggatcccagccaggttcagtggcagtgggtctgggacagacttcaccctcaacatccatcctgtggaggaggaggatgctgcaacctattactgtcagcaaagtaatgaggatccattcacgttcggctcggggacaaagttggaaataaaacgggctgatgctgcaccaactgtatccatcttcccaccatccagtaagcttggg

SEQ ID NO: 6 - protein, 152 aa, Artificial sequence
  Synthetic sequence The amino acid sequence of the light chain of the variable region of monoclonal antibody 223
WEFMETDTLLLWVLLLWVPGSTGDIVLTQSPASLAVSLGQRATISCKASQSVEDDGENYMNWYQQKPGQSPKLLIYAASNLESGIPARFSGSGSGTDFTLNIHPVEEEDAATYYCQQSNEDPFTFGSGTKLEIKRADAAPTVSIFPPSSKLG

SEQ ID NO: 7 - protein, 173 aa, Homo sapiens
MARMNRPAPVEVSYKHMRFLITHNPTNATLSTFIEDLKKYGATTVVRVCEVTYDKTPLEKDGITVVDWPFDDGAPPPGKVVEDWLSLVKAKFCEAPGSCVAVHCVAGLGRAPVLVALALIESGMKYEDAIQFIRQKRRGAINSKQLTYLEKYRPKQRLRFKDPHTHKTRCCVM

SEQ ID NO: 8 - DNA, 1006 bp, Homo sapiens
aagagttgggttttcttttttaattatccaaacagtgggcagcttcctcccccacacccaagtatttgcacaatatttgtgcggggtatgggggtgggtttttaaatctcgtttctcttggacaagcacagggatctcgttctcctcattttttgggggtgtgtggggacttctcaggtcgtgtccccagccttctctgcagtcccttctgccctgccgggcccgtcgggaggcgccatggctcggatgaaccgcccggccccggtggaggtgagctacaaacacatgcgcttcctcatcacccacaaccccaccaacgccacgctcagcaccttcattgaggacctgaagaagtacggggctaccactgtggtgcgtgtgtgtgaagtgacctatgacaaaacgccgctggagaaggatggcatcaccgttgtggactggccgtttgacgatggggcgcccccgcctggcaaggtagtggaagactggctgagcctggtgaaggccaagttctgtgaggcccccggcagctgcgtggctgtgcactgcgtggcgggcctggggcgggctccagtccttgtggcgctggcgcttattgagagcgggatgaagtacgaggacgccatccagttcatccgccagaagcgccgcggacgcatcaacagcaagcagctcacctacctggagaaataccggcccaaacagaggctgcggttcaaagacccacacacgcacaagacccggtgctgcgttatgtagctcaggaccttggctgggcctggtcgtcatgtaggtcaggaccttggctggacctggaggccctgccagccctgctctgcccagcccagcagggctccaggccttggctggccccacatcgccttttcctccccgacacctccgtgcacttgtgtccgaggagcgaggagcccctcggcgccttgggtggcttctgggccctttctcctgtctccgtactccctctggcggcgctggcgtggctctg

SEQ ID NO: 9 - DNA, 447 bp, Homo sapiens
atggcccggatgaaccgcccggccccggtggaggtgagctacaaacacatgcgcttcctcatcacccacaaccccaccaacgccacgctcagcaccttcattgaggacctgaagaagtacggggctaccactgtggtgcgtgtgtgtgaagtgacctatgacaaaacgccgctggagaaggatggcatcaccgttgtggactggccgtttgacgatggggcgcccccgcccggcaaggtagtggaagactggctgagcctggtgaaggccaagttctgtgaggcccccggcagctgcgtggctgtgcactgcgtggcgggcctgggccggaagcgccgcggagccatcaacagcaagcagctcacctacctggagaaataccggcccaaacagaggctgcggttcaaagacccacacacgcacaagacccggtgctgcgttatgtag

SEQ ID NO: 10 - DNA, 2963 bp, Homo sapiens
cacaagcacatgtgtgcccacacagacccaggcatacgcaggcacacacaggcaggtgtgtgaacacaggcacatacagacaagcgggtatttgcacacatggacacacatggatgcactcaggcatgtacacacgtgtgcactcatacatgacatgcactctcacacatgcgggtgcacaccttccccgggttctctctccaccccacgtctcaccgagcaagggcttgtctctcccttgtggcttttgctgtgtggctccccgcacctgaggctgcgctggtcaggacaggttgggccatgctgcggaaacagccaggcctgaaccccaacgactcagaacagcacgggcgtttcttgcccctgttgcgtgtctgttggtgggggccctacccggaggcgtggctgagggctgcgccttccccagtgtgccggctgctgggtgaggaaacgagcacggtggagccacacggggcacctgcagctttgctgggcagagactcctcgcgctactgctgctcccacttccttggccagggccagcctgtggccacgcctgaggtcatggcaggtggggatggatgagcctccagaggcgctgggtgggcagcccccgcccctcaacgcctgtcacctcagggcctctcaggtgtggctgcagcatcctctccaggaagtcttcctgttcaccccagccaggtacatgtggtcaaggaccaggtggcggggcagggtgagcacatagcccaggtccctgctgtaaggaccaggtggcgggagcagggtgagtgtacagcccaggtttctgctgtaaggacccggtggcggggacagggtaagcgcacagcccacgtccccgctgtaaggaccaggtggcagggacagggtgagcacacagtccaggtccccgctgtaaggaccaggtggcggggacagggtgagcgcacagtccaggtccccgctgtaaggaccaggtggcggggacagggtgagcgcacagtccgggtccccgctgtaaggacccggtggcggggacagggtgagcgcacagcccgggtccccgctgtaaggacccggtggcggggacagggtgagcgcacagcccacgtccccgctgtaaggaccaggtggcggggacagggtgagcgcacagtccgggtccccgctgtaaggaccaggtggcggggacagggtgagcgcacagtccgggtccccgctgtaaggaccaggtggcggggacagggtgagcgcacagtccgggtccccgctgtaaggacccggtggcggggacagggtgagcgcacagtccgggtccccgctgtaaggacccggtggcggggacagggtgagcgcacagtccgggtccccgctgtaaggacccggtggcggggacagggtgagcgtacagcccaggtttccgctgtaaggaccaggtggcggggacagggtgagcacacagtccgggtccccgctgtaaggaccaggtggcggggacagggtgagcacacagtccgggtccccgctgtaaggaccaggtggcggggacagggtgagcgtacagcccaggtttccgctgtaaggaccaggtggcggggacagggtgagcacacagtccaggtccccgctgtaaggaccaggtggcggggacagggtgagcgcatagcccaggtccccgctgtaaggaccaggtggtggggatggtggggacagggtgagcacacagtccgggtccccgctgtaaggacccggtggcggggacagggtgagcacacagtccaggtccccgctgtaaggacccggtggcggggacagggtaagcgcacagcccacgtccccgctgtaaggaccaggtggcggggacagggtgagcgcatagcccaggtccccgctgtaaggaccaggtggtggggatggtggggacaggttgagcatgcagcccaggtccctgctgtaagggaaggtcgcgcagctgggctggaattctgggctcagcccctcccttactcacaggccccccttcctggatggtggaggtgcccaggcaccacccttgttgtctgctcatggccttggggtgggtcctaccgagctggggaagcctgggggccgtagggagcccagagtcagcctggaggaggtggcgctttggtgagtttggaaggcaagcaggggtgagctgcagggggccaggaaagggtgactgtgactctgggagcagccgtgccaaggccctgggactggaggggcttggccagcctcaaggccttactccagcccactgcactctcagattccagctccctggggcaggtgagatggccgagccaggtccttggatgatctctgttcctgttcccctcttcccaggaagcgccgcggagccatcaacagcaagcagctcacctacctggagaaataccggcccaaacagaggctgcggttcaaagacccacacacgcacaagacccggtgctgcgttatgtagctcaggaccttggctgggcctggtcgtcatgtaggtcaggaccttggctggacctggaggccctgcccagccctgctctgcccagcccagcaggggctccaggccttggctggccccacatcgccttttcctccccgacacctccgtgcacttgtgtccgaggagcgaggagcccctcgggccctgggtggcctctgggccctttctcctgtctccgccactccctctggcggcgctggccgtggctctgtctctctgaggtgggtcgggcgccctctgcccgccccctcccacaccagccaggctggtctcctctagcctgtttgttgtggggtgggggtatattttgtaaccactgggcccccagcccctcttttgcgaccccttgtcctgacctgttctcggcaccttaaattattagaccccggggcagtcaggtgctccggacacccgaaggcaataaaacaggagccgtggc

SEQ ID NO: 11 - DNA, 1321 bp, Homo sapiens
tgactatccagctctgagagacgggagtttggagttgcccgctttactttggttgggttggggggggcggcgggctgttttgttccttttcttttttaagagttgggttttcttttttaattatccaaacagtgggcagcttcctcccccacacccaagtatttgcacaatatttgtgcggggtatgggggtgggtttttaaatctcgtttctcttggacaagcacagggatctcgttctcctcattttttgggggtgtgtggggacttctcaggtcgtgtccccagccttctctgcagtcccttctgccctgccgggcccgtcgggaggcgccatggctcggatgaaccgcccggccccggtggaggtgagctacaaacacatgcgcttcctcatcacccacaaccccaccaacgccacgctcagcaccttcattgaggacctgaagaagtacggggctaccactgtggtgcgtgtgtgtgaagtgacctatgacaaaacgccgctggagaaggatggcatcaccgttgtggactggccgtttgacgatggggcgcccccgcccggcaaggtagtggaagactggctgagcctggtgaaggccaagttctgtgaggcccccggcagctgcgtggctgtgcactgcgtggcgggcctgggccggaagcgccgcggagccatcaacagcaagcagctcacctacctggagaaataccggcccaaacagaggctgcggttcaaagacccacacacgcacaagacccggtgctgcgttatgtagctcaggaccttggctgggcctggtcgtcatgtaggtcaggaccttggctggacctggaggccctgcccagccctgctctgcccagcccagcaggggctccaggccttggctggccccacatcgccttttcctccccgacacctccgtgcacttgtgtccgaggagcgaggagcccctcgggccctgggtggcctctgggccctttctcctgtctccgccactccctctggcggcgctggccgtggctctgtctctctgaggtgggtcgggcgccctctgcccgccccctcccacaccagccaggctggtctcctctagcctgtttgttgtggggtgggggtatattttgtaaccactgggcccccagcccctcttttgcgaccccttgtcctgacctgttctcggcaccttaaattattagaccccggggcagtcaggtgctccggacacccgaaggcaataaaacaggagccgtgaaaaaaaaaaaaaaaaaaaaaaaaaaaaaaaaaaaaaaaaaaaaaaaaaaaaaaaaaaaaaaaa

SEQ ID NO: 12 - DNA, 495 bp, Homo sapiens
atggtggaggtgcccaggcaccacccttgttgtctgctcatggccttggggtgggtcctaccgagctggggaagcctgggggccgtagggagcccagagtcagcctggaggaggtggcgctttggtgagtttggaaggcaagcaggggtgagctgcagggggccaggaaagggtgactgtgactctgggagcagccgtgccaaggccctgggactggaggggcttggccagcctcaaggccttactccagcccactgcactctcagattccagctccctggggcaggtgagatggccgagccaggtccttggatgatctctgttcctgttcccctcttcccaggaagcgccgcggagccatcaacagcaagcagctcacctacctggagaaataccggcccaaacagaggctgcggttcaaagacccacacacgcacaagacccggtgctgcgttatgtagctcaggaccttggctgggcctggtcgtcatgtag

SEQ ID NO: 13 - DNA, 1329 bp, Homo sapiens
tgactatccagctctgagagacgggagtttggagttgcccgctttactttggttgggttggggggggcggcgggctgttttgttccttttcttttttaagagttgggttttcttttttaattatccaaacagtgggcagcttcctcccccacacccaagtatttgcacaatatttgtgcggggtatgggggtgggtttttaaatctcgtttctcttggacaagcacagggatctcgttctcctcattttttgggggtgtgtggggacttctcaggtcgtgtccccagccttctctgcagtcccttctgccctgccgggcccgtcgggaggcgccatggctcggatgaaccgcccggccccggtggaggtgagctacaaacacatgcgcttcctcatcacccacaaccccaccaacgccacgctcagcaccttcattgaggacctgaagaagtacggggctaccactgtggtgcgtgtgtgtgaagtgacctatgacaaaacgccgctggagaaggatggcatcaccgttgtggactggccgtttgacgatggggcgcccccgcccggcaaggtagtggaagactggctgagcctggtgaaggccaagttctgtgaggcccccggcagctgcgtggctgtgcactgcgtggcgggcctgggccggaagcgccgcggagccatcaacagcaagcagctcacctacctggagaaataccggcccaaacagaggctgcggttcaaagacccacacacgcacaagacccggtgctgcgttatgtagctcaggaccttggctgggcctggtcgtcatgtaggtcaggaccttggctggacctggaggccctgcccagccctgctctgcccagcccagcaggggctccaggccttggctggccccacatcgccttttcctccccgacacctccgtgcacttgtgtccgaggagcgaggagcccctcgggccctgggtggcctctgggccctttctcctgtctccgccactccctctggcggcgctggccgtggctctgtctctctgaggtgggtcgggcgccctctgcccgccccctcccacaccagccaggctggtctcctctagcctgtttgttgtggggtgggggtatattttgtaaccactgggcccccagcccctcttttgcgaccccttgtcctgacctgttctcggcaccttaaattattagaccccggggcagtcaggtgctccggacacccgaaggcaataaaacaggagccgtgaaaaaaaaaaaaaaaaaaaaaaaaaaaaaaaaaaaaaaaaaaaaaaaaaaaaaaaaaaaaaaaaccctcggg

SEQ ID NO: 14 - DNA, 1396 bp, Homo sapiens
tgactatccagctctgagagacgggagtttggagttgcccgctttactttggttgggttggggggggcggcgggctgttttgttccttttcttttttaagagttgggttttcttttttaattatccaaacagtgggcagcttcctcccccacacccaagtatttgcacaatatttgtgcggggtatgggggtgggtttttaaatctcgtttctcttggacaagcacagggatctcgttctcctcattttttgggggtgtgtggggacttctcaggtcgtgtccccagccttctctgcagtcccttctgccctgccgggcccgtcgggaggcgccatggctcggatgaaccgcccggccccggtggaggtgagctacaaacacatgcgcttcctcatcacccacaaccccaccaacgccacgctcagcaccttcattgaggacctgaagaagtacggggctaccactgtggtgcgtgtgtgtgaagtgacctatgacaaaacgccgctggagaaggatggcatcaccgttgtggactggccgtttgacgatggggcgcccccgcccggcaaggtagtggaagactggctgagcctggtgaaggccaagttctgtgaggcccccggcagctgcgtggctgtgcactgcgtggcgggcctgggccgggctccagtccttgtggcgctggcgcttattgagagcgggatgaagtacgaggacgccatccagttcatccgccagaagcgccgcggagccatcaacagcaagcagctcacctacctggagaaataccggcccaaacagaggctgcggttcaaagacccacacacgcacaagacccggtgctgcgttatgtagctcaggaccttggctgggcctggtcgtcatgtaggtcaggaccttggctggacctggaggccctgcccagccctgctctgcccagcccagcaggggctccaggccttggctggccccacatcgccttttcctccccgacacctccgtgcacttgtgtccgaggagcgaggagcccctcgggccctgggtggcctctgggccctttctcctgtctccgccactccctctggcggcgctggccgtggctctgtctctctgaggtgggtcgggcgccctctgcccgccccctcccacaccagccaggctggtctcctctagcctgtttgttgtggggtgggggtatattttgtaaccactgggcccccagcccctcttttgcgaccccttgtcctgacctgttctcggcaccttaaattattagaccccggggcagtcaggtgctccggacacccgaaggcaataaaacaggagccgtgaaaaaaaaaaaaaaaaaaaaaaaaaaaaaaaaaaaaaaaaaaaaaaaaaaaaaaaaaaaaaaaa

SEQ ID NO: 15 - DNA, 849 bp, Homo sapiens
gtgcgcgagggcgcgcgcgtcccgagccctccacccgtcgtgccggcgccgcccggaccgccagatgctgtgtgctgtggacccacctggggttcatggagtgggccacggggcccagccctaagcactgctgcgcccagggtcgccgcgcctcctgctgaggggtccccgtgccactggctctcaccattgccctcgcctgccgatggcctctgctgcccagcctggggccagctctaccgcctgagccccctgccccactccaggactcaccgtaccccgatggggtaacgtgacacaggccccacacgtcagaggccgctgtccccacggccactgcccgtgacccctggcccaaggcagctggagttggttcagttcaagttcattcttcctctggcccttgggggcttggggcccacctctgagtgaagggggctgtctgcccatccaccaatgtggagagggcgcccccggtgtggggtccagctctggacactgcttggcggccgggttcactttgagtttttaagttttctttgctgagcttttttggttgttctttttattttttgcctctttatgactatccagctctgagagacgggagtttggagttgcccgctttactttggttgggttggggggggcggcgggctgttttgttccttttcttttttaagagttgggttttcttttttaattatccaaacagtgggcagcttcctcccccacacccaagtatttgcacaatatttgtgcggggtatgggggtgggtttttaaatctcgtttctcttggacaagcacagggatctcgttctcctcattttttgggggtgtgtgggga

SEQ ID NO: 16 - DNA, 168 bp, Homo sapiens
tgtgaggcccccggcagctgcgtggctgtgcactgcgtggcgggcctgggccgggctccagtccttgtggcgctggcccttattgagagcgggatgaagtacgaggacgccatccagttcatccgccagaagcgccgcggagccatcaacagcaagcagctcacctac

SEQ ID NO: 17 - DNA, 2291 bp, Mus musculus
gacgcgcgccggtctgagtgtttgctcccggcgggggccgcgccccgcggcagcccctgtgtgaacccggccggtggttgccacgtgcagggccggagccgcagggaggccaggagtggggactcagccagctgttttttcggtgtggcgaccagacctgatgggtgaaaagggtgactctgctcccatttctaggggtgcggaagattccctcaacatctgcccggttttcatcttccctaagagatccctatctaggatttggttcgcctcaatttccttctctgcatagcttgggggcataggaccccatatcctttcttgcctcccgcatgaatgtcagtgggtcacctttgaaggctgggcattaagtcttgggtcccttagagacccgagtcctggcttaggcactctgggtcaggccagatgggacagcacctcactgtccatggggtcctgccgtagcctgtccacatgcagccggcttaagcaggcgactcctggtagggagctgcaggcatggctggactggatgccatgcttagcccccaaggcaggcagagtgaccatccctctgaccctatcagtgaggggcagagtggattccaggccggttgtcacagtgtgctgtgtccattggattgtgccttctgaagaccagggtgggattgatggagaagccccaggagggcagctctgactactgccgttcccaacaccgtgggtgctgccgctgaggagacctgtacacctctggccctcaccattgtccttgcctcccaatggcctctgctgccaggccggaggccagcactgctgcctgagcccgctgcccctcttcaggacttgccgttccctgatggggtaacgtgacagaccggatcagaggctgcctgcccaccacggcccagggccgctagagtttggttgagttcaagttcattttccctttggtcctgacttctggggcccaactctgaccaaaggggacactcctccgtccacccatgcagagagggtgtctccagcgacggccccatagaggacactgctctgcggccggagtcaccaacccgaagttctctctgctcagttttttttggttgttgttatttttattttgactccttgtaacttctccactctgagggacaggactttggcgctgcccgtcttgctgcggggtggggggagaagtgtgcttgtttcttttcttttttagaattggtttttttggaattatccgccacgaggcagcttcctctccctctcccaggtatttgcacaatatttgtgcggggcgtaggggcgaggttttaagaagtcttttctttgtggacaagcacggggatctcactggacttggtgtggggggctgggggaccccccgtgcagcccttgctggctagtcccctctgggtccccggaggaggcatggcccgcatgaaccggcctgcgcctgtggaggtgagctaccggcacatgcgcttcctcatcacccacaaccccagcaatgccaccctcagcacgttcatcgaggacctgaagaagtacggggctaccactgtggtgcgcgtgtgtgaagtgacctatgacaagacccccctggagaaggacggcatcactgttgtggactggccctttgatgatggagcgccccctcctggcaaagtggtagaggactggctgagcctgctgaaggccaagttctacaatgacccgggaagctgcgtagctgtgcactgtgtggcgggcctgggaagggccccagtgctcgtggctctcgccctcatcgagagcgggatgaagtacgaggacgccatccagttcatccgacagaagcgccgtggggccatcaacagcaagcagctcacctacctggagaagtaccggcctaagcagagactgaggttcaaagacccacacacgcacaagaccagatgctgcgtcatgtagctcaggccctggccctgtacctcattacatctgtgtctaaggagtccaacggctatgtgcgtccctgctctgtccccatctgtacccacggctgtctttctgaagctgtccctggaccctctgccagtcctgtccaacccctgtccctcaccccccactgcccaggccttccccctggcctgtgtattgcaggtgggagtttttaaaccactgggcccaatgcctcagcggcgtggccctcaccctaacctttttccagcacctttgttaccaggtgctctggactctcaaggcaataaatcaggagctgtgaaaacaaaaaaaaaaaaaaaaaa

SEQ ID NO: 18 - DNA, 963 bp, Homo sapiens
cttttttaattatccaaacagtgggcagcttcctcccccacacccaagtatttgcacaatatttgtgcggggtatgggggtgggtttttaaatctcgtttctcttggacaagcacagggatctcgttctcctcattttttgggggtgtgtggggacttctcaggtcgtgtccccagccttctctgcagtcccttctgccctgccgggcccgtcgggaggcgccatggctcggatgaaccgcccggccccggtggaggtgagctacaaacacatgcgcttcctcatcacccacaaccccaccaacgccacgctcagcaccttcattgaggacctgaagaagtacgaggacgccatccagttcatccgccagaagcgccgcggagccatcaacagcaagcagctcacctacctggagaaataccggcccaaacagaggctgcggttcaaagacccacacacgcacaagacccggtgctgcgttatgtagctcaggaccttggctgggcctggtcgtcatgtaggtcaggaccttggctggacctggaggccctgcccagccctgctctgcccagcccagcaggggctccaggccttggctggccccacatcgccttttcctccccgacacctccgtgcacttgtgtccgaggagcgaggagcccctcgggccctgggtggcctctgggccctttcttctgtctccgccactccctctggcggcgctggccgtggctctgtctctctgaggtgggtcgggcgccctctgcccgccccctcccacaccagccaggctggtctcctctagcctgtttgttgtggggtgggggtatattttgtaaccactgggcccccagcccctcttttgcgaccccttgtcctgacctgttctcggcaccttaaattattagaccccggggcagtcaggtgctccggacacccgaaggcaataaaacaggagccgtg

SEQ ID NO: 19 - DNA, 296 bp, Mus musculus
  misc_feature (94)..(94): n is a, c, g, or t
cggcctaagcagagactgaggttcaaagacccacacacgcacaagaccagatgctgcgtcatgtagctcaggccctggccctgtacctcattanatctgtgtctaaggagtccaacggctatgtgcgtccctgctctgtccccatctgtacccacggctgtctttctgaagctgtccctggaccctctgccagtcctgtccaacccctgtccctcaccccccactgcccaggccttccccctggcctgtgtattgcaggtgggaggttttaaaccactgggcccaatgnctcagcg

SEQ ID NO: 20 - DNA, 522 bp, Mus musculus
atggcccgcatgaaccggcctgcgcctgtggaggtgagctaccggcacatgcgcttcctcatcacccacaaccccagcaatgccaccctcagcacgttcatcgaggacctgaagaagtacggggctaccactgtggtgcgcgtgcgtgaagtgacctatgacaagacccccctggagaaggacggcatcactgttgtggactggccctttgatgatggagcgccccctcctggcaaagtggtagaggactggctgagcctgctgaaggccaagttctacaatgacccgggaagctgcgtagctgtgcactgtgtggcgggcctgggaagggccccagtgctcgtggctctcgccctcatcgagagcgggatgaagtacgaggacgccatccagttcatccgacagaagcgccgtggggccatcaacagcaagcagctcacctacctggagaagtaccggcctaagcagagactgaggttcaaagacccacacacgcacaagaccagatgctgcgtcatgtag

SEQ ID NO: 21 - DNA, 6461 bp, Mus musculus
gagtctgggagtggtggctcttaaggtgaagtgtgggctagtgcggctgagcacacagtagctgttcagtgtgcgcatatgaagctggagagctggaagggttcctcctggcttgtggcccctctgcagaaggaggtctcctacccagcaggatgcagaggggatggcaggcgggtccgcggtggtcctctcccttcctccttcctcttcctctgctggccgcgcctttgtttgcgtgctatctttaggcagcaggtgcgggagccgctgtgagtcgggtcgcccgccggtcaccgcccccccacgtgacgccgggcgctataaatagctgggcggcgggcggcgggcatccgcccggagccggcgccgctgcggagggcgcgcacgggtcccggcccggccggccggcgcatggaggcggccgcacgcctgcgggcgcgggtgagccgggctgcgggcggcggggacccggcctcgaggacgctcccgaaccctaaccctactgggatgggccctgcacgggatggcggctccctggcgggctgggtgggaagtcccgctgaggtcggggagctgtgtgccctccccacgccccactacgggtccttggggtccccagcccatccacggagcagtcttgcctgtcttgacagcacagagtcctgaactgtgacccctgactcctaagtgtcacacagggtcccatttaggtcccttctgaagctagatgcactcccaccccaccacttgagggtgtggcttctgactacctctgtgtctttttttcccccctggggttcaagtgttcccgattactagactcgatgttgagaaaacctgggtgtctggacggccggagctggccagggtccagagaaggtccttccacgcccctctgtcccatgtcctcctctggcaaacagggacaaaaatgggtcttgaaaaacatgccggtctgggagcagctgagggcttaagatactgctagaggaggctgggcggggatagagaagggcccaggtcatagtagggtccttaagcaccaggggtggggagggtgtggtggcttgactgagtgcctgttgaggcaaggtgagctcagcttctgttcacacctagagctgctgggataggggtgtaggtagccggtgggcccacaagccttggctcaggcctgctagaatgcagtagaggtagacccccccacccccccaccttgttctctggtcttcttaaccccatagaagtgatgtgtccacctgggcatggtggcagatgccttaagtcctagcactcaggaggcccagccaggtggatctctgtgagtctgaggtcagcctggtctacaaagtgcggtccaggaggtaagacagggacagagaaacactgtctccaaagggggtgagcggagtgatgtgtcggcagaggggctcctttttgggagggcagccaccatgtggttgagcctgggtagactcaggcatgctgtgggcatgcccctttttgcccacacttaggggtgtggcccagactgcagtgaactgtgcatgtattataagatgcagttcagcctggtgtcacctatacatctggacccctggctccatcccctcaggcctggggggagcatgataggcggttcaggcttccttttctcagatgttgcttctgttgcccctggaatcttacaacccagccccaacccctgcaagtagccagtaggtgcagttagctctacaggagcctgccagtgttctctgactccctggggccttctcttcttttctcctgcacacacaagggcattgcagtgtgcacacatgggatgctcatctcagtaggcccgggtgtttgtgtcataacatgagagaaggctctccttgcttatagcgggctagtcgtggctccgcatgcacagggcgttgagaacctggcctgcccaagcaggcacaggactcctttctgccctcctccaaggggcccctctatcttagccctttcagaatggtggcagggactagctgtgagccatggcactgacttggagtggagtgctgagctgttctcaggtgtaggaggtagcgagggaggattctgtgctatctagtacagtcgagccttggacggtggggcctgtgcccgaagcttggggccagagaccagggtcttatgctggcctgaggctccaagggcctggtttgcttggtgggcagctggagaagggagagctggggacgagacagagaggtaagaggtacgggcagaactcttccagatgcgatccagaaggggccttccagaaagatgaatgctgggtgggggatgggctgagtccaggggctcaggctctactaactactggatcatagcccaagggactgtggtgccctctttttcaaccttcatcgtgttgatgggaaacgggtccagagaggccagtgacatgcccaaggtctcccaggccgtccagggggtggcagggtgttagctagctgttctttggggtccttgcagaggagaaggtggaggccgatgtcctccagtagtgcccttcccctgggtcctgtcaacggtgtgactcccttcctggccacctgtgctgtcacctccttggcatgagcctggcagctggccccttggcctgtggtctcttgcaaagcagaacgtcttgtttacctctagtgcctggcaagtccccgtaagttgtggctgtgggtagagatttcagaaagcctgaggggcagcaattgaggccactcagccccttgtccctctggcagtacccactgtccctgagacctgcccttgcagaacgatctgagcttaatattgtgctgtggctagcagtcccaagagagggaccactgcccttatgggctgtgagatgggggacctggcttctgctgctgtaggtgtactctgcttgtggggttcatatgctcctggggtgagccctgataccctcttcaccctatctcggtggctatgtatggctctcttgtcaccacaggggcctggggtagcccaggcaggagttgtctgtctgtttctgggtttcacgtggagaatctgaggacctcccatttctttctcccctcccatagattgtgccttctgaagaccagggtgggattgatggagaagccccaggagggcagctctgactactgccgttcccaacaccgtgggtgctgccgctgaggagacctgtacacctctggccctcaccattgtccttgcctcccaatggcctctgctgccaggccggaggccagcactgctgcctgagcccgctgcccctcttcaggacttgccgttccctgatggggtaacgtgacagaccggatcagaggctgcctgcccaccacggcccagggccgctagagtttggttgagttcaagttcattttccctttggtcctgacttctggggcccaactctgaccaaaggggacactcctccgtccacccatgcagagagggtgtctccagcgacggccccatagaggacactgctctgcggccggagtcaccaacccgaagttctctctgctcagttttttttggttgttgttatttttattttgactccttgtaacttctccactctgagggacaggactttggcgctgcccgtcttgctgcggggtggggggagaagtgtgcttgtttcttttcttttttagaattggtttttttggaattatccgccacgaggcagcttcctctccctctcccaggtatttgcacaatatttgtgcggggcgtaggggcgaggttttaagaagtcttttctttgtggacaagcacggggatctcactggacttggtgtggggggctgggggaccccccgtgcagcccttgctggctagtcccctctgggtccccggaggaggcatggcccgcatgaaccggcctgcgcctgtggaggtgagctaccggcacatgcgcttcctcatcacccacaaccccagcaatgccaccctcagcacgttcatcgaggtgagtgggccttcagggccgggtgggtgtcctgcacagctcagggtgcgatgggtgctcacgatccgatggctttcctggagggctgtcagcatccgtgcttcggggaatggacattgtctagtctgtggctgtccagaccctgggcgaccttagcctctgcagagacagagcaaataaagaggccgaggagggaggggctgacagggaaacggctcagagacaggcagattggaagaaggactgggagagctaccctgtggctaggccagcccacaagctcaggtgttaggaagccttagggaacagagagtgaccattgcagtctgtggaggtgacgtgtggagagtccacaagtgtctgtggtctgtggccaccctcacttcacccctagatgtgaccaggtatttttttctgagcctgggtgggggaatatagctgggtggggtgggggagtgctgtcagcatggcctcactggggaccctgggcagggctccttactgcagatctggagctgcttgcagagtctgcatctggatgctgggccattggtggaatggacttttgtggctttatgttcttagtccttttctctgtggcacgagctaagccccatgggctcgtgggcatcaaaaagggacgaccgcatagaggaagacaatggacacccgaagactgccgactctcagttgagaagctttgtgacagtagaggctgatgaagggctaggaaaacaaaacaagacaaaaaccaaaacaaaccaaaacaaaccaccacgagtgaccaggacaggggctcgctcggtgtgtgaccagaatgagggtttcgcaaccatagtcaaggctcactgtatcaccagggtcagggaccacatggcaaacatgatctgatcagggttcagggtgtgagcagagttgggcctgaagcctgagctggctttctccttgatggactctgcagcaccccatggtctctgaggtggaccccctccctcgttttctgctttttgttttttcttgcactctcaaaatagttgggtcttggccttgctgtgtctgttgttcctatcacatggccagccagaccgtcatccctcaccactcccaaaatatccactgggggcaggtaggtgggggccggccctgcttgtggctgtgggtataaatactccacattggctgtgagctgagtgaacttttctctggaggaatggggcttcccccgtggtatttgagtggtgccacccagatggcgctggggttccaactcctggggtctacttgctggacactcgggaaggactctgtgggacccacccatgaagctcaggaaggggtctatctgaggcctaggccaccctgccggggacctagagctttatctggagaaccaggctttgcagtcaagaccctttgtcctaggtgctgcaagggtgagagggacaggaggggcttgaatcacagcagctgagctgtgtgtggcacaggtgggacagggacaagtgaccccgaagcatcaccaggctaccttgttagtcgctaagcatccaagcccccctgttgatttatgaaatctgcttgtctcaagggaagcagcttgaggggacactgctctgtttaggagttctagagggtaggcctgtggggactcagcatgggtagaggcctgggctggaggccgttctgggttctgaactgtcccccccccatctctctctttctgtcccaggacctgaagaagtacggggctaccactgtggtgcgcgtgtgtgaagtgacctatgacaagacccccctggagaaggacggcatcactgttgtggactggccctttgatgatggagcgccccctcctggcaaagtggtagaggactggctgagcctgctgaaggccaagttctacaatgacccgggaagctgcgtagctgtgcactgtgtggcgggcctgggaagggccccagtgctcgtggctctcgccctcatcgagagcgggatgaagtacgaggacgccatccagttcatccgacagaagcgccgtggggccatcaacagcaagcagctcacctacctggagaagtaccggcctaagcagagactgaggttcaaagacccacacacgcacaagaccagatgctgcgtcatgtagctcaggccctggccctgtacctcattacatcggtgtctaaggagtccaacggctatgtgcgtccctgctctgtccccatccgtacccacggctgtctttctgaagctgtccctggaccctctgccagtcctgtccaacccctgtccctcaccccccactgcccaggccttccccctggcctgtgtattgcaggtgggagtttttaaaccactgggcccaatgcctcagcggcgtggccctcaccctaacctttttccagcacctttgttaccaggtgctctggactctcaaggcaataaatcaggagctgtg

SEQ ID NO: 22 - DNA, 1722 bp, Mus musculus
gttccgcccggagccggcgccgctgcggagggcgcgcacgggtcccggcccggccggccggcgcatggaggcggccgcacgcctgcgggcgcggattgtgccttctgaagaccagggtgggattgatggagaagccccaggagggcagctctgactactgccgttcccaacaccgtgggtgctgccgctgaggagacctgtacacctctggccctcaccattgtccttgcctcccaatggcctctgctgccaggccggaggccagcactgctgcctgagcccgctgcccctcttcaggacttgccgttccctgatggggtaacgtgacagaccggatcagaggctgcctgcccaccacggcccagggccgctagagtttggttgagttcaagttcattttccctttggtcctgacttctggggcccaactctgaccaaaggggacactcctccgtccacccatgcagagagggtgtctccagcgacggccccatagaggacactgctctgcggccggagtcaccaacccgaagttctctctgctcagttttttttggttgttgttatttttattttgactccttgtaacttctccactctgagggacaggactttggcgctgcccgtcttgctgcggggtggggggagaagtgtgcttgtttcttttcttttttagaattgggttttttggaattatccgccacgaggcagcttcctctccctctcccaggtatttgcacaatatttgtgcggggcgtaggggcgaggttttaagaagtcttttctttgtggacaagcacggggatctcactggacttggtgtggggggctgggggaccccccgtgcagcccttgctggctagtcccctctgggtccccggaggaggcatggcccgcatgaaccggcctgcgcctgtggaggtgagctaccggcacatgcgcttcctcatcacccacaaccccagcaatgccaccctcagcacgttcatcgaggacctgaagaagtacggggctaccactgtggtgcgcgtgtgtgaagtgacctatgacaagacccccctggagaaggacggcatcactgttgtggactggccctttgatgatggagcgccccctcctggcaaagtggtagaggactggctgagcctgctgaaggccaagttctacaatgacccgggaagctgcgtagctgtgcactgtgtggcgggcctgggaagggccccagtgctcgtggctctcgccctcatcgagagcgggatgaagtacgaggacgccatccagttcatccgacagaagcgccgtggggccatcaacagcaagcagctcacctacctggagaagtaccggcctaagcagagactgaggttcaaagacccacacacgcacaagaccagatgctgcgtcatgtagctcaggccctggccctgtacctcattacatctgtgtctaaggagtccaacggctatgtgcgtccctgctctgtccccatctgtacccacggctgtctttctgaagctgtccctggaccctctgccagtcctgtccaacccctgtccctcaccccccactgcccaggccttccccctggcctgtgtattgcaggtgggagtttttaaaccactgggcccaatgcctcagcggcgtggccctcaccctaacctttttccagcacctttgttaccaggtgctctggactctcaaggcaataaatcaggagctgtg

SEQ ID NO: 23 - DNA, 1720 bp, Mus musculus
tccgcccggagccggcgccgctgcggagggcgcgcacgggtcccggcccggccggccggcgcatggaggcggccgcacgcctgcgggcgcggattgtgccttctgaagaccagggtgggattgatggagaagccccaggagggcagctctgactactgccgttcccaacaccgtgggtgctgccgctgaggagacctgtacacctctggccctcaccattgtccttgcctcccaatggcctctgctgccaggccggaggccagcactgctgcctgagcccgctgcccctcttcaggacttgccgttccctgatggggtaacgtgacagaccggatcagaggctgcctgcccaccacggcccagggccgctagagtttggttgagttcaagttcattttccctttggtcctgacttctggggcccaactctgaccaaaggggacactcctccgtccacccatgcagagagggtgtctccagcgacggccccatagaggacactgctctgcggccggagtcaccaacccgaagttctctctgctcagttttttttggttgttgttatttttattttgactccttgtaacttctccactctgagggacaggactttggcgctgcccgtcttgctgcggggtggggggagaagtgtgcttgtttcttttcttttttagaattggtttttttggaattatccgccacgaggcagcttcctctccctctcccaggtatttgcacaatatttgtgcggggcgtaggggcgaggttttaagaagtcttttctttgtggacaagcacggggatctcactggacttggtgtggggggctgggggaccccccgtgcagcccttgctggctagtcccctctgggtccccggaggaggcatggcccgcatgaaccggcctgcgcctgtggaggtgagctaccggcacatgcgcttcctcatcacccacaaccccagcaatgccaccctcagcacgttcatcgaggacctgaagaagtacggggctaccactgtggtgcgcgtgtgtgaagtgacctatgacaagacccccctggagaaggacggcatcactgttgtggactggccctttgatgatggagcgccccctcctggcaaagtggtagaggactggctgagcctgctgaaggccaagttctacaatgacccgggaagctgcgtagctgtgcactgtgtggcgggcctgggaagggccccagtgctcgtggctctcgccctcatcgagagcgggatgaagtacgaggacgccatccagttcatccgacagaagcgccgtggggccatcaacagcaagcagctcacctacctggagaagtaccggcctaagcagagactgaggttcaaagacccacacacgcacaagaccagatgctgcgtcatgtagctcaggccctggccctgtacctcattacatctgtgtctaaggagtccaacggctatgtgcgtccctgctctgtccccatctgtacccacggctgtctttctgaagctgtccctggaccctctgccagtcctgtccaacccctgtccctcaccccccactgcccaggccttccccctggcctgtgtattgcaggtgggagtttttaaaccactgggcccaatgcctcagcggcgtggccctcaccctaacctttttccagcacctttgttaccaggtgctctggactctcaaggcaataaatcaggagctgtg

SEQ ID NO: 24 - DNA, 817 bp, Mus musculus
tagaattggtttttttggaattatccgccacgaggcagcttcctctccctctcccaggtatttgcacaatatttgtgcggggcgtaggggcgaggttttaagaagtcttttctttgtggacaagcacggggatctcactggacttggtgtggggggctgggggaccccccgtgcagcccttgctggctagtcccctctgggtccccggaggaggcatggcccgcatgaaccggcctgcgcctgtggaggtgagctaccggcacatgcgcttcctcatcacccacaaccccagcaatgccaccctcagcacgttcatcgaggacctgaagaagtacggggctaccactgtggtgcgcgtgtgtgaagtgacctatgacaagacccccctggagaaggacggcatcactgttgtggactggccctttgatgatggagcgccccctcctggcaaagtggtagaggactggctgagcctgctgaaggccaagttctacaatgacccgggaagctgcgtacttgtgcactgtgtggcgggcctgggaagggccccagtgctcgtggctctcgccctcatcgagagcgggatgaagtacgaggacgccatccagttcatccgacagaagcgccgtggggccatcaacagcaagcagctcacctacctggagaagtaccggcctaagcagagactgaggttcaaagacccacacacgcacaagaccagatgctgcgtcatgtagctcaggccctggccgtgtacctcattacatctgtgtctaaggagtccacggctatgtgcgtcctgctctgtcccatctgt

SEQ ID NO: 25 - DNA, 3222 bp, Mus musculus
ggtcccggcccggccggcctggcgcatggaggcggccgcacgcctgcgggcgcggattgtgccttctgaagaccagggtgggattgatggagaagccccaggagggcagctctgactactgccgttcccaacaccgtgggtgctgccgctgaggagacctgtacacctctggccctcaccattgtccttgcctcccaatggcctctgctgccaggccggaggcagcactgctgcctgagcccgctgcccctcttcaggacttgccgttccctgatggggtaacgtgacagaccggatcagaggctgcctgcccaccacggcccagggccgctagagtttggttgagttcaagttcattttccctttggtcctgacttctggggcccaactctgaccaaaggggacactcctccgtccacccatgcagagagggtgtctccagcgacggccccatagaggacactgctctgcggccggagtcaccaacccgaagttctctctgctcagttttttttggttgttgttatttttattttgactccttgtaacttctccactctgagggacaggactttggcgctgcccgtcttgctgcggggtggggggagaagtgtgcttgtttcttttcttttttagaattggtttttttggaattatccgccacgaggcagcttcctctccctctcccaggtatttgcacaatatttgtgcggggcgtaggggcgaggttttaagaagtcttttctttgtggacaagcacggggatctcactggacttggtgtggggggctgggggaccccccgtgcagcccttgctggctagtcccctctgggtccccggaggaggcatggcccgcatgaaccggcctgcgcctgtggaggtgagctaccggcacatgcgcttcctcatcacccacaaccccagcaatgccaccctcagcacgttcatcgaggacctgaagaagtacggggctaccactgtggtgcgcgtgtgtgaagtgacctatgacaagacccccctggagaaggacggcatcactgttgtggactggccctttgatgatggagcgccccctcctggcaaagtggtagaggactggctgagcctgctgaaggccaagttctacaatgacccgggaagctgcgtagctgtgcactgtgtggcgggcctgggaagggccccagtgctcgtggctctcgccctcatcgagagcgggatgaagtacgaggacgccatccagttcatccgacagaagcgccgtggggccatcaacagcaagcagctcacctacctggagaagtaccggcctaagcagagactgaggttcaaagacccacacacgcacaagaccagatgctgcgtcatgtagctcaggccctggccctgtacctcattacatctgtgtctaaggagtccaacggctatgtgcgtccctgctctgtccccatctgtacccacggctgtctttctgaagctgtccctggaccctctgccagtcctgtccaacccctgtccctcaccccccactgcccaggccttccccctggcctgtgtattgcaggtgggagtttttaaaccactgggcccaatgcctcagcggcgtggccctcaccctaacctttttccagcacctttgttaccaggtgctctggactctcaaggcaataaatcaggagctgtggatgtgtgtgaagagtgttatagagcaggggaatagggtgtggatcccgggaggccatggtctccatctcttctgtcctattgctgctgctgctactgctgctgcagtggcgtgcctccatggcgccctctggtggccatcccttgctctgcctccctgacttgttctacaagttcttgacgcaaaaatcagcacagcttggactggtgacggtttgtaacattaggaggtgagactgatcacctctgcaggacagagtagggggtcacttggaactttcagtggcctccacccccgaccttcatgcaaccagaggtgtgggttgcaggtagatctgagtgtagatggcctttggtgacatgggtctgcctaggacctcatcctgcttataagttctgagcagtgggctgactcttcccttagctgaggaaagggtatcatgagggacagggctggctatatgtgtgtcttagccagggttttatggctgtgaacagacaccgggaccaaggcaactcttacaaggacaacatttagttggggctgccttacaggttcagaggttcagtctgtcatcaaggtgggagcatggcagcatccatgcaggcatggggcagctgtagctgagagctctacaccttcatctgaaggctactagtggaagactgatttccagggagctagaatgagggccttaaagcccatgcccacagtgacacacctactccaacaaggtcagacctccaaacggtgccactccccaggccagtcgtatttaaacagtgacaatgtgtcagtgtggatgtgctggctgtcttatagcactgacatacttaggacgtgatcctcaggcctttgcagacccagtcccctctgtgcccagaacagagatccagggcgcctctcactggtacctgctgcctgcttgcatcattccctcttcaagttccacctagctctggactaggtcccagaaaccatccgggcccacgtagactcccagcagtccctgttcttgcctgtcctagtagggagtgacaaggtgtagccaaactcagtaatggtgaccttgtgtgggctggaaactcactaccccggtgccatatttccacaaagtcactggtttttgtttttgttttagtctgccaagctttttttttttttaaaagatttatttattttatgtatatgaatatactgtagctgtacagatggttgtgagccttcatgtggttgtcgggaattgaatttaggacctctgcttgctctggtcaactccgctcactccggttcgccaatctctctcagtccttgctagcttcggcccaaagatttatttattatacataagtacactgttgctgtcttcagacataccagaagaggacgccagatctcattaccggtggttgtgagccaccatgtggttgctgagatttgaactcaggactttcggaagagccatctgaccagccccaagcttttttatttttattttttaaatctttaaatttaaaaaaaattattatttgtttgctgt

SEQ ID NO: 26 - DNA, 3222 bp, Mus musculus; identical to SEQ ID NO: 25

SEQ ID NO: 27 - DNA, 1678 bp, Mus musculus
  misc_feature (220)..(220): n is a, c, g, or t
ggcgcgtgccgagccctccgcgtcgtgccggcgccggcgcccggaccgccagattgtgccttctgaagaccagggtgggattgatggagaagccccaagagggcagctctgactactgccgttcccaacaccgtgggtgctgccgctgaggagacctgtacacctctggccctcaccattgtccttgcctcccaatggcctctgctgccaggccggagcngncactgctgcctgagcccgctgcccctcttcaggacttgccgttccctgatggggtaacgtgacagaccggatcagaggctgcctgcccaccacggcccagggccgctagagtttggttgagttcaagttcattttccctttggtcctgacttctggggcccaactctgaccaaaggggacactcctccgtccacccatgcagagagggtgtctccagcgacggccccatagaggacactgctctgcggccggagtcaccaacccgaagttctctctgctcagttttttttttggttgttgttatttttattttgactccttgtaacttctccactctgagggacaggactttggcgctgcccgtcttgctgcggggtggggggagaagtgtgcttgtttcttttcttttttagaattggtttttttggaattatccgccacgaggcagcttcctctccctctcccaggtatttgcacaatatttgtgcggggcgtaggggcgaggttttaagaagtcttttctttgtggacaagcacggggatctcactggacttggtgtgtggggctgggggacccccgtgcagccttgctggctagtccctctgggtccccggaggaggcatggcccgcatgaaccggcctgcgcctgtggaggtgagctaccggcacatgcgcttcctcatcacccacaaccccagcaatgccaccctcagcacgttcatcgaggacctgaagaagtacggggctaccactgtggtgcgcgtgtgtgaagtgacctatgacaagacccccctggagaaggacggcatcactgttgtggactggccctttgatgatggagcgccccctcctggcaaagtggtagaggactggctgagcctgctgaaggccaagttctacaatgacccgggaagctgcgtagctgtgcactgtgtggcgggcctgggaagggccccagtgctcgtgctctcgccctcatcgagagcgggatgaagtacgaggacgccatccagttcatccgacagaagcgccgtggggccatcaacagcaagcagctcacctacctggagaagtaccggcctaagcagagactgaggttcaaagacccacacacgcacaagaccagatgctgcgtcatgtagctcaggccctggccctgtacctcattacatctgtgtctaaggagtccaacggctatgtgcgtccctgctctgtccccatctgtacccacggctgtctttctgaagctgtccctggaccctctgccagtcctgtccaacccctgtccctcaccccccactgcccaggccttccccctggcctgtgtattgcaggtgggagtttttaaaccactgggcccaatgcctcagcggcgtggccctcaccctaacctttttccagcacctttgttaccaggtgctctggactctcaaggcaataaatcaggagctgtgg

SEQ ID NO: 28 - DNA, 806 bp, Mus musculus
tggaggtgagctaccggcacatgcgcttcctcatcacccacaaccccagcaatgccaccctcagcacgttcatcgaggacctgaagaagtacggggctaccactgtggtgcgcgtgtgtgaagtgacctatgacaagacccccctggagaaggacggcatcactgttgtggactggccctttgatgatggagcgccccctcctggcaaagtggtagaggactggctgagcctgctgaaggccaagttctacaatgacccgggaagctgcgtagctgtgcactgtgtggcgggcctgggaagggccccagtgctcgtggctctcgccctcatcgagagcgggatgaagtacgaggacgccatccagttcatccgacagaagcgccgtggggccatcaacagcaagcagctcacctacctggagaagtaccggcctaagcagagactgaggttcaaagacccacacacgcacaagaccagatgctgcgtcatgtagctcaggccctggccctgtacctcattacatctgtgtctaaggagtccaacggctatgtgcgtccctgctctgtccccatctgtacccacggctgtctttctgaagctgtccctggaccctctgccagtcctgttcaacccctgtccctcaccccccactgcccaggccttccccctggcctgtgtattgcaggtgggagtttttaaaccactgggcccaatgcctcagcggcgtggccctcaccctaacctttttccagcacctttgttaccaggtgctctggactctcaaggcaataaatcaggagctgcg

SEQ ID NO: 29 - DNA, 899 bp, Mus musculus
gtgcagcccttgctggctagtcccctctgggtccccggaggaggcatggcccgcatgaaccggcctgcgcctgtggaggtgagctaccggcacatgcgcttcctcatcacccacaaccccagcaatgccaccctcagcacgttcatcgaggacctgaagaagtacggggctaccactgtggtgcgcgtgtgtgaagtgacctatgacaagacccccctggagaaggacggcatcactgttgtggactggccctttgatgatggagcgccccctcctggcaaagtggtagaggactggctgagcctgctgaaggccaagttctacaatgacccgggaagctgcgtagctgtgcactgtgtggcgggcctgggaagggccccagtgctcgtggctctcgccctcatcgagagcgggatgaagtacgaggacgccatccagttcatccgacagaagcgccgtggggccatcaacagcaagcagctcacctacctggagaagtaccggcctaagcagagactgaggttcaaagacccacacacgcacaagaccagatgctgcgtcatgtagctcaggccctggccctgtacctcattacatctgtgtctaaggagtccaacggctatgtgcgtccctgctctgtccccatctgtacccacggctgtctttctgaagctgtccctggaccctctgccagtcctgtccaacccctgtccctcaccccccactgcccaggccttccccctggcctgtgtattgcaggtgggagtttttaaaccactgggcccaatgcctcagcggcgtggccctcaccctaacctttttccagcacctttgttaccaggtgctctggactctcaaggcaataaatcaggagctgtgaaaaaaaaaaaaaaaaaaaa

SEQ ID NO: 30 - DNA, 3422 bp, Homo sapiens
atgccggcgttggcgcgcgacggcggccagctgccgctgctcgttgttttttctgcaatgatatttgggactattacaaatcaagatctgcctgtgatcaagtgtgttttaatcaatcataagaacaatgattcatcagtggggaagtcatcatcatatcccatggtatcagaatccccggaagacctcgggtgtgcgttgagaccccagagctcagggacagtgtacgaacgtgccgctgtggaagtggatgtatctgcttccatcacactgcaagtgctggtcgatgccccagggaacatttcctgtctctgggtctttaagcacagctccctgaattgccagccacattttgatttacaaaacagaggagttgtttccatggtcattttgaaaatgacagaaacccaagctggagaatacctactttttattcagagtgaagctaccaattacacaatattgtttacagtgagtataagaaataccctgctttacacattaagaagaccttactttagaaaaatggaaaaccaggacgccctggtctgcatatctgagagcgttccagagccgatcgtggaatgggtgctttgcgattcacagggggaaagctgtaaagaagaaagtccagctgttgttaaaaaggaggaaaaagtgcttcatgaattatttgggatggacataaggtgctgtgccagaaatgaactgggcagggaatgcaccaggctgttcacaatagatctaaatcaaactcctcagaccacattgccacaattatttcttaaagtaggggaacccttatggataaggtgcaaagctgttcatgtgaaccatggattcgggctcacctgggaattagaaaacaaagcactcgaggagggcaactactttgagatgagtacctattcaacaaacagaactatgatacggattctgtttgcttttgtatcatcagtggcaagaaacgacaccggatactacacttgttcctcttcaaagcatcccagtcaatcagctttggttaccatcgtagaaaagggatttataaatgctaccaattcaagtgaagattatgaaattgaccaatatgaagagttttgtttttctgtcaggtttaaagcctacccacaaatcagatgtacgtggaccttctctcgaaaatcatttccttgtgagcaaaagggtcttgataacggatacagcatatccaagttttgcaatcataagcaccagccaggagaatatatattccatgcagaaaatgatgatgcccaatttaccaaaatgttcacgctgaatataagaaggaaacctcaagtgctcgcagaagcatcggcaagtcaggcgtcctgtttctcggatggatacccattaccatcttggacctggaagaagtgttcagacaagtctcccaactgcacagaagagatcacagaaggagtctggaatagaaaggctaacagaaaagtgtttggacagtgggtgtcgagcagtactctaaacatgagtgaagccataaaagggttcctggtcaagtgctgtgcatacaattcccttggcacatcttgtgagacgatccttttaaactctccaggccccttccctttcatccaagacaacatctcattctatgcaacaattggtgtttgtctcctcttcattgtcgttttaaccctgctaatttgtcacaagtacaaaaagcaatttaggtatgaaagccagctacagatggtacaggtgaccggctcctcagataatgagtacttctacgttgatttcagagaatatgaatatgatctcaaatgggagtttccaagagaaaatttagagtttgggaaggtactaggatcaggtgcttttggaaaagtgatgaacgcaacagcttatggaattagcaaaacaggagtctcaatccaggttgccgtcaaaatgctgaaagaaaaagcagacagctctgaaagagaggcactcatgtcagaactcaagatgatgacccagctgggaagccacgagaatattgtgaacctgctgggggcgtgcacactgtcaggaccaatttacttgatttttgaatactgttgctatggtgatcttctcaactatctaagaagtaaaagagaaaaatttcacaggacttggacagagattttcaaggaacacaatttcagtttttaccccactttccaatcacatccaaattccagcatgcctggttcaagagaagttcagatacacccggactcggatcaaatctcagggcttcatgggaattcatttcactctgaagatgaaattgaatatgaaaaccaaaaaaggctggaagaagaggaggacttgaatgtgcttacatttgaagatcttctttgctttgcatatcaagttgccaaaggaatggaatttctggaatttaagtcgtgtgttcacagagacctggccgccaggaacgtgcttgtcacccacgggaaagtggtgaagatatgtgactttggattggctcgagatatcatgagtgattccaactatgttgtcaggggcaatgcccgtctgcctgtaaaatggatggcccccgaaagcctgtttgaaggcatctacaccattaagagtgatgtctggtcatatggaatattactgtgggaaatcttctcacttggtgtgaatccttaccctggcattccggttgatgctaacttctacaaactgattcaaaatggatttaaaatggatcagccattttatgctacagaagaaatatacattataatgcaatcctgctgggcttttgactcaaggaaacggccatccttccctaatttgacttcgtttttaggatgtcagctggcagatgcagaagaagcgatgtatcagaatgtggatggccgtgtttcggaatgtcctcacacctaccaaaacaggcgacctttcagcagagagatggatttggggctactctctccgcaggctcaggtcgaagattcgtagaggaacaatttagttttaaggacttcatccctccacctatccctaacaggctgtagattaccaaaacaagattaatttcatcactaaaagaaaatctattatcaactgctgcttcaccagacttttctctagaagctgtctgcgtttactcttgttttcaaagggacttttgtaaaatcaaatcatcctgtcacaaggcaggaggagctgataatgaactttattggagcattgatctgcatccaaggccttctcaggctggcttgagtgaattgtgtacctgaagtacagtatattcttgtaaatacataaaacaaaagcattttgctaaggagaagctaatatgattttttaagtctatgttttaaaataatatgtaaatttttcagctatttagtgatatattttatgggtgggaataaaatttctaccgaggatcc

SEQ ID NO: 31 - DNA, 3848 bp, Homo sapiens
acctgcagcgcgaggcgcgccgctccaggcggcatcgcagggctgggccggcgcggcctggggaccccgggctccggaggccatgccggcgttggcgcgcgacggcggccagctgccgctgctcgttgttttttctgcaatgatatttgggactattacaaatcaagatctgcctgtgatcaagtgtgttttaatcaatcataagaacaatgattcatcagtggggaagtcatcatcatatcccatggtatcagaatccccggaagacctcgggtgtgcgttgagaccccagagctcagggacagtgtacgaagctgccgctgtggaagtggatgtatctgcttccatcacactgcaagtgctggtcgacgccccagggaacatttcctgtctctgggtctttaagcacagctccctgaattgccagccacattttgatttacaaaacagaggagttgtttccatggtcattttgaaaatgacagaaacccaagctggagaatacctactttttattcagagtgaagctaccaattacacaatattgtttacagtgagtataagaaataccctgctttacacattaagaagaccttactttagaaaaatggaaaaccaggacgccctggtctgcatatctgagagcgttccagagccgatcgtggaatgggtgctttgcgattcacagggggaaagctgtaaagaagaaagtccagctgttgttaaaaaggaggaaaaagtgcttcatgaattatttgggacggacataaggtgctgtgccagaaatgaactgggcagggaatgcaccaggctgttcacaatagatctaaatcaaactcctcagaccacattgccacaattatttcttaaagtaggggaacccttatggataaggtgcaaagctgttcatgtgaaccatggattcgggctcacctgggaattagaaaacaaagcactcgaggagggcaactactttgagatgagtacctattcaacaaacagaactatgatacggattctgtttgcttttgtatcatcagtggcaagaaacgacaccggatactacacttgttcctcttcaaagcatcccagtcaatcagctttggttaccatcgtagaaaagggatttataaatgctaccaattcaagtgaagattatgaaattgaccaatatgaagagttttgtttttctgtcaggtttaaagcctacccacaaatcagatgtacgtggaccttctctcgaaaatcatttccttgtgagcaaaagggtcttgataacggatacagcatatccaagttttgcaatcataagcaccagccaggagaatatatattccatgcagaaaatgatgatgcccaatttaccaaaatgttcacgctgaatataagaaggaaacctcaagtgctcgcagaagcatcggcaagtcaggcgtcctgtttctcggatggatacccattaccatcttggacctggaagaagtgttcagacaagtctcccaactgcacagaagagatcacagaaggagtctggaatagaaaggctaacagaaaagtgtttggacagtgggtgtcgagcagtactctaaacatgagtgaagccataaaagggttcctggtcaagtgctgtgcatacaattcccttggcacatcttgtgagacgatccttttaaactctccaggccccttccctttcatccaagacaacatctcattctatgcaacaattggtgtttgtctcctcttcattgtcgttttaaccctgctaatttgtcacaagtacaaaaagcaatttaggtatgaaagccagctacagatggtacaggtgaccggctcctcagataatgagtacttctacgttgatttcagagaatatgaatatgatctcaaatgggagtttccaagagaaaatttagagtttgggaaggtactaggatcaggtgcttttggaaaagtgatgaacgcaacagcttatggaattagcaaaacaggagtctcaatccaggttgccgtcaaaatgctgaaagaaaaagcagacagctctgaaagagaggcactcatgtcagaactcaagatgatgacccagctgggaagccacgagaatattgtgaacctgctgggggcgtgcacactgtcaggaccaatttacttgatttttgaatactgttgctatggtgatcttctcaactatctaagaagtaaaagagaaaaatttcacaggacttggacagagattttcaaggaacacaatttcagtttttaccccactttccaatcacatccaaattccagcatgcctggttcaagagaagttcagatacacccggactcggatcaaatctcagggcttcatgggaattcatttcactctgaagatgaaattgaatatgaaaaccaaaaaaggctggaagaagaggaggacttgaatgtgcttacatttgaagatcttctttgctttgcatatcaagttgccaaaggaatggaatttctggaatttaagtcgtgtgttcacagagacctggccgccaggaacgtgcttgtcacccacgggaaagtggtgaagatatgtgactttggattggctcgagatatcatgagtgattccaactatgttgtcaggggcaatgcccgtctgcctgtaaaatggatggcccccgaaagcctgtttgaaggcatctacaccattaagagtgatgtctggtcatatggaatattactgtgggaaatcttctcacttggtgtgaatccttaccctggcattccggttgatgctaacttctacaaactgattcaaaatggatttaaaatggatcagccattttatgctacagaagaaatatacattataatgcaatcctgctgggcttttgactcaaggaaacggccatccttccctaatttgacttcgtttttaggatgtcagctggcagatgcagaagaagcgatgtatcagaatgtggatggccgtgtttcggaatgtcctcacacctaccaaaacaggcgacctttcagcagagagatggatttggggctactctctccgcaggctcaggtcgaagattcgtagaggaacaatttagttttaaggacttcatccctccacctatccctaacaggctgtagattaccaaaacaagattaatttcatcactaaaagaaaatctattatcaactgctgcttcaccagacttttctctagaagctgtctgcgtttactcttgttttcaaagggacttttgtaaaatcaaatcatcctgtcacaaggcaggaggagctgataatgaactttattggagcattgatctgcatccaaggccttctcaggctggcttgagtgaattgtgtacctgaagtacagtatattcttgtaaatacataaaacaaaagcattttgctaaggagaagctaatatgattttttaagtctatgttttaaaataatatgtaaatttttcagctatttagtgatatattttatgggtgggaataaaatttctactacagaattgcccattattgaattatttacatggtataattagggcaagtcttaactggagttcacgaaccccctgaaattgtgcacccatagccacctacacattccttccagagcacgtgtgcttttaccccaagatacaaggaatgtgtaggcagctatggttgtcacagcctaagatttctgcaacaacaggggttgtattgggggaagtttataatgaataggtgttctaccataaagagtaatacatcacctagacactttggcggccttcccagactcagggccagtcagaagtaacatggaggattagtattttcaataaagttactcttgtccccacaaaaaaa

SEQ ID NO: 32 - protein, 993 aa, Homo sapiens
MPALARDGGQLPLLVVFSAMIFGTITNQDLPVIKCVLINHKNNDSSVGKSSSYPMVSESPEDLGCALRPQSSGTVYEAAAVEVDVSASITLQVLVDAPGNISCLWVFKHSSLNCQPHFDLQNRGVVSMVILKMTETQAGEYLLFIQSEATNYTILFTVSIRNTLLYTLRRPYFRKMENQDALVCISESVPEPIVEWVLCDSQGESCKEESPAVVKKEEKVLHELFGTDIRCCARNELGRECTRLFTIDLNQTPQTTLPQLFLKVGEPLWIRCKAVHVNHGFGLTWELENKALEEGNYFEMSTYSTNRTMIRILFAFVSSVARNDTGYYTCSSSKHPSQSALVTIVEKGFINATNSSEDYEIDQYEEFCFSVRFKAYPQIRCTWTFSRKSFPCEQKGLDNGYSISKFCNHKHQPGEYIFHAENDDAQFTKMFTLNIRRKPQVLAEASASQASCFSDGYPLPSWTWKKCSDKSPNCTEEITEGVWNRKANRKVFGQWVSSSTLNMSEAIKGFLVKCCAYNSLGTSCETILLNSPGPFPFIQDNISFYATIGVCLLFIVVLTLLICHKYKKQFRYESQLQMVQVTGSSDNEYFYVDFREYEYDLKWEFPRENLEFGKVLGSGAFGKVMNATAYGISKTGVSIQVAVKMLKEKADSSEREALMSELKMMTQLGSHENIVNLLGACTLSGPIYLIFEYCCYGDLLNYLRSKREKFHRTWTEIFKEHNFSFYPTFQSHPNSSMPGSREVQIHPDSDQISGLHGNSFHSEDEIEYENQKRLEEEEDLNVLTFEDLLCFAYQVAKGMEFLEFKSCVHRDLAARNVLVTHGKVVKICDFGLARDIMSDSNYVVRGNARLPVKWMAPESLFEGIYTIKSDVWSYGILLWEIFSLGVNPYPGIPVDANFYKLIQNGFKMDQPFYATEEIYIIMQSCWAFDSRKRPSFPNLTSFLGCQLADAEEAMYQNVDGRVSECPHTYQNRRPFSREMDLGLLSPQAQVEDS

SEQ ID NO: 33 - protein, 6 aa, Mus musculus
KAKFYN

SEQ ID NO: 34 - protein, 6 aa, Homo sapiens
HTHKTR

SEQ ID NO: 35 - DNA, 23 bp, Artificial sequence
  Synthetic sequence Primer
gcaatttaggtatgaaagccagc

SEQ ID NO: 36 - DNA, 23 bp, Artificial sequence
  Synthetic sequence Primer
ctttcagcattttgacggcaacc

SEQ ID NO: 37 - DNA, 20 bp, Artificial sequence
  Synthetic sequence Primer
gggacttctcaggtcgtgtc

SEQ ID NO: 38 - DNA, 20 bp, Artificial sequence
  Synthetic sequence Primer
agccccgtacttcttcaggt

SEQ ID NO: 39 - DNA, 20 bp, Artificial sequence
  Synthetic sequence Primer
gtggggcgccccaggcacca

SEQ ID NO: 40 - DNA, 24 bp, Artificial sequence
  Synthetic sequence Primer
ctccttaatgtcacgcacgatttc

SEQ ID NO: 41 - DNA, 25 bp, Artificial sequence
  Synthetic sequence Probe
ggtgatgttttctggaagtgtgggt

SEQ ID NO: 42 - DNA, 26 bp, Artificial sequence
  Synthetic sequence Probe
ccataagttcttggaagctgcggctt

SEQ ID NO: 43 - DNA, 21 bp, Artificial sequence
  Synthetic sequence STAT5 competitor sequence
agatttctaggaattcaatcc

(19) United States
(12) Patent Application Publication
(10) Pub. No.: US 2017/0146538 A1
(43) Pub. Date: May 25, 2017

(54) PRL-3 AS A BIOMARKER FOR THE PROGNOSIS OF CANCER AND A TARGET FOR THERAPY

(71) Applicant: Agency for Science, Technology and Research, Singapore (SG)

(72) Inventor: Qi Zeng, Singapore (SG)

(73) Assignees: Agency for Science, Technology and Research, Singapore (SG); Agency for Science, Technology and Research, Singapore (SG)

(21) Appl. No.: 15/116,956

(22) PCT Filed: Feb. 7, 2014
(86) PCT No.: PCT/SG14/00052

§ 371 Date: Aug. 5, 2016

(51) Int. Cl. G01N 33/574; C07K 16/30

(52) U.S. Cl. CPC G01N 33/57426; C07K 16/3061; G01N 2333/916; A61K 2039/505

(57) ABSTRACT

Previously, we have shown that a cancer associated-PRL-3 intracellular phosphatase is a potential therapeutic target for PRL-3 antibody therapy. PRL-3 has recently emerged as a potentially useful biomarker for cancer prognosis, particularly the prediction of cancer metastasis (Matsukawa et al, 2010, Ren et al, 12 2009). Here we demonstrate that PRL-3 can act as an independent prognostic marker for cancers.

### FIELD OF THE INVENTION

The present invention relates to PRL3 and particularly, although not exclusively, to anti-PRL3 antibodies, and methods for the prognosis of cancer through the detection and quantification of PRL3.

### BACKGROUND TO THE INVENTION

Acute myeloid leukemia (AML) is characterized by a block in differentiation and uncontrolled proliferation of malignant clones of immature myelold cells (Lowenberg et al, 1999). Because of the high heterogeneity of acquired mutations occurring through unknown mechanisms, therapeutic approaches have limited efficacies and clinical outcomes of AML patients are poor (Steinberg & Licht, 2005). Activating mutations in fms-like tyrosine kinase-3 (FLT3) represent one of the more frequent genetic alterations in AML (Rockova at al, 2011), involving internal tandem duplication (ITD) in the juxtamembrane (JM) domain of FLT3 (Nakao et al, 1996). The constitutive activation of FLT3-ITD leads to elevated and sustained activation of multiple downstream signalling pathways, ultimately resulting in the transformation of hematopoietic cells to growth factor-independent proliferation (Mizuki et al. 2000). Due to their essential pro-proliferative and anti-apoptotic roles in AML cells, activating mutations in FLT3 have been proposed as a promising molecular target for the treatment of AML. However, despite advances in drug discovery and our understanding of the molecular mechanism of FLT3 mutations, clinical trials with FLT3 inhibitors so far have shown limited success due to drug resistance and poor clinical response (Weisberg et at, 2010). This suggests that understanding of the underlying mechanism of FLT3 mutations may help in the development of better therapeutic strategies.

The FLT3-ITD mutations are detected in 25-30% of AML patients and are associated with poor prognosis. Targeting FLT3-ITD mutations are a promising therapeutic approach for AML, however, clinical trials with FLT3 inhibitors have showed limited success. Insights into how FLT3 mutation leads to the disease will offer novel therapeutic opportunities.

Previously, we have shown that a cancer associated-PRL-3 intracellular phosphatase is a potential therapeutic target for PRL-3 antibody therapy. PRL-3 is one of the three members (PRL-1, -2, and -3) In the PRL (phosphatase of regenerating liver) family which was identified in 1994 and 1998. The three PRLs form a subgroup of the protein tyrosine phosphatase (PTP) family.

Previously, PRL-3 was first discovered to be specifically up-regulated in metastatic colorectal cancer cells (Saha et al., 2001) and subsequently reported to be associated with many other types of cancer metastasis such as breast liver and gastric cancers (Bessette et al, 2008). Diverse roles of PRL-3 in cancer progression, including cell migration, invasion, proliferation, angiogenesis, and metastasis, have been highlighted in recent reports that emphasize the importance of PRL-3 in tumorigenesis (Al-Aidaroos & Zeng, 2010; Liang et al, 10 2007).

PRL-3 has recently emerged as a potentially useful biomarker for cancer prognosis, particularly the prediction of cancer metastasis (Matsukawa et al, 2010; Ren et al, 12 2009). Herein we demonstrate that PRL-3 can act as an independent prognostic marker for cancers.

### SUMMARY OF THE INVENTION

The present invention provides a method for the prognosis of cancer in an individual. The method may involve determining the survival rate of an individual. The method may involve determining the expression, activity, or level or PRL3 in a sample from the patient. The method may involve determining whether the expression, activity or level of PRL3 in the sample is modulated. Modulation may be determined relative to a control, such as a non-cancerous sample from the individual, or from another individual that does not have cancer.

We provide a method for the prognosis of cancer, that is to say, a method for predicting the outcome of a patient's cancer. The method comprises determining the amount of PRL3 protein or PRL3 nucleic acid in a sample obtained from the patient and comparing the amount of PRL3 protein or PRL3 nucleic acid to the amount of PRL3 protein or PRL3 nucleic in a control. A higher level of PRL3 in the sample obtained from the patient than in the control is indicative of a poor prognosis. In some cases, a level of 10% more PRL3 in the sample obtained from the patient than in the control is indicative of a poor prognosis.

In some cases, the method is used for the prognosis of the outcome of a patient with leukemia. In some cases, the leukemia is myeloid leukemia, such as acute myeloid leukemia or chronic myeloid leukemia.

The method may be carried out on a sample of bodily fluid that has been obtained from the patient. The sample may be a bone marrow sample, such as a bone marrow aspirate. In some cases, the prognosis is made for a patient that has a normal karyotype, or has a cancer that has a normal karyotype. The method of prognosis may involve the step of determining the karyotype of the patient.

The prognosis may be performed for a patient that has a FLT3-ITD positive cancer. The method may involve the step of determining that the sample is a FLT3-ITD positive sample. The cancer may have been previously determined to have a FLT3-ITD positive cancer. The patient may have been previously treated with, or may be undergoing, FLT3 inhibition therapy. In some cases, the FLT3 inhibition therapy may not have been successful, or may have been only partially successful.

In the prognosis method described herein, PRL3 protein levels in the sample may be determined by immunoassay. In some cases, PRL3 nucleic acid levels, particularly PRL3 mRNA levels are determined by qRT-PCR.

Also described herein are anti-PRL3 antibodies for use in a method of treatment of leukemia, and the use of anti-PRL3 antibodies for the manufacture of a medicament for the treatment of leukemia. The leukemia may be myeloid leukemia, such as acute myeloid leukemia or chronic myeloid leukemia.

In some cases, the anti-PRL3 antibody may be provided for use in the treatment of a patient that has previously undergone, or is undergoing, FLT3 inhibition therapy, such as Linifanib or Linifanib and SAHA therapy. The patient may not have responded to that therapy, or may have only partially responded to that therapy.

The anti-PRL3 antibodies described herein, the methods of treatment involving the administration of anti-PRL3 antibodies may additionally involve the administration of a compound that inhibits FLT3, and particularly which inhibits FLT3-ITD, such as Linifanib.

### DESCRIPTION

The invention includes the combination of the aspects and preferred features described except where such a combination is clearly impermissible or expressly avoided.

The section headings used herein are for organizational purposes only and are not to be construed as limiting the subject matter described.

All documents mentioned in this text are incorporated herein by reference.

As disclosed herein, we investigated the role of PRL-3 in FLT3-ITD positive AML cells and patient samples. We describe the regulation of PRL-3 by a FLT3-Src-STAT5 signalling in AML cells. PRL-3 expression correlated positively with FLT3-ITD mutation in AML patients. PRL-3 overexpression was associated with the activation of c-Jun proto-oncogene and cell growth. Finally, we describe the clinical relationship between elevated PRL-3 expression and shorter overall survival in AML patients, and characterize elevated PRL-3 expression as an independent prognostic marker for AML. A critical role of PRL-3 in leukemogenesis was revealed using PRL-3-targeted immunotherapy in a leukemic mouse model, suggesting that PRL-3 could be a potential therapeutic target for AML.

This study investigated the regulation and function of PRL-3, a metastasis-associated phosphatase, in leukemia cell lines and AML patient samples associated with FLT3-ITD mutations. PRL-3 overexpression is mediated by the FLT3-Src-STAT5 signalling pathway in leukemia cells, results in an activation of the AP-1 transcription factors via the ERK and JNK pathway. Depletion of PRL-3 attenuates cell growth and cell cycle progression in vitro whereas overexpression of PRL-3 enhances leukemia development in vivo. PRL-3 antibody therapy reduced tumor burden in a leukemia mouse model. The FLT3-ITD mutation was clinically associated with an increase in PRL-3 expression in four independent cohorts in a total of 1158 AML patients. Higher PRL-3 expression was significantly (p≦0.001) associated with shorter survival in AML patients.

The mechanistic findings on the FLT3-ITD-STAT5 signalling-mediated PRL-3 regulation unveiled the underlying mechanism of elevated PRL-3 expression that results in cell growth and tumor burden. Targeting PRL-3 reversed the oncogenic effects in FLT3-ITD AML models in vitro and in vivo, suggesting that PRL-3 is a promising therapeutic target. Performing multivariable Cox-regression in 221 AML patients of Cohort 1 identified PRL-3 as a novel prognostic marker independent of other clinical parameters.

### BRIEF DESCRIPTION OF THE FIGURES

FIG. 1. PRL-3 mRNA levels are elevated in FLT3-ITD-positive AML samples A. RT-PCR analysis of PRL-3 mRNA expression levels in 19 bone marrow samples from AML patients either negative (ITD NEG; n=12) or positive (ITD POS; n=7) for FLT3-ITD mutation. MOLM-14 and MV4-11 AML cell lines were used as FLT3-ITD positive controls. β-actin, loading control. B. (a-d) Microarray data analysis of PRL-3 mRNA levels in FLT-ITD-positive (POS) or FLT3-ITD-negative (NEG) patients in four independent patient cohorts (total n=1158). a. Cohort 1 AML patient with normal karyotype (n=101, p=0.001). b. GSE1159 AML patient cohort (n=285, p<0.001). c. GSE6891 AML patient cohort (n=521, p<0.001). d. GSE15434 AML patient cohort (n=251, p<0.001). Statistical differences between ITD-POS and ITD-NEG patients were determined using Chi-square test. PRL-3 expression level is divided into 4 groups; Very high, High, Intermediate, Low C. Western blot analysis of PRL-3 protein levels in four AML cell lines. D. Western blot analysis of PRL-3 in MOLM-14 and MV4-11 cells upon siRNA-mediated knock-down of FLT3 expression. NS, control non-silencing siRNA. GAPDH, loading control.

FIG. 2. PRL-3 protein expression decreases upon FLT3 or Src inhibition in AML cell lines TF1-ITD and MOLM-14 cells were incubated with various concentrations of FLT3 inhibitors (PKC412, CEP-701) or Src inhibitors (SU6656, PP2) for 24 h. Whole cell lysates were subjected to western blot analysis with indicated antibodies. GAPDH, loading control. A. (a-d) Western blot analysis of AML cells upon FLT3 inhibition. PKC412 and CEP-701 inhibited the phosphorylation of both FLT3 and STAT5 as well as PRL-3 protein levels in a dose-dependent manner in both TF1-ITD (a-b) and MOLM-14 (c-d) cells B. (a-d) Western blot analysis of AML cells upon FLT3 inhibition. PKC412 and CEP-701 inhibited the phosphorylation of Src, but not JAK, in a dose-dependent manner in both TF1-ITD (a-b) and MOLM-14 (c-d) cells. C. (a-b) Western blot analysis of AML cells upon Src inhibition. SU6656 (a) and PP2 (b) inhibited the phosphorylation of Src and STAT5 as well as PRL-3 protein levels in a dose-dependent manner in TF1-ITD cells.

FIG. 3. STAT5A is a direct transcriptional regulator of PRL-3 expression. A. Two putative STAT5 binding sites (S1 and S2; DNA sequences illustrated) in a distal 5′-flanking region of PRL-3, as predicted by TRANSFAC. B. EMSA analysis using S1 and $2 biotinylated DNA probes (S1 and S2) incubated with nuclear extracts from either TF-1 or TF1-ITD cells. Arrow, shifted protein/probe complex. C. EMSA analysis as in (B) in the presence of 10-fold molar excess of unlabelled STAT5 competitor. D. Western blot analysis of streptavidin-agarose pull-down fractions (unbound or bound) using probe S1. E. Left panel, schematic diagram of a −5.4 kb upstream sequence of PRL-3 and its 5′-sequential deletion sequence with luciferase reporter vector (pGL3-S1a, S1b, S1c, and S1d), respectively. Right panel, STAT5A or STAT5B expression vectors were co-transfected with PRL-3 luciferase reporter vector to TF-1 cells and luciferase activity measured. Error bars represent the mean±SD from three independent experiments. F. PRL-3 expression is downregulated upon siRNA-mediated STAT5 depletion in AML cells. NS, control non-silencing siRNA. (a) Quantitative real time PCR analysis of PRL-3 mRNA level after knock-down of STAT5 gene, normalized to GAPDH mRNA. Statistical differences between two groups were determined using Student's t-test (mean±SD, n=3, **p<0.01). (b) Western blot analysis of PRL-3 protein level after knock-down of STAT5 gene. GAPDH, loading control.

FIG. 4. PRL-3 specifically activates c-Jun through ERK and JNK signaling pathways. A. SEAP reporter assay results measuring AP-1 activity in TF-1 cells overexpressing GFP (TF1-GFP) or GFP-PRL-3 (TF1-PRL-3) (mean±SD, n=3). B. (a-b) PRL-3 specifically upregulates c-Jun but not c-Fos. (a) Western blot analysis of TF1-GFP, TF1-PRL-3 and TF1-ITD cells. (b) Western blot analysis after knock-down of endogenous PRL-3 in TF1-ITD cells. GAPDH, loading control. NS, control non-silencing siRNA. C. (a-d) PRL-3-mediated upregulation of c-Jun is dependent on ERK and JNK pathways. (a) Western blot analysis after knock-down of endogenous PRL-3 in TF1-ITD and MOLM-14 cells. (b) Western blot analysis of TF1-GFP and TF1-PRL-3 cells. (c-d) Western blot analysis after knock-down of ERK1/2 or JNK in TF1-PRL-3 cells. GAPDH, loading control. NS, control non-silencing siRNA. D. (a-c) MTS assay results reflecting numbers of viable TF1-PRL-3 cells after treatment with ERK-specific inhibitor (U0126), JNK-specific inhibitor (SP600125), or general AP-1 inhibitor (curcumin) for the various time points. Error bars represent the mean±SD from three independent experiments.

FIG. 5. PRL-3 promotes growth and suppresses apoptosis of TF-1 leukemia cells upon cytokine deprivation. A. Right panel, MTS assay results reflecting numbers of viable TF1-GFP and TF1-PRL-3 cells after culture in the absence of cytokines for various durations. Error bars represent the mean±SD from three independent experiments. Left panel, western blot analysis of TF1-GFP and TF1-PRL-3 cells. GAPDH, loading control. B. Flow cytometry analysis of propidium iodide-stained TF1-GFP and TF1-PRL-3 after 48 h culture in the absence of cytokines. Note the difference in sub-G1 peak/population, reflective of apoptotic cells. Representative data from three independent 0.5 experiments are shown. C. Left panel, flow cytometry analysis of annexin-V- and 7-AAD-stained TF1-GFP and TF1-PRL-3 after 48 h culture in the absence of cytokines. The percentage in the upper left quadrant indicates the fraction of annexin-V-positive apoptotic cells in the entire cell population analyzed. Right panel, quantitation of annexin-V-positive apoptotic population in three independent experiments. Statistical differences between two groups were determined using Student's t-test (mean±SD, n=3, p<0.001).

FIG. 6. PRL-3 depletion inhibits the growth of FLT3-ITD-positive AML cells The growth of PRL-3-depleted MOLM-14 and MV4-11 FLT3-ITD-positive AML cells was analyzed by MTS assay and flow cytometry. A. a. Knock-down of PRL-3 decreased cell number in FLT3-ITD positive MOLM-14 cells (mean±SD; n=3). b. Depletion of PRL-3 accumulated cells in G1 phase in MOLM-14 cells. B. a. Knock-down of PRL-3 decreased cell number in FLT3-ITD positive MV4-11 cells (mean±SD, n=3). b. Depletion of PRL-3 accumulated cells in G1 phase in MV4-11 cells. Representative data (right panel) from three independent experiments are shown.

FIG. 7. PRL-3 mAb exerts anti-tumor therapeutic effects in a mouse model of AML A. (a-b) Results of immunotherapy on liver and spleen sizes in a mouse model of AML. (a) Representative images of livers and spleens harvested from normal nude mice (upper left panel) or nude mice 12-14 days after i.v. injection of TF1-ITD cells together with bi-weekly i.v. administration of control IgG (upper right panel), PRL-3 mAb (lower left panel) or FLT3 mAb (lower right panel). (b) Quantitation of liver and spleen weights of mice as described in (a). Statistical differences between data groups were determined using Student's t-test (mean±SD, n=11. **p<0.001). B. (a-b) Results of immunotherapy and PRL-3 knock-down on leukemic infiltration in mouse bone marrow (BM) cells in a mouse model of AML. (a) BM cells from nude mice 12-14 days after i.v. injection of (I) TF1, TF1-ITD cells together with bi-weekly i.v. administration of (11) control IgG or (Ill) PRL-3 mAb (lower left panel), or (IV) TF1-ITD cells depleted of endogenous SPRL-3 were analyzed using flow cytometry analysis using the human-specific marker CD45+ to distinguish TF1 human-derived AML cells. Percentages indicate proportion of CD45+ cells in the BM population analyzed. (b) Quantitation of CD45+ engrafted cells as described in (a). Statistical differences between two groups were determined using Student's t-test (mean±SD, n=5, **p<0.0001). C. Kaplan-Meier survival analysis of PRL-3 mAb-treated (n=7) or control IgG-treated (n=7) mice in the TF1-ITD leukemia mouse model (p<0.001).

FIG. 8. Elevated expression of PRL-3 correlates with a shorter survival in three independent AML patient cohorts. Kaplan-Meier analysis of overall survival (OS) in normal karyotype AML patients for PRL-3 mRNA expression in (A) Cohort 1 AML patients, n=101, (B) GSE6891, n=227, and (C) GSE12417, n=163. Statistically significant p-values (using the log-rank test) are indicated in the figures.

FIG. 9. Multivariate cox-regression analysis reveals PRL-3 as an independent prognostic marker. PRL-3 acts a novel prognostic marker in AML. By Multivariate Cox-regression analysis using backward conditional stepwise method with a removal, limit of p>0.05. PRL-3 constituted one of the key independent predictors for poorer patient survival in our Cohort 1 (n=221).

FIG. 10. Details of all human AML patients detasets used in this study.

PRL-3, a phosphatase that we identified in 1998 (Zeng et al, 1998), was recently found as part of a core gene signature that is uniquely down-regulated by combination therapy of Linifanib (ABT-869, a FLT3 inhibitor) and suberoylanilide hydroxanic acid (SAHA, a histone deacetylase inhibitor) in AML cells (Zhou et al, 2011). Intriguingly, PRL-3 was recently reported as a possible downstream target of FLT3-ITD signalling, with a potential role in drug resistance of leukemia cells (Zhou et at, 2011), indicating that PRL-3 expression levels could be an important factor contributing to the outcomes of the AML treatments.

FIG. 11. High PRL-3 mRNA expression was associated with AML patients with FLT3-ITD mutation. PRL-3 mRNA levels were assessed in 19 AML patients' bone marrow samples by quantitative real-time PCR (qRT-PCR) analysis. Up-regulation of PRL-3 mRNA was shown in patient #1, #6, and #10 with FLT3-ITD negative mutation (NEG, n=12), and in patient #13, #15, #17, #18, and #19 with FLT3-LTD positive mutation (POS, n=7). For quantification of relative PRL-3 mRNA level, patient #1 was set as 1 for reference. Error bars represent the mean±SD from three independent experiments. NEG, negative; POS, positive.

FIG. 12. Similar STAT5A and STAT5B protein expression levels were detected in TF-1 cells expressing different reporter constructs.

For the luciferase reporter assay, pCMV6-STAT5A or pCMV6-STAT5B expression vector was co-transfected respectively with pGL-Luc-S1a, -S1b, -S1c, -or -S1d constructs in TF-1 cells. Western blots showed similar expression levels of STAT5A and STAT5B at all conditions.

FIG. 13. PRL-3 overexpression activates AP-1 activity.

Activation of AP-1 activity was examined using two solid tumor cell lines. DLD-1 and HCT116. Each cell line was transiently co-transfected with AP-1 SEAP reporter vector along with either GFP or GFP-PRL-3 expression vector. Overexpression of PRL-3 led to a 6.5-fold and >2.5-fold increase in AP-1 activity in DLD-1 and HCT116 cells, respectively. Error bars represent the mean±SD from three independent experiments.

FIG. 14. Depletion of PRL-3 shows no substantial increment in apoptotic population in two cytokine independent cell lines, MOLM-14 and MV4.11.

Apoptotic activity of PRL-3 was assessed by Annexin-V and 7-AAD staining, followed by FACS analysis. The populations of Annexin V-positive cells are shown on top left corner of each panel. MOLM-14 and MV4-11 mock-knock down cells showed around 7.9% and 10.4% of Annexin-V positive cells, and PRL-3 depleted MOLM-14 and MV4-11 cells (MOLM-14 PRL-3 KD and MV4-11 PRL-3 KD) showed ˜10.3% and -12.6% of apoptotic population. A Left panel, flow cytometry analysis of annexin-V- and 7-AAD-stained MOLM-14 and MOLM-14 PRL-3-KD cells after 48 hr culture. Right panel, quantitation of annexin-V-positive apoptotic population in three independent experiments (mean±SD, n=3). B. Left panel, flow cytometry analysis of annexin-V- and 7-AAD-stained MV4-11 and MV4-11 PRL-3-KD cells after 48 hr culture, Right panel, quantitation of annexin-V-positive apoptotic population in three independent experiments (mean±SD, n=3).

FIG. 15. Sequence of human PRL3

FIG. 16. Sequences of antibody variable domains

The details of one, or more embodiments of the invention are set forth in the accompanying description below including specific details of the best mode contemplated by the inventors for carrying out the invention, by way of example. It will be apparent to one skilled in the art that the present invention may be practiced without limitation to these specific details.

PRL-3

PRL-3 is also known as Protein-tyrosine Phosphatase Type 4A, 3; PTP4A3. The chromosomal location of PRL-3 is at gene map locus 8q24.3. PRL-3 is one of the three members (PRL-1, -2, and -3) in the PRL (phosphatase of regenerating liver) family which was identified in 1994 and 1998^{5,6}. The three PRLs form a subgroup of the protein tyrosine phosphatase (PTP) family⁷.

In the heart, protein kinases regulate contractility, ion transport, metabolism and gene expression. Phosphatases, in addition to their role in dephosphorylation, are involved in cardiac hypertrophy and dysfunction.

PRL-3 was first linked to colorectal cancer metastasis in 2001. Saha et al (2001. Science 294: 1343-1346) compared global gene expression profiles of metastatic colorectal cancer with that of primary cancers, benign colorectal tumours and colorectal epithelium. PRL3 was expressed at high levels in each of 18 cancer metastases studied but at lower levels in nonmetastatic tumors and normal colorectal epithelium. In 3 of 12 metastases examined multiple copies of PRL3 gene were found within a small amplicon located at chromosome 8q24.3. Saha et al concluded that the PRL3 gene is important for colorectal cancer metastasis. Subsequently, up-regulation of individual PRLs-PTPs was reported to be correlated with numerous types of advanced human metastatic cancers when compared with their normal counterparts⁹.

The PRL phosphatases represent an intriguing group of proteins being validated as biomarkers and therapeutic targets in human cancers¹⁰. PRLs are intracellular C-terminally prenylated phosphatases, while mutant forms of PRLs that lack the prenylation signal are often localized in nuclei^{11,12}.

The localization of PRL-1 and PRL-3 to the inner leaflet of the plasma membrane and early endosomes has been revealed by EM immunogold labeling¹³. Targeting PRLs by exogenous reagents to ablate PRLs-cancer cells requires their penetration into cells and is a challenging task.

The methods and compositions described here make use of PRL-3 polypeptides, which are described in detail below. As used here, the term “PRL-3” is intended to refer to a sequence selected from the following.

| Unigene | Version | Description |
|---|---|---|
| AF041434.1 | GI:3406429 | Homo sapiens potentially prenylated protein tyrosine |
| | | phosphatase hPRL-3 mRNA, complete cds |
| BT007303.1 | GI:30583444 | Homo sapiens protein tyrosine phosphatase type IVA, |
| | | member 3 mRNA, complete cds |
| AK128380.1 | GI:34535719 | Homo sapiens cDNA FLJ46523 fis, clone |
| | | THYMU3034099 |
| NM_007079.2 | GI:14589853 | Homo sapiens protein tyrosine phosphatase type IVA, |
| | | member 3 (PTP4A3), transcript variant 2, mRNA |
| AY819648.1 | GI:55977462 | Homo sapiens HCV p7-transregulated protein 2 mRNA, |
| | | complete cds |
| BC003105.1 | GI:13111874 | Homo sapiens protein tyrosine phosphatase type IVA, |
| | | member 3, mRNA (cDNA clone MGC:1950 |
| | | IMAGE:3357244), complete cds |
| NM_032611.1 | GI:14589855 | Homo sapiens protein tyrosine phosphatase type IVA, |
| | | member 3 (PTP4A3), transcript variant 1, mRNA |
| AK311257.1 | GI:164696021 | Homo sapiens cDNA, FLJ 18299 |
| U87168.1 | GI:1842085 | Human protein tyrosine phosphatase homolog hPRL-R |
| | | mRNA, partial cds |
| BC066043.1 | GI:42406367 | Mus musculus protein tyrosine phosphatase 4a3, mRNA |
| | | (cDNA clone MGC:90066 IMAGE:6415021), |
| | | complete cds |
| AJ276554.1 | GI:26985935 | Homo sapiens mRNA for protein tyrosine phosphatase |
| | | hPRL-3, short form |
| AK190358.1 | GI:56014535 | Mus musculus cDNA, clone:YIG0102103, strand:plus, |
| | | reference:ENSEMBL:Mouse-Transcript- |
| | | ENST:ENSMUST00000053232, based on BLAT search |
| CT010215.1 | GI:71059758 | Mus musculus full open reading frame cDNA clone |
| | | RZPDo836H0950D for gene Ptp4a3, Protein tyrosine |
| | | phosphatase 4a3; complete cds, incl. stopcodon |
| AK147489.1 | GI:74184679 | Mus musculus adult male brain UNDEFINED CELL |
| | | LINE cDNA, RIKEN full-length enriched library, |
| | | clone:M5C1053F14 product:protein tyrosine phosphatase |
| | | 4a3, full insert sequence |
| AK172192.1 | GI:74182510 | Mus musculus activated spleen cDNA. RIKEN full-length |
| | | enriched library, clone:F830102P03 product:protein |
| | | tyrosine phosphatase 4a3, full insert sequence |
| AK 143702.1 | GI:74160753 | Mus musculus 6 days neonate spleen cDNA, RIKEN |
| | | full-length enriched library, clone:F43001 1 C20 |
| | | productprotein tyrosine phosphatase 4a3, full insert |
| | | sequence |
| AF035645.1 | GI:2992631 | Mus musculus potentially prenylated protein tyrosine |
| | | phosphatase mPRL-3 (Prl3) mRNA, complete cds |
| NM_008975.2 | GI:31543526 | Mus musculus protein tyrosine phosphatase 4a3 (Ptp4a3), |
| | | mRNA |
| AK014601.1 | GI:12852557 | Mus musculus 0 day neonate skin cDNA, RIKEN |
| | | full-length enriched library, clone:4632430E19 |
| | | product:protein tyrosine phosphatase 4a3, full insert |
| | | sequence |
| AK004562.1 | GI:12835815 | Mus musculus adult male lung cDNA, R1KEN full-length |
| | | enriched library, clone:1200003F10 productprotein |
| | | tyrosine phosphatase 4a3, full insert sequence |
| AK003954.1 | GI:12834926 | Mus musculus 18-day embryo whole body cDNA, RIKEN |
| | | full-length enriched library, clone:1110029E17 |
| | | productprotein tyrosine phosphatase 4a3, full insert |
| | | sequence |
| BC027445.1 | GI:20071662 | Mus musculus protein tyrosine phosphatase 4a3, mRNA |
| | | (cDNA clone MGC:36146 IMAGE:4482106), |
| | | complete cds |

A “PRL-3 polypeptide” may comprise or consist of a human PRL-3 polypeptide, such as the sequence having Unigene accession number AF041434.1.

Homologues variants and derivatives thereof of any, some or all of these polypeptides are also included. For example, PRL-3 may include Unigene Accession Number BC066043.1.

Variants, Derivatives and Homologues of PRL3 Polypeptides

The methods described herein may involve the detection or quantification of PRL3 polypeptides, or variants, homologues or derivatives of such polypeptides.

Cellular PRL3 may not be identical to a known PRL3 sequence as set out herein, and may carry or more mutations relative to a known sequence.

Thus, such sequences are not limited to the particular sequences set forth in this document, but also include homologous sequences, for example related cellular homologues, homologues from other species and variants or derivatives thereof.

This disclosure therefore encompasses variants, homologues or derivatives of the amino acid sequences set forth in this document, as well as variants, homologues or derivatives of the amino acid sequences encoded by the nucleotide sequences disclosed here.

The terms “variant” or “derivative” in relation to the amino acid sequences as described here includes any substitution of, variation of, modification of, replacement of, deletion of or addition of one (or more) amino acids from or to the sequence. The resultant amino acid sequence may retain substantially the same activity as the unmodified sequence, or the specific sequence variant may be associated with the cancerous phenotype of the cell.

Natural variants of intracellular PRL3 may comprise conservative amino acid substitutions. Conservative substitutions may be defined, for example according to the Table below. Amino acids in the same block in the second column such as those in the same line in the third column may be substituted for each other:

| | | |
|---|---|---|
| ALIPHATIC | Non-Polar | GAP |
| | | ILV |
| | Polar-uncharged | CSTM |
| | | NQ |
| | Polar-charged | DE |
| | | KR |
| AROMATIC | | HFWY |

The methods do not necessarily require the detection or quantification of complete polypeptides, variants, homologues or derivatives, and may instead involve the detection or quantification of fragments.

As indicated above, with respect to sequence identity, a “homologue” has such as at least 5% identity, at least 10% identity, at least 15% identity, at least 20% identity, at least 25% identity, at least 30% identity, at least 35% identity, at least 40% identity, at least 45% identity, at least 50% identity, at least 55% identity, at least 60% identity, at least 65% identity, at least 70% identity, at least 75% identity, at least 80% identity, at least 85% identity, at least 90% identity, or at least 95% identity to a relevant sequence.

Polynucleotides.

The methods of the invention may involve the detection of PRL-3 polynucleotides. These may comprise DNA or RNA.

Where the polynucleotide is double-stranded, both strands of the duplex, either individually or in combination may be detected. Where the polynucleotide is single-stranded, it is to be understood that the complementary sequence of that polynucleotide is also included.

The methods may involve the detection or quantification of chromosomal DNA, cellular DNA, mRNA, tRNA, cDNA or other nucleic acid.

Variants, Homologues and Derivatives of PRL3 Polynucleotides

The terms “variant”, “homologue” or “derivative” in relation to a nucleotide sequence described in this document include any substitution of, variation of, modification of, replacement of, deletion of or addition of one (or more) nucleotides from or to the sequence. The polynucleotide may be truncated or extended relative to PRL3 in noncancerous tissue.

As indicated above, with respect to sequence identity, a “homologue” has such as at least 5% identity, at least 10% identity, at least 15% identity, at least 20% identity, at least 25% identity, at least 30% identity, at least 35% identity, at least 40% identity, at least 45% identity, at least 50% identity, at least 55% identity, at least 60% identity, at least 65% identity, at least 70% Identity, at least 75% identity, at least 80% identity, at least 85% identity, at least 90% identity, or at least 95% identity to a relevant sequence.

It is not necessary that complete or intact nucleic acid is detected. The methods may, instead, involve the detection or quantification of fragments of PRL3 polynucleotides.

Patient

The patient to be treated may be any animal or human. The patient is preferably a non-human mammal, more preferably a human patient. The patient may be male or female. The patient may have, or may be suspected of having a cancer.

Sample

Methods described herein may be performed on a sample that has been obtained from a patient. Such methods may thus be performed ex vivo. They may be performed in vitro.

A sample may be taken from any tissue or bodily fluid. The sample may be a sample of cancerous tissue, such as a tumor sample or biopsy. The sample may have been removed during a surgical procedure, such as a tumorectomy or lumpectomy.

In some preferred arrangements, the method is performed on a bone marrow sample or biopsy. A bone marrow sample preferably contains bone marrow blast cells. The bone marrow sample may be taken from the pelvic or breast bone, or any other suitable bone; The sample or biopsy may be obtained using a needle. The bone marrow biopsy may be a trephine biopsy. In some cases, a bone marrow aspirate sample is obtained. In a bone marrow aspiration liquid bone marrow is removed from an individual.

In some arrangements the sample is taken from a bodily fluid, more preferably one that circulates through the body. Accordingly, the sample may be a blood sample or lymph sample.

The sample may comprise or may be derived from: a quantity of blood; a quantity of serum derived from the individual's blood which may comprise the fluid portion of the blood obtained after removal of the fibrin clot and blood cells; a quantity of pancreatic juice; a tissue sample or biopsy; or cells isolated from said individual.

The sample may be a blood sample or blood-derived sample. The blood derived sample may be a selected fraction of a patient's blood, e.g. a selected cell-containing fraction or a plasma or serum fraction.

A selected cell-containing fraction may contain cell types of interest which may include white blood cells (WBC), particularly peripheral blood mononuclear cells (PBC) and/or granulocytes, and/or red blood cells (RBC). Accordingly, methods according to the present invention may involve detection of a PRL3 polypeptide or nucleic acid in the blood, in white blood cells, peripheral blood mononuclear cells, granulocytes and/or red blood cells.

Prognosis

Prognosis, prognosing and prognose refer to estimating the risk of future outcomes in an individual based on their clinical and non-clinical characteristics. In particular, a method of determining the prognosis as used herein refers to the prediction of the outcome of, or future course of, an individual's or patient's cancer. Prognosis includes the prediction of patient's survival. Prognosis may be useful for determining an appropriate therapeutic treatment. Prognostic testing may be undertaken with (e.g. at the same time as) the diagnosis of a previously undiagnosed cancerous condition, or may relate to an existing (previously diagnosed) condition.

The method of prognosis may be an in vitro method performed on the patient sample, or following processing of the patient sample. Once the sample is collected, the patient is not required to be present for the in vitro method of prognosis to be performed and therefore the method may be one which is not practised on the human or animal body.

As disclosed herein, the level of PRL3 expression may be used to indicate the prognosis of patient's cancer. As described herein, elevated PRL3 expression and activity correlated with a shorter overall survival. Thus, upregulation of PRL3 gene expression or increased level of PRL3 protein may indicate poor prognosis such as reduced survival time.

Thus, methods of prognosis described herein involve the identification of, or quantification of, the expression or activity of PRL3. The methods may involve the detection of PRL3 protein or DNA or RNA encoding PRL3. The methods may involve the detection of PRL3 mRNA. Alternatively, the methods may involve the detection of PRL3 protein. The methods may involve quantification of PRL3.

It will be appreciated that, as the level of PRL-3 varies with the aggressiveness of a tumour, detection of PRL-3 expression, amount or activity may also be used to predict a survival rate of an individual with cancer, i.e., high levels of PRL-3 indicating a lower survival rate or probability and low levels of PRL-3 indicating a higher survival rate or probability, both as compared to individuals or cognate populations with normal levels of PRL-3. Detection of expression, amount or activity of PRL-3 may therefore be used as a method of prognosis of an individual with cancer, such as leukemia.

PRL3 may act as an independent prognostic indicator. Thus, the level of PRL3 may be used to predict an individual's prognosis without requiring the analysis of other prognostic indicator molecules, or other prognostic indicator genes (i.e. genes other than PRL3 whose expression, activity or level is modulated in cancer). In some cases, the methods of prognosis described herein involve the prognosis of cancer is determined on the basis of PRL3, but not other prognostic indicator genes. In some cases, other prognostic Indicator genes are assessed in addition to PRL3.

PRL3 level may be calculated as a univariate analysis. That is to say, in the absence of analysis of any other prognostic indicators.

Alternatively, PRL3 may be used as part of a multivariate prognostic test, in which other prognostic factors are analysed, in addition to PRL3. PRL3 may be used as part of a multivariate prognostic model or prediction model.

Additional prognostic indicators may include the analysis of expression levels or activity of proteins or nucleic acids, such as oncogenes or known prognostic marker genes, expression of known mutant proteins, nucleic acids or genes, or other factors such as the age, sex, general health, symptoms, signs, test results or medical history of the patient. Clinical and non-clinical prognostic indicators will be readily appreciated to those of skill in the relevant art.

The prognosis may be for a sample or patient that has a normal karyotype. In some cases, the sample or patient may exhibit an abnormal karyotype, such as an abnormal number or structure of chromosomes or other cytogenetic complication.

The sample may be from a patient who has already been treated with an anti-cancer therapy, such as chemotherapy, radiotherapy or hormone treatment. In some cases, the cancer will not have responded to the anti-cancer therapy.

Prognosis may be used to predict the disease free survival time of an individual, progression-free survival time, disease specific survival time, survival rate, or survival time.

Survival rate (also known as overall survival) is the percentage of people who are alive for a given period of time after prognosis. For example, the percentage of people who are alive 1 month, 3 months, 6 months, 12 months, 18 months, 24 months, 3 years, 4, years, 5 years, 10 years or longer, after the prognosis is made. Survival rate may be a percentage likelihood that the patient will be alive in a particular period of time, for example, a 5%, 10%, 15%, 20%, 25%, 30%, 35%, 40%, 45%, 50%, 55%, 60%, 65%, 70%, 75%, 80%, 85%, 90%, 90% or 100% likelihood that a patient will be alive in 1 month, 3 months, 6 months, 12 months, 18 months, 24 months, 3 years, 4, years, 5 years, 10 years or longer.

Survival time is the remaining duration of life of the patient. For example an estimated time of 1 month, 3 months, 6 months, 12 months, 18 months, 24 months, 3 years, 4, years, 5 years, 10 years or longer time alive following the prognosis.

Disease free survival time (DFS) is the length of time after a primary treatment for a cancer ends that the patient survives without signs or symptoms of that cancer.

Progression-free survival time is the length of time during which the disease does not get worse.

The survival time may be a disease-specific survival time, which is the percentage of people who are alive for a given period of time after prognosis, treating deaths from other causes than the cancer as withdrawals from the population that don't lower survival, and thus comparable to patients who are not observed any longer (e.g. due to reaching the end of the study period).

A poor prognosis is a prediction that a disease, such as cancer, will recur or worsen. It may be an indication that the patient will die from the disease or cancer. A poor prognosis may be indicative of a more aggressive cancer. One or more of a short survival time, short survival rate, short disease free survival time, short progression-free survival time, short disease specific survival time are indicative of a poor prognosis.

Diagnosis

Diagnosis refers to the identification of a disease, such as cancer. Methods described herein may be used to detect a cancer. They may be used to diagnose a subtype or subclass of a particular cancer.

Detection in a sample of PRL3 polypeptides or nucleic acids in accordance with the methods of the present invention may be used for the purpose of diagnosis of a cancerous condition in the patient, diagnosis of a predisposition to a cancerous condition or for determining a prognosis (prognosticating) of a cancerous condition. The diagnosis or prognosis may relate to an existing (previously diagnosed) cancerous condition, which may be benign or malignant, may relate to a suspected cancerous condition or may relate to the screening for cancerous conditions in the patient (which may be previously undiagnosed).

Other diagnostic tests may be used in conjunction with those described here to enhance the accuracy of diagnosis or prognosis of a cancerous condition or to confirm a result obtained by using the tests described here.

The method of diagnosis may be an in vitro method performed on the patient sample, or following processing of the patient sample. Once the sample is collected, the patient is not required to be present for the in vitro method of diagnosis to be performed and therefore the method may be one which is not practised on the human or animal body.

Other diagnostic tests may be used in conjunction with those described here to enhance the accuracy of the diagnosis or prognosis or to confirm a result obtained by using the tests described here.

Detection and Quantification

Methods disclosed herein involve the detection and/or quantification of PRL3. Detection, as used herein, refers to measurement of PRL3 without quantification. Methods for detection and quantification of PRL3 nucleotides and proteins are well known in the art and will be readily appreciated by a skilled person.

Protein, for example, may be detected or quantified by immunoassay. Immunoassay methods are well known in the art and will generally comprise: (a) providing a polypeptide comprising an epitope bindable by an antibody against said protein; (b) incubating a biological sample with said polypeptide under conditions which allow for the formation of an antibody-antigen complex; and (c) determining whether antibody-antigen complex comprising said polypeptide is formed. Immunoassay methods include western blotting and ELISA.

Immunoassays include, but are not limited to, Enzyme-linked immunosorbant assay (ELISA), lateral flow test, latex agglutination, other forms of immunochromatography, western blot, and/or magnetic immunoassay.

Protein may also be detected or quantified using mass spectrometry. For example, mass spectrometry using electrospray ionization (ESI) or matrix-assisted laser desorption/ionisation (MALDI).

Other methods of protein quantification include spectroscopy based methods. Such methods may involve colorimetric assays or spectrophotometric assays.

Methods for detecting and quantifying nucleic acids are well known in the art. Methods include polymerase chain reaction (PCR) based methods and hybridization methods.

Polymerase chain reaction based methods include PCR, reverse transcription PCR (RT-PCR and quantitative RT-PCR. Such methods utilise a primer, or short DNA fragment which binds specifically to a DNA sequence of interest. RNA may be transcribed to DNA before or during the method.

Elevated Expression or Activity

As disclosed herein, elevated PRL3 expression is indicative of a poor prognosis for cancer patients. As used herein, elevated expression is used interchangeably with increased expression, or high expression.

Elevated expression means an increase in the level of PRL3 protein or nucleic acid. The expression may be elevated locally or globally, for example within a particular tissue or cell type, such as within a tumor or within bone marrow, or maybe elevated throughout the body of the patient. Elevated expression may be caused by an increase in production of that protein or nucleic acid, or by a decrease in the elimination or destruction of that protein or nucleic acid, or both.

Elevated activity may be caused by an increase in the amount of the protein or nucleic acid, or by an increase in the activity of each individual molecule. This may occur through a mutation in the gene or protein sequence, such as an activating mutation, or may be due to a post-translational change, such as aberrant protein phosphorylation.

In some cases, the expression of PRL3 is significantly upregulated in the patient or sample, relative to the expression in a non-cancerous individual or a non-cancerous tissue.

Overexpression or increased activity of PRL3 relative to a control is indicative of a poor prognosis and poor survival. Very high overexpression or very high activity of PRL3 is indicative of a very poor prognosis, and very poor survival.

In some cases, expression or activity of 5%, 10%, 15%, 20%, 25%, 30%, 35%, 40%, 45%, 50%, 55%, 60%, 65%, 70%, 75%, 80%, 85%, 90%, 95%, 100%, 150%, 200%, 300%, 400%, 500%, 750% or 1000% or a higher percentage more than the expression or activity in the control is indicative of a poor prognosis.

In some cases, expression or activity of 1.5 times, 2 times, 3 times, 4 times, 5 times, 6 times, 7 times, 8 times, 9 times, 10 times, 15 times, 20 times, 25 times, 30 times, 35 times, 40 times, 45 times, 50 times, 100 times, or more times more than the expression or activity in the control is indicative of a poor prognosis.

Control

In some cases, the method involves comparing PRL3 in a sample from a patient with PRL3 in one or more control samples.

The comparison may not require the analysis of the control sample to be simultaneously or sequentially performed with the analysis of the sample from the patient. Instead, the comparison may be made with results previously obtained from a control sample, such as results stored in a database.

The control sample may be a sample obtained from the patient prior to the onset of cancer, or prior to the observation of symptoms associated with cancer.

The control sample may be a sample obtained from another individual, such as an individual who does not have cancer. The individual may be matched to the patient according to one or more characteristics, for example, sex, age, medical history, ethnicity, weight or expression of a particular marker. The control sample may have been obtained from the bodily location, or be of the same tissue or sample type as the sample obtained from the patient.

The control sample may be a collection of samples, thereby providing a representative value across a number of different individuals or tissues.

Hybridization

Certain methods described herein involve nucleotide sequences that are capable of hybridizing selectively to any of the PRL3 sequences described herein or to the complement of any of the above. Nucleotide sequences may be at least 1 nucleotides in length, such as at least 20, 30, 40 or 50 nucleotides in length.

The term “hybridization” as used herein shall include “the process by which a strand of nucleic acid joins with a complementary strand through base pairing” as well as the process of amplification as carried out in polymerase chain reaction technologies.

Polynucleotides capable of selectively hybridizing to the nucleotide sequences presented herein, or to their complement, will be generally at least 70%, such as at least 80 or 90% and such as at least 95% or 98% homologous to the corresponding nucleotide sequences presented herein over a region of at least 20, such as at least 25 or 30, for instance at least 40, 60 or 100 or more contiguous nucleotides.

The term “selectively hybridizable” means that the polynucleotide used as a probe is used under conditions where a target polynucleotide is found to hybridize to the probe at a level significantly above background. The background hybridization may occur because of other polynucleotides present, for example, in the cDNA or genomic DNA library being screened. In this event, background implies a level of signal generated by interaction between the probe and a non-specific DNA member of the library which is less than 10 fold, such as less than 100 fold as intense as the specific interaction observed with the target DNA. The intensity of interaction may be measured, for example, by radiolabelling the probe, e.g. with ³²P.

Hybridization conditions are based on the melting temperature (Tm) of the nucleic acid binding complex, as taught in Berger and Kimmel (1987, Guide to Molecular Cloning Techniques, Methods in Enzymology, Vol 152, Academic Press, San Diego Calif.), and confer a defined “stringency” as explained-below.

The polynucleotides described here may be used to produce a primer, e.g. a PCR primer, a primer for an alternative amplification reaction, a probe e.g: labeled with a revealing label by conventional means using radioactive or non-radioactive labels, or the polynucleotides may be cloned into vectors. Such primers, probes and other fragments will be at least 15, such as at least 20, for example at least 25, 30 or 40 nucleotides in length, and are also encompassed by the term polynucleotides as used herein. Fragments may be less than 500, 200, 100, 50 or 20 nucleotides in length.

Polynucleotides such as a DNA polynucleotides and probes may be produced recombinantly, synthetically, or by any means available to those of skill in the art. They may also be cloned by standard techniques.

In general, primers will be produced by synthetic means, involving a step wise manufacture of the desired nucleic acid sequence one nucleotide at a time. Techniques for accomplishing this using automated techniques are readily available in the art.

Longer polynucleotides will generally be produced using recombinant means, for example using PGR (polymerase chain reaction) cloning techniques. This will involve making a pair of primers (e.g. of about 15 to 30 nucleotides) flanking a region of the sequence which it is desired to clone, bringing the primers into contact with mRNA or cDNA obtained from an animal or human cell, performing a polymerase chain reaction under conditions which bring about amplification of the desired region, isolating the amplified fragment (e.g. by purifying the reaction mixture on an agarose gel) and recovering the amplified DNA. The primers may be designed to contain suitable restriction enzyme recognition sites so that the amplified DNA can be cloned into a suitable cloning vector.

Cancer

A “cancer” can comprise any one or more of the following: acute lymphocytic leukemia (ALL), acute myeloid leukemia (AML), adrenocortical cancer, anal cancer, bladder cancer, blood cancer, bone cancer, brain tumor, breast cancer, cancer of the female genital system, cancer of the male genital system, central nervous system lymphoma, cervical cancer, childhood rhabdomyosarcoma, childhood sarcoma, chronic lymphocytic leukemia (CLL), chronic myeloid leukemia (CML), colon and rectal cancer, colon cancer, endometrial cancer, endometrial sarcoma, esophageal cancer, eye cancer, gallbladder cancer, gastric cancer, gastrointestinal tract cancer, hairy cell leukemia, head and neck cancer, hepatocellular cancer, Hodgkin's disease, hypopharyngeal cancer. Kaposi's sarcoma, kidney cancer, laryngeal cancer, leukemia, leukemia, liver cancer, lung cancer, malignant fibrous histiocytoma, malignant thymoma, melanoma, mesothelioma, multiple myeloma, myeloma, nasal cavity and paranasal sinus cancer, nasopharyngeal cancer, nervous system cancer, neuroblastoma, non-Hodgkin's lymphoma, oral cavity cancer, oropharyngeal cancer, osteosarcoma, ovarian cancer, pancreatic cancer, parathyroid cancer, penile cancer, pharyngeal cancer, pituitary tumor, plasma cell neoplasm, primary CNS lymphoma, prostate cancer, rectal cancer, respiratory system, retinoblastoma, salivary gland cancer, skin cancer, small intestine cancer, soft tissue sarcoma, stomach cancer, stomach cancer, testicular cancer, thyroid cancer, urinary system cancer, uterine sarcoma, vaginal cancer, vascular system, Waldenstrom's macroglobulinemia and Wilms' tumor.

Cancers may be of a particular type. Examples of types of cancer include astrocytoma, carcinoma (e.g. adenocarcinoma, hepatocellular carcinoma, medullary carcinoma, papillary carcinoma, squamous cell carcinoma), glioma, lymphoma, medulloblastoma, melanoma, myeloma, meningioma, neuroblastoma, sarcoma (e.g. angiosarcoma, chrondrosarcoma, osteosarcoma).

In certain preferred embodiments, the cancer to be prognosed is leukemia, more preferably acute myeloid leukemia.

The cancer or leukemia is a PRL3 expressing cancer or leukemia. That is, a PRL3 positive cancer or leukemia.

Leukemia

Leukemia (leukaemia) is a type of cancer of the blood or bone marrow characterized by an abnormal increase of immature white blood cells called blasts. Treatment of leukemia involves chemotherapy, medical radiation therapy, or hormone treatments.

Clinically and pathologically leukemia is subdivided into a variety of large groups. Acute leukemia is characterized by a rapid increase in the number of immature blood cells. Chronic leukemia is characterized by the excessive build-up of relatively mature, but still abnormal, white blood cells.

In lymphoblastic leukemia or lymphocytic leukemia the cancerous change takes place in a type of marrow cell that normally goes on to form lymphocytes, usually B cells. In myeloid or myelogenous leukemias the cancerous change takes place in a type of marrow cell that normally goes on to form red blood cells, some other types of white cells, and platelets.

As used herein, the term “leukemia” includes myeloid and lymphocytic leukemia. Thus, leukemia includes acute myeloid leukemia, chronic myeloid leukemia, acute lymphoblastic leukemia, chronic lymphocytic leukemia, hairy cell leukemia, T-cell prolymphocytic leukemia, large granular lymphocytic leukemia and adult T-cell leukemia.

Acute Myeloid Leukemia

Acute Myeloid Leukemia (AML) is also known as acute myelogenous leukemia or acute non-lymphocytic leukemia (ANLL). It is a cancer of the myeloid line of blood cells, characterized by the rapid growth of abnormal white blood cells that accumulate in the bone marrow and interfere with the production of normal blood cells. AML is the most common acute leukemia affecting adults, and its incidence increases with age. Although AML is a relatively rare disease, accounting for approximately 1.2% of cancer deaths in the United States, its incidence is expected to increase as the population ages.

The symptoms of AML are caused by replacement of normal bone marrow with leukemic cells, which causes a drop in red blood cells, platelets, and normal white blood cells. These symptoms include fatigue, shortness of breath, easy bruising and bleeding, and increased risk of infection.

The first clue to a diagnosis of AML is typically an abnormal result on a complete blood count, whilst an excess of abnormal white blood cells (leucocytosis) is a common finding. Presumptive diagnosis of AML can be made via examination of the peripheral blood smear when there are circulating leukemic blasts, a definitive diagnosis usually requires an adequate bone marrow aspiration and biopsy. Marrow or blood is examined, for example by light microscopy or flow cytometry, to diagnose the presence of leukemia, to differentiate AML from other types of leukemia, and to classify the subtype of the disease. A sample is typically also tested for chromosomal abnormalities.

Chronic Myeloid Leukemia

Chronic myeloid or myelogenous leukemia (CML) is also known as chronic granulocytic leukemia (CGL). It is a form of leukemia characterized by the increased and unregulated growth of predominantly myeloid cells in the bone marrow and the accumulation of these cells in the blood. CML is a clonal bone marrow stem cell disorder in which a proliferation of mature granulocytes (neutrophils, eosinophils and basophils) and their precursors is found. It is a type of myeloproliferative disease associated with a characteristic chromosomal translocation called the Philadelphia chromosome. CML is now largely treated with targeted drugs called tyrosine kinase inhibitors (TKIs), such as Gleevec/Glivec (imatinib), Sprycel (dasatinib), Tasigna (nilotinib), or Bosulif (bosutinib) which have led to dramatically improved long term survival rates (95.2%) since the introduction of Gleevec in 2001. These drugs have revolutionized treatment of this disease and allow most patients to have a good quality of life when compared to the former chemotherapy drugs.

The inventors have determined, through analysis of the Gene Expression Atlas (http://www.ebi.ac.uk/gxa/gene/ENSG00000184489) that the expression level of PRL-3 was the highest in chronic myeloid leukemia 2 (CML) among 950 human cancer cell lines covering 32 different types of cancers (Dataset code: E-MTAB-37), suggesting a potential role of PRL-3 in CML pathogenesis as well.

FLT3

Fms-like tyrosine kinase 3 (FLT-3) is also known as cluster of differentiation antigen 135 (CD135). FLT3 is a cytokine receptor which belongs to the receptor tyrosine kinase class III. It is expressed on the surface of many hematopoietic progenitor cells. Signalling of Flt3 is important for the normal development of hematopoietic stem cells and progenitor cells. FLT3 may have a sequence according to the following Unigene references:

| Unigene | Version | Description |
|---|---|---|
| Z26652.1 | GI:406322 | Homo sapiens mRNA for FLT3 receptor |
| | | tyrosine kinase precursor (FLT3 gene) |
| NM_004119.2 | GI:121114303 | Homo sapiens fms-related tyrosine |
| | | kinase 3 (FLT3), mRNA |
| NP_004110.2 | GI:121114304 | Receptor-type tyrosine-protein kinase |
| | | FLT3 [Homo sapiens] |

Activating mutations in FLT3 are one of the more frequent genetic alterations in AML, involving internal tandem duplication (ITD) in the juxtamembrane (JM) domain of FLT3 (Nakao et al., 1996). The constitutive activation of FLT3-ITD leads to elevated and sustained activation of multiple downstream signalling pathways, ultimately resulting in the transformation of hematopoietic cells to growth factor-independent proliferation (Mizuki et al., 2000). High levels of wild-type FLT3 have also been reported for blast cells of some AML patients without FLT3 mutations Due to their essential pro-proliferative and anti-apoptotic roles in AML cells, activating mutations in FLT3 have been proposed as a promising molecular target for the treatment of AML.

As disclosed herein, PRL3 is an indicator of poor prognosis in patients with FLT3-ITD mutations. Thus, in some cases described herein, the method is carried out on a sample obtained from a patient with a FLT3-ITD positive AML. The method may involve determining whether the sample is a FLT3-ITD positive sample. The patient may have been previously determined to be FLT3-ITD positive.

Linifanib (ABT-869) is an aminobenzopyrazole-based ATP-competitive receptor tyrosine kinase inhibitor. It has been identified as an FLT3 inhibitor. It is proposed for treatment of a range of cancers, such as AML, colorectal cancer and non-small cell lung cancer. Other therapeutic agents based on FLT3 inhibition include sunitinib (SU11248, Sutent™), lestaurtinib (rINN, CEP-701) and Quizartinib (AC220).

In some cases described herein, the patient has received FLT3 inhibitory therapy. The patient may have received other therapies too, for example treatment with SAHA (suberoylamilide hydroxanic acid), a histone deacetylase inhibitor. In some cases, the patient has not responded to that therapy, or has only partially responded. Thus, the therapy may not have resulted in a reduction or elimination of the cancer or its symptoms.

Therapy

Described herein are methods of treatment, including methods of treatment of leukemia and method of treatment using anti-PRL3 antibodies. Agents for use in those methods, including anti-PRL3 agents and anti-PRL3 antibodies are also disclosed, along with the use of such agents in the manufacture of a medicament for the cancer.

The methods and compositions described here suitably enable an improvement in a measurable criterion in an individual to whom the treatment is applied compared to one who has not received the treatment. By the term “treatment” we mean to also include prophylaxis or alleviation of cancer.

Methods of treatment disclosed herein may be for the treatment of cancer, such as the treatment of leukemia. The treatment may result in an alleviation of the symptoms of the cancer, or may result in the complete treatment of the cancer. The treatment may slow the progression of the cancer, or may prevent the worsening of the symptoms of the cancer.

Also disclosed are medicaments comprising the agents useful in the methods of treatment disclosed herein. Medicaments may comprise anti-PRL3 agents such as anti-PRL3 antibodies.

Medicaments and pharmaceutical compositions according to aspects of the present invention may be formulated for administration by a number of routes, including but not limited to, parenteral, intravenous, intra-arterial, intramuscular, intratumoural, oral and nasal. The medicaments and compositions may be formulated in fluid or solid form. Fluid formulations may be formulated for administration by injection to a selected region of the human or animal body.

Administration is preferably in a “therapeutically effective amount”, this being sufficient to show benefit to the individual. The actual amount administered, and rate and time-course of administration, will depend on the nature and severity of the disease being treated. Prescription of treatment, e.g. decisions on dosage etc, is within the responsibility of general practitioners and other medical doctors, and typically takes account of the disorder to be treated, the condition of the individual patient, the site of delivery, the method of administration and other factors known to practitioners. Examples of the techniques and protocols mentioned above can be found in Remington's Pharmaceutical Sciences, 20th Edition, 2000, pub. Lippincott, Williams & Wilkins.

A treatment may involve administration of more than one therapeutic agent. An agent may be administered alone or in combination with other treatments, either simultaneously or sequentially dependent upon the condition to be treated. For example, the treatment may be a co-therapy involving administration of two agents, one or more of which may be intended to treat the cancer. Thus, anti-PRL3 antibody may be administered with another drug, such as a chemotherapeutic agent, prodrug, antibody or hormone treatment. The treatment may additionally involve radiotherapy.

Anti-PRL3 Antibodies

Antibodies which will bind to PRL3 are already known. For example, as disclosed in Jie Li et al., 2005.

The antigen-binding portion may be a part of an antibody (for example a Fab fragment) or a synthetic antibody fragment (for example a single chain Fv fragment [ScFv]). Suitable monoclonal antibodies to selected antigens may be prepared by known techniques, for example those disclosed in “Monoclonal Antibodies: A manual of techniques”, H Zola (CRC Press, 1988) and in “Monoclonal Hybridoma Antibodies: Techniques and Applications”, J G R Hurrell (CRC Press, 1982). Chimeric antibodies are discussed by Neuberger et al (1988, 8th International Biotechnology Symposium Part 2, 792-799).

Monoclonal antibodies (mAbs) are useful in the methods of the invention and are a homogenous population of antibodies specifically targeting a single epitope on an antigen. Suitable monoclonal antibodies can be prepared using methods well known in the art (e.g. see Köhler, G.; Milstein, C. (1975). “Continuous cultures of fused cells secreting antibody of predefined specificity”. Nature 256 (5517): 495; Siegel D L (2002). “Recombinant monoclonal antibody technology”. Schmitz U. Versmold A, Kaufmann P, Frank H G (2000); “Phage display; a molecular tool for the generation of antibodies—a review”. Placenta. 21 Suppl A: S106-12. Helen E. Chadd and Steven M. Chamow; “Therapeutic antibody expression technology,” Current Opinion in Biotechnology 12, no. 2 (Apr. 1, 2001): 188-194; McCafferty, J.; Griffiths, A.; Winter, G.; Chiswell, D. (1990). “Phage antibodies: filamentous phage displaying antibody variable domains”. Nature 348 (6301): 552-554; “Monoclonal Antibodies: A manual of techniques”, H Zola (CRC Press, 1988) and in “Monoclonal Hybridoma Antibodies: Techniques and Applications”. J G R Hurrell (CRC Press, 1982). Chimeric antibodies are discussed by Neuberger et al (1988, 8th International Biotechnology Symposium Part 2, 792-799)).

Polyclonal antibodies are useful in the methods of the invention. Monospecific polyclonal antibodies are preferred. Suitable polyclonal antibodies can be prepared using methods well known in the art.

Fragments of antibodies, such as Fab and Fab₂ fragments may also be used as can genetically engineered antibodies and antibody fragments. The variable heavy (V_{H}) and variable light (V_{L}) domains of the antibody are involved in antigen recognition, a fact first recognised by early protease digestion experiments. Further confirmation was found by “humanisation” of rodent antibodies. Variable domains of rodent origin may be fused to constant domains of human origin such that the resultant antibody retains the antigenic specificity of the rodent patented antibody (Morrison et al (1984) Proc. Natl. Acad. Sci. USA 81, 6851-6855).

That antigenic specificity is conferred by variable domains and is independent of the constant domains is known from experiments involving the bacterial expression of antibody fragments, all containing one or more variable domains. These molecules include Fab-like molecules (Better et al (1988) Science 240, 1041); Fv molecules (Skerra et al (1988) Science 240, 1038); single-chain Fv (ScFv) molecules where the V_{H} and V_{L} partner domains are linked via a flexible oligopeptide (Bird et al (1988) Science 242, 423; Huston et al (1988) Proc. Natl. Acad. Sci. USA 85, 5879) and single domain antibodies (dAbs) comprising isolated V domains (Ward et al (1989) Nature 341, 544). A general review of the techniques involved in the synthesis of antibody fragments which retain their-specific binding sites is to be found in Winter & Milstein (1991) Nature 349, 293-299.

By “ScFv molecules” we mean molecules wherein the V_{H} and V_{L} partner domains are covalently linked, e.g. directly, by a peptide or by a flexible oligopeptide. Fab, Fv, ScFv and dAb antibody fragments can all be expressed in and secreted from E. coli, thus allowing the facile production of large amounts of the said fragments.

Whole antibodies, and F(ab′)₂ fragments are “bivalent”. By “bivalent” we mean that the said antibodies and F(ab′) fragments have two antigen combining sites. In contrast, Fab, Fv, ScFv and dAb fragments are monovalent, having only one antigen combining site. Synthetic antibodies which bind to PRL3 may also be made using phage display technology as Is well known in the art (e.g. see “Phage display: a molecular tool for the generation of antibodies—a review”. Placenta. 21 Suppl A: S106-12. Helen E. Chadd and Steven M. Chamow; “Phage antibodies: filamentous phage displaying antibody variable domains”. Nature 348 (6301): 552-554).

In some preferred embodiments the antibody is detectably labelled or, at least, capable of detection. For example, the antibody may be labelled with a radioactive atom or a coloured molecule or a fluorescent molecule or a molecule which can be readily detected in any other way. Suitable detectable molecules include fluorescent proteins, luciferase, enzyme substrates, and radiolabels. The antibody may be directly labelled with a detectable label or it may be indirectly labelled. For example, the antibody may be unlabelled and can be detected by another antibody which is itself labelled. Alternatively, the second antibody may have bound to it biotin and binding of labelled streptavidin to the biotin is used to indirectly label the first antibody.

Antibodies disclosed herein bind to PRL-3. PRL3 is an intracellular oncoprotein. Thus, the anti-PRL3 antibodies according to the invention may be capable of entering cells. For example, the antibodies may be capable of crossing the plasma membrane to bind PRL3 within the cell, in an intracellular environment. The antibodies may inhibit a biological activity of PRL3, such as protein tyrosine phosphatase (PTP) activity.

The antibody may be an antibody capable of binding epitope KAKFYN and/or HTHKTR. The antibody may be an antibody having a sequence identical to mouse anti-PRL3 antibody from hybridoma clone 223 or hybridoma clone 318, as reported by Li et a 2005. The antibody may compete for target binding with the antibody from hybridoma clone 223 or hybridoma clone 318 described in Li et al 2005. The antibody may be an antibody having a heavy chain and/or a light chain variable domain sequence as set out in FIG. 16. The antibody may be a humanised antibody, a chimeric antibody or a fully human antibody. The antibody be homologous to an antibody described herein.

As indicated above, with respect to sequence identity, a “homologue” has such as at least 5% identity, at least 10% identity, at least 15% identity, at least 20% identity, at least 25% identity, at least 30% identity, at least 35% identity, at least 40% identity, at least 45% identity, at least 50% identity, at least 55% identity, at least 80% identity, at least 65% identity, at least 70% identity, at least 75% identity, at least 80% identity, at least 82% identity, at least 84% identity, at least 86% identity, at least 88% identity, at least 90% identity, at least 92% identity, at least 94% identity, at least 96% identity, or at least 98% identity to a relevant sequence. The relevant sequence may be the CDR sequence, or across the sequence of the heavy and/or light variable chain.

Aspects and embodiments of the present invention will now be illustrated, by way of example, with reference to the accompanying figures. Further aspects and embodiments will be apparent to those skilled in the art.

### EXAMPLES

Materials and Methods

Cell Lines and Primary Patient Samples

TF-1 and MV4-11 cells were purchased from ATCC (American type culture collection, Manassas, Va.). MOLM-14 cell line was obtained in house. TF-1 cells were cultured in RPMI 1640 (Invitrogen, Carlsbad, Calif.) supplemented with heat-inactivated 10% fetal bovine serum (Hyclone Laboratories, Inc., Logan, Utah) and supplemented with 2 ng/ml human IL-3 (R & D system Inc., Minneapolis, Minn.). TF1-ITD and TF1-PRL-3 cells were prepared as described previously (Kim et al, 2005; Zhou et al, 2011). Bone marrow (BM) blast cells were obtained from newly diagnosed AML patients with written informed consent from National University Hospital. Singapore. This study was approved by institutional Review Board (IRB) of National University of Singapore.

Chemicals and Reagents

FLT3 inhibitor (PKC412), MEK inhibitor (U0126), p38 MAPK inhibitor (SB203580), and JNK inhibitor (SP600125) were purchased from LC Laboratories (Woburn, Mass.). FLT3 inhibitor (CEP-701) and Src kinase inhibitors (SU6656 and PP2) were purchased from Sigma (St. Louis, Mo.). Antibodies to FLT3, STAT5, JAK2, Src, ERK, c-Jun, pFLT3, pJAK2, pSTAT5, pSrc and p-c-Jun were purchased from Cell Signalling Technologies (Beverly, Mass.). GAPDH antibody was obtained from Millipore (Billerica, Mass.). Anti-CD45-APC and LightShift Chemiluminescent EMSA Kit were from Pierce Biotechnology. Inc. (Rockford, Ill.). Mouse anti-PRL3 antibody was from hybridoma clone 318 as reported previously (Li et al. 2005). Secreted alkaline phosphatase (SEAP) reporter assay Kit was purchased from Clontech (Palo Alto, Calif.) and Phospha-Light™ from Applied Biosystems (Bedford, Mass.).

Detection of FLT3-ITD Mutation and Expression of PRL-3 by RT-PCR

Total RNA was extracted from AML patients' bone marrow cells using RNeasy minikit (Qiagen, Chatsworth, Calif.) according to the manufacturer's instructions. cDNA was synthesized from total RNA by reverse transcriptase III (Invitrogen, Carlsbad, Calif.) and amplified by PCR as described before (Quentmeier et al, 2003). The primer sets for RT-PCR were summarized; FLT3-ITD, 5′-GCAATTTAGGTATGAAAGCCAGC-3′ and 5′-CTTCAGCATTTTGACGGCAACC-3′, PRL-3, 5′-GGGACTTCTCAGGTCGTGTC-3′ and 5′-AGCCCCGTACTTCTTCAGGT-3′, and the O-actin gene was 5′-GTGGGGCGCCCCAGGCACCA-3′ and 5′-CTCCTTAATGTCACGCACGATTTC-3′. The PCR products were analyzed on a 5% polyacrylamide gel, stained with ethidium bromide, and then visualized with GelDoc imager (BioRad Inc, Hercules, Calif.).

Quantitative Real Time PCR

Quantitative real time PCR (Q-RT-PCR) was used to measure the mRNA expression levels of PRL-3 at human BM samples and AML cell lines (ABI 7500 Fast Real Time PCR system). The cDNAs were served as template for Q-RT-PCR by using TaqMan® Universal PCR Master Mix kit (Applied Biosystems, Foster City, Calif.). Each 10 μl of quantitative PCR reaction mixture contained 5 μl of 2× TaqMan® Universal Master Mixture (Applied Biosystems), 4.5 μl of diluted cDNA mixture, and 0.5 μl of gene specific probe. To standardize the quantification of the selected target genes, GAPDH served as internal controls and were quantified on the same plate as the target genes.

Western Blot Analysis

Cells were lysed using modified RIPA buffer (50 mM Tris-HCl, 150 mM NaCl, 1% NP-40, pH 8.0, 1×protease inhibitor cocktail) and lysates were subjected to western blotting with indicated primary antibodies. Proteins recognized by the antibodies were detected using the Chemiluminescent Detection Kit (Pierce, Thermo Scientific, Rockford, Ill.).

Transient Transfection of siRNA or Reporter Vector

TF-1, TF1-ITD, MOLM-14; or MV4-11 cells were re-suspended at 2×10⁶ cells per 100 μL of appropriate Nucleofector kit solution (Amaxa Biosystems, Cologne, Germany) and were nucleofected with 2 μg of FLT3 SMARTpool siRNA duplexes (Dharmacon Research, Milipore), PRL-3 siRNA, Signal Silence STAT5 siRNA I/II (Cell Signalling Technologies), AP-1 SEAP reporter vector, ERK siRNA, JNK siRNA or non-silencing siRNA (Santa Cruz Biotechnology, CA). After nucleofection, the cells were immediately mixed with 500 μL of pre-warmed culture medium and transferred to culture plates for incubation. Samples were collected for protein extraction as described above.

Electrophoretic Mobility Shift Assay (EMSA)

The Transcription Factor Database (TRANSFAC) (Wingender et al, 1996) was used to predict possible transcription factor binding sites on human PRL-3 promoter region. Nuclear extracts were prepared using NE-PER nuclear protein extraction kit (Pierce, Thermo Scientific). For EMSA probes, 30 bp long, complementary sense and antisense strands of DNA oligonucleotides were annealed and diluted to 50 fmol/μl. DNA probes (50 fmol/μl) and nuclear extracts were mixed with EMSA buffer 150 mM MgCl2, 1% glycerol, 0.01% NP-40, 1 mM DTT, and 0.1 mg/ml poly (dI-dC)] and incubated at room temperature for 30 min. In the competition reaction, unlabelled competitor was added in at least 10:1 molar excess over the biotinylated probe. Reaction mixtures were run on a native gel and visualised by LightShift chemiluminescent EMSA Detection kit. The probe sequences (sense strand) used in this study include; S1, 5′-GGTGATGTTTTCTGGAAGTGTGGGT-3′, S2, 5′-CCATAAGTTCTTGGAAGCTGCGGCTT-3′ and STAT5 competitor sequence, 5′-AGATTTCTAGGAATTCAATCC-3′.

Luciferase Reporter Assay

A −5.4 kb upstream region of PRL-3 (−5556 to −5331, S1a, numbered from a transcription initiation site) and its 5′-sequential deletion fragments (−5472 to −5331, −5440 to −5331, and −5399 to −5331; S1b, S1c, and S1d, respectively) were subcloned into the pGL-Luc-basic vector. STAT5A and STAT5B expression vectors were purchased from OriGene Technologies, Inc (Rockville, Md.). TF-1 cells were seeded in 6 well plate and transfected with an expression vector of STAT5A or STAT5B along with 1.5 μg of an appropriate luciferase reporter construct by nucleofection method (Lonza Cologne AG, Switzerland). Luciferase assays were performed using Dual-Luciferase Reporter system (Promega, Madison, Wis.), in which relative firefly luciferase activities were calculated by normalizing transfection efficiency according to the renilla luciferase activities. The expression level of STAT5A or STAT5B was determined by western blotting analysis.

Secreted Alkaline Phosphatase (SEAP) Assay

The AP-1 reporter vector (AP1-SEAP) was purchased from Clontech. TF1-GFP cells and TF1-PRL-3 cells were transfected with 200 ng of AP1-SEAP vector and incubated for 24 hrs. The culture supernatant was collected, heated at 65° C. for 30 min, and assayed for alkaline phosphatase activity as follows; 30 μl of supernatant was incubated with 120 μl of assay buffer for 5 min, at which time 1:20 diluted CSPD substrate was added, and samples were read on a TECAN microplate reader (Maennedorf, Switzerland).

Cell Viability Assay

AML Cells (1×10⁴) were seeded into each well of 96-well tissue culture plates in 100 μl growth media and viable cells were measured after seeding with different inhibitors for 72 hr using the CellTiter⁹⁶ Aqueous cell proliferation assay kit. (Promega). Briefly, an aliquot of 20 μl MTS mixture was added at the indicated time of assay and reactions were performed at 37° C. for 2 hours. And absorbance was read at 490 nm wavelength using TECAN microplate reader. After establishment of linear relationship between cell numbers and absorbance from each cell line, acquired absorbance converted to cell number.

Cell Cycle Analysis

The DNA content of cultured cells was quantitated by staining with propidium iodide (PI) and analyzed by flow cytometry (BDLSR11, Becton Dickinson, San Jose, Calif.). Briefly, Cells were harvested with PBS and fixed with cold 70% ethanol at 4° C. for 30 min. The cells were washed with PBS and then resuspended in 500μ of PI staining solution and incubated for 30 min at room temperature. Samples were then examined and analyzed for cell cycle phase (Modfit LT2.0, Becton Dickinson).

Annexin-V and 7-Aminoactinomycin D (7-AAD) Staining

TF1-GFP and TF1-PRL-3 cells were harvested with PBS after 48 hr culture in the lack of cytokine supplement. The cells were washed with PBS twice and incubated with annexin-V and 7-AAD staining solution for 30 min at room temperature. After staining, cells were subjected to FACS analysis.

Cell Line Generation

To generate PRL-3 knock-down cell lines, pRS-PRL-3-shRNA (OriGene Technologies, Rockville, Md.) was transfected into TF1-ITD cells. The resulting PRL-3-KD cell lines were selected with puromycin, and confirmed by western blot analysis. TF1-ITD PRL-3-KD cells were used for mice injection.

Anti-Leukemic Effects in Mouse Model

All animal studies as described previously (Guo et al, 2008) have been approved by Institutional Animal Care and Use Committee (IACUC). We followed the policies from the Animal Facility Center of The Agency for Science, Technology and Research (A* STAR), Singapore. Balb/c nude mice were obtained from Biological Resource Center (A* STAR, Singapore). Nude mice were intravenously injected with 1×10⁶ TF1-ITD cells. Three days later, mice were randomly divided into three treatment groups: injected twice weekly with IgG (sham treated, n=11), PRL-3 mAb (PRL-3 treated, n=11), and FLT3 mAb (FLT3 treated, n=1). For survival study, mice treated with IgG (n=7) or -treated with PRL-3 mAb (n=7) were used and observed daily. Mice injected with TF-1 cells were used as controls for mice injected with TF1-ITD cells. Organs were isolated and inspected for macroscopic metastases at the end of the experiments (Guo et al, 2008).

Human Leukemic Cells Engraftment Analysis

Nude mice were intravenously injected with 1×10⁶ TF-1, TF1-ITD, or TF1-ITD PRL-3 KD cells. Mice injected with TF1-ITD cells were divided into two groups and bi-weekly treated with control IgG or PRL-3 mAb. At the end of experiments, bone marrow cells were isolated and stained with human specific hematopoietic cell surface marker. CD45-APC antibody (Pierce, Thermo Scientific), and analysed by flow cytometry.

Analysis of AML Cancer Patient Microarray Data

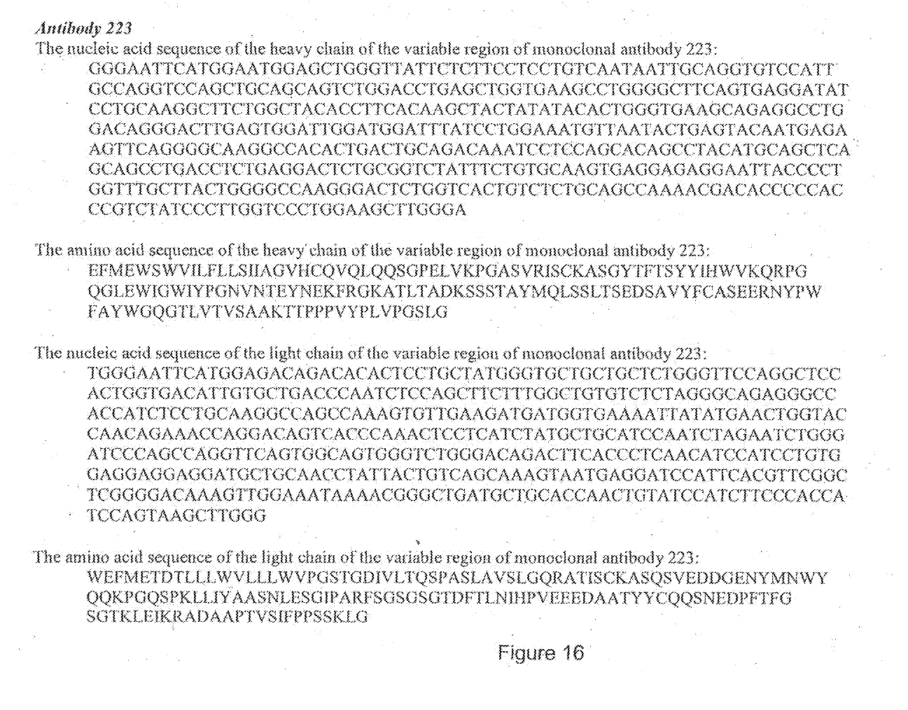

Details of all human AML patients datasets used in this study were summarized below. A total of 5 independent AML patient datasets were analyzed: 1) Our unpublished dataset (Cohort 1), analyzed on Affymetrix U133Ptus2 arrays from Belfast, UK.; 2) GEO-accessible GSE1159 dataset (Valk et at, 2004); 3) GEO-accessible GSE6891 dataset (Verhaak et al, 2009); 4) GEO-accessible GSE15434 dataset (Kohlmann et al, 2010); and 5) GEO-accessible GSE12417 dataset (Metzeler et al, 2008). FIG. 10 shows the information available in each dataset, and the figure numbers in this report where the raw data were used. Datasets were pre-processed using R and Bioconductor for normalization. The median value was used as a cut-off point to differentiate high and low levels of PRL-3 (Average of two PRL-3 probes; 206574 and 209695). Statistical analyses were performed using SPSS19.0 (IBM, Singapore). Correlation between PRL-3 expression and FLT3-ITD mutation status was analyzed by Fisher exact test or Chi-square test where applicable. The association between PRL-3 expression and survival time was analyzed by Kaplan-Meier analysis compared by log-rank test, p<0.05 was considered significant. Cox-regression analysis with backward conditional stepwise selection with a removal limit of p>0.05 was performed to identify independent predictors for AML patient survival.

PRL-3 is Frequently Upregulated in AML Patients with FLT3-ITD Mutations

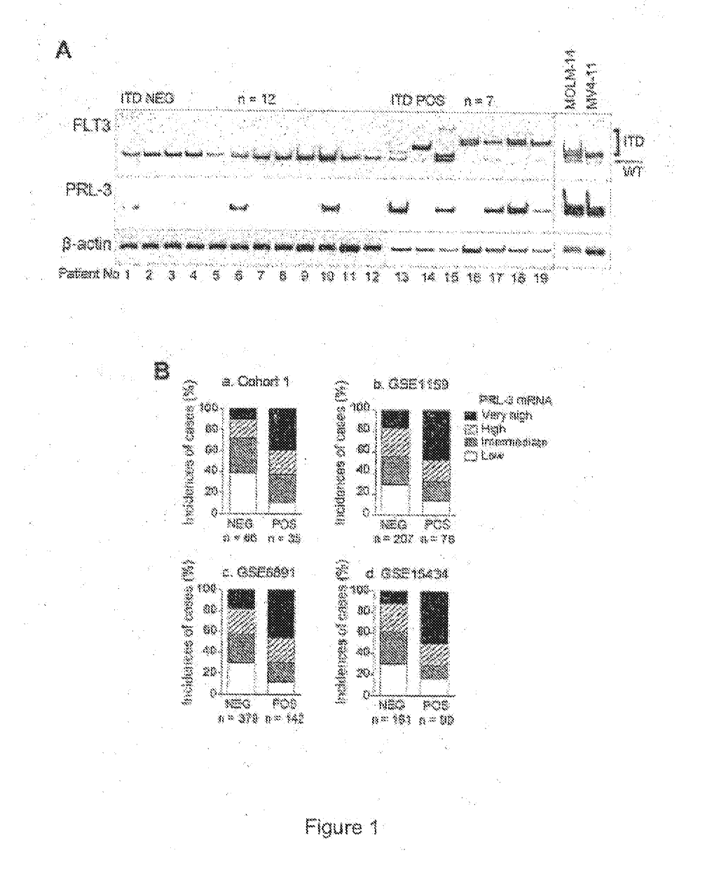

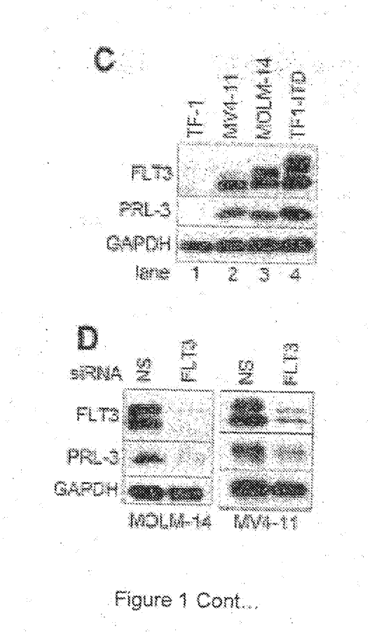

To investigate a correlation between PRL-3 overexpression and FLT3-ITD mutations, 19 bone marrow samples from AML patients with or without FLT3-ITD mutations were analysed. The incidence of PRL-3 upregulation in AML was found to be significantly associated with FLT3-ITD mutation (5 out of 7 cases, 71.4%), compared with only 3 out of 12 (25%) cases without FLT3-ITD mutation (Fisher's exact test, p<0.05; FIG. 1A). Similarly, high PRL-3 expression was observed in two FLT3-ITD positive cell lines (MOLM-14 and MV4-11) (FIG. 1A). Quantitative real-time PCR analysis of PRL-3 mRNA from the same patients supported that a higher PRL-3 mRNA expression was associated with AML patients with FLT3-ITD mutations (FIG. 11). To extend this finding, we analysed our unpublished Belfast/MILE dataset (Cohort 1), consisting of total 221 AML patients. Among them, 101 patients with normal karyotype were used to analyse the relationship between PRL-3 expression level and FLT3-ITD mutation status. Only 10% of FLT3-ITD negative AML patients expressed “very highly” PRL-3 (Chi-square test, p<0.001), whereas over 40% of FLT3-ITD positive patients expressed “very highly” PRL-3 (black block, FIG. 1B, a). Our observation was further corroborated in three independent, publicly available AML patient datasets (GSE1159 n=285, GSE6891 n=521, and, GSE15434 n=251), where PRL-3 expression was consistently observed to be significantly higher in AML patients who were positive for FLT3-ITD mutation compared to those who were negative for FLT3-ITD mutations in three independent datasets (FIG. 1B, b-d; p<0.001). In summary, our analysis of four separate AML patient cohorts show a strong association between FLT3-ITD mutations and high PRL-3 expression in a total of 1158 AML patients.

These results indicate that constitutive activation of FLT3 signalling might lead to PRL-3 overexpression in AML patients. To validate the clinical data, we either overexpressed or depleted FLT3-ITD in human myeloid leukemia cell lines. Compared with TF-1 control cells (FIG. 1C, lane 1), both MV4-11 and MOLM-14 cell lines harbouring endogenous FLT3-ITD mutations and TF-1 cell line over-expressing exogenous FLT3-ITD (TF1-ITD) had higher levels of PRL-3 (FIG. 1C, lanes 2-4). In contrast, siRNA-mediated depletion of FLT3 expression in MOLM-14 and MV4-11 cells effectively suppressed PRL-3 expression (FIG. 1D). Collectively, our results allude to a close relationship between FLT3-ITD mutation and elevated PRL-3 expression in AML cells.

Constitutive Activation of FLT3 Enhances PRL-3 Expression Through Src-STAT5 Signalling Pathway

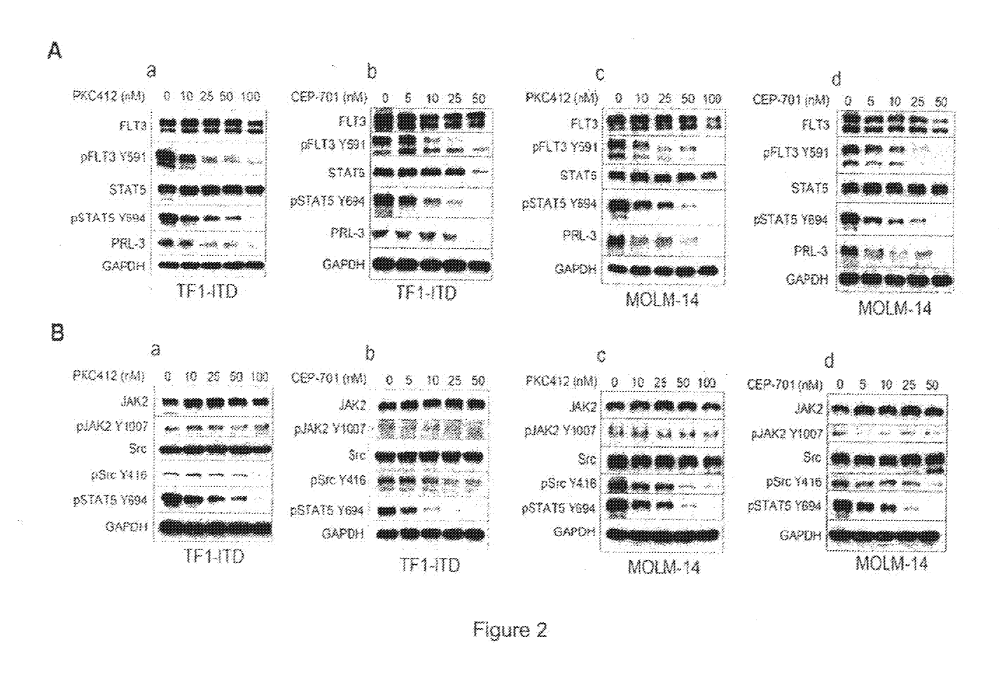

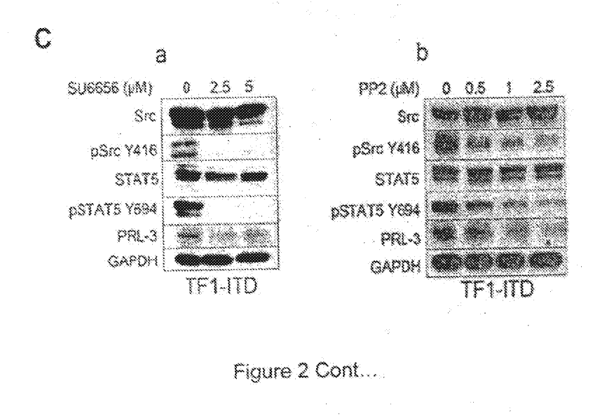

To investigate if constitutively active FLT3 signalling was involved in upregulation of PRL-3 expression, we used FLT3 inhibitors to block FLT3 receptor activity and examined the downstream signalling molecules of FLT3-ITD mutation. Since STAT5 was known to be a critical downstream target of FLT3-ITD (Mizuki et al, 2000), we tested STAT5 expression level after treatment with FLT3-specific inhibitors; PKC412 or CEP-701 (Odgerel et al, 2007; Smith et al, 2004). The respective inhibitors reduced phosphorylation of FLT3 and STAT5 in a dose dependent manner and resulted in a corresponding decrease in PRL-3 protein levels in TF1-ITD and MOLM-14 cell lines (FIG. 2A). We next examined whether FLT3-ITD-induced PRL-3 expression might be mediated by JAK or Src, two distinct upstream activators of STAT5 (Robinson et al, 2005; Spiekermann et al, 2003). After treatment with FLT3 inhibitors, both phospho- and total-JAK2 levels were not affected (FIG. 2B), whereas the activated form of Src (pSrc Y416) was potently down-regulated after treatment. Importantly. Src inactivation closely corresponded with a decrease of STAT5 phosphorylation in a dose-dependent manner (FIG. 2B). To investigate the role of Src-mediated phosphorylation of STAT5 in FLT3-ITD positive AML cells. AML cells were treated with two distinct Src kinase inhibitors, SU6656 and PP2 (Blake et al, 2000; Nam et al, 2002). Src inhibition reduced both STAT5 phosphorylation and PRL-3 expression levels (FIG. 2C), revealing a correlation between Src-mediated STAT5 phosphorylation and PRL-3 expression.

STAT5 is a Potent Transcriptional Regulator of PRL-3 Expression

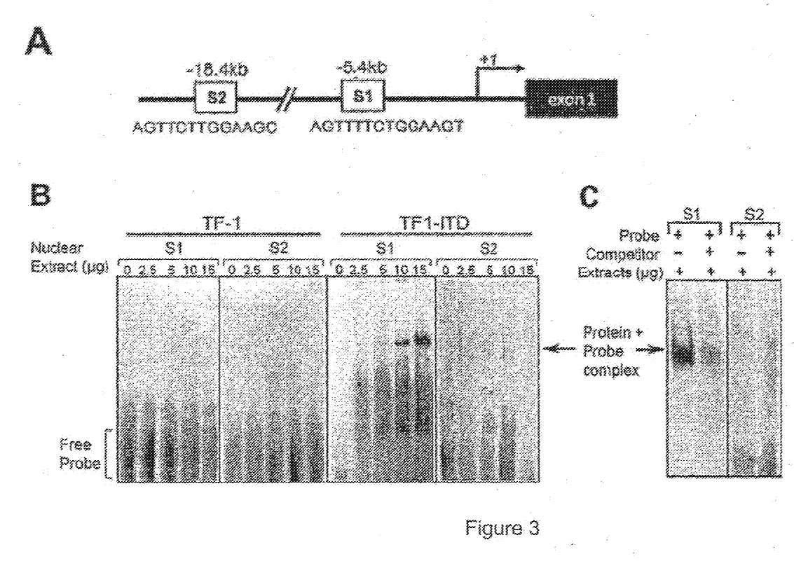

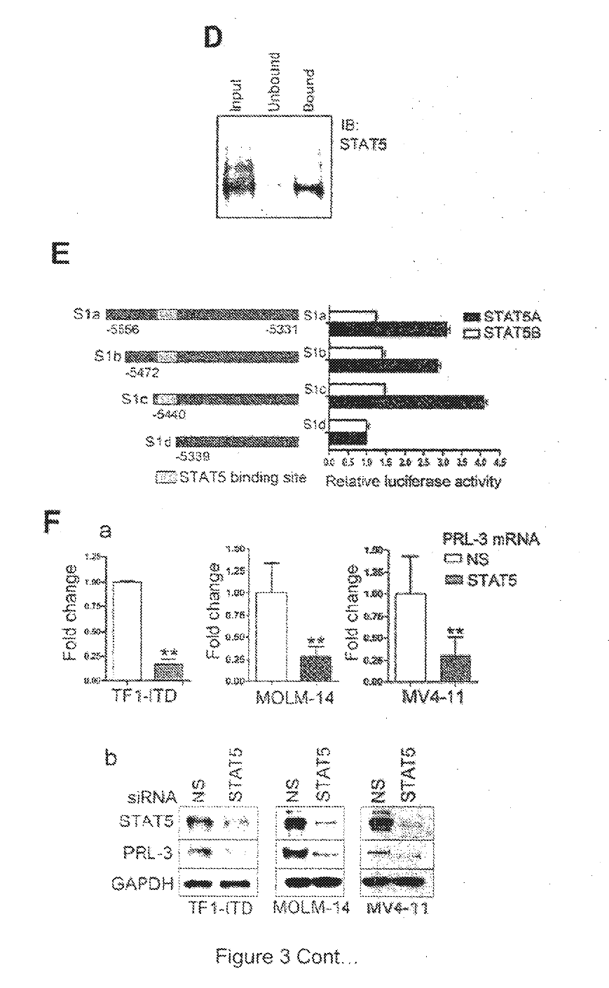

To understand how PRL-3 could be upregulated, the human PRL-3 promoter region was analysed by the Transcription Factor Database (TRANSFAC) to predict possible transcription factor binding sites (Wingender et al, 1996). The TRANSFAC program identified a number of putative transcription factors binding sites at the upstream promoter region of PRL-3, including two STAT5 consensus binding sequence TTCN(3)GAA (Seidel et al, 1995) (FIG. 3A). To evaluate the role of STAT5 as a transcriptional regulator of PRL-3, we designed two biotinylated probes. S1 and S2, corresponding to these STAT5 binding sequences and performed gel mobility shift assay (EMSA) using nuclear extracts from either TF-1 (PRL-3 non-expressing) or TF1-ITD (PRL-3 expressing) cells (FIG. 1C, lanes 1, 4). Nuclear extracts from TF1-ITD cells exhibited a robust level of DNA binding activity specifically to probe S1 (−5.4 kb) but not to probe S2 (−18.4 kb) while nuclear extracts from parental TF-1 cells had no observable DNA binding activity with probe S1 or S2 (FIG. 3B). Unlabelled competing oligonucleotides containing the STAT5 binding sequence could efficiently displace the labelled probe during the binding shift assay (FIG. 3C). To further ensure the involvement of STAT5 in this protein/DNA complex, streptavidin-agarose pull-down assay was performed using biotinylated probe S1. Consistent with the EMSA result, western blot analysis with STAT5 antibody confirmed that STAT5 was the transcription factor binding to the probe S1 in the complex (FIG. 3D).

To further clarify the binding property of STAT5 to the upstream region of PRL-3 promoter, reporter assays were carried out using co-transfection of either STAT5A or STAT5B expression vector together with pGL3 luciferase vectors containing either the −5.4 kb upstream sequence of the PRL-3 promoter region or its sequential 5′-deletion constructs (S1a, S1b, S1c, and S1d) (FIG. 3E, left panel). Similar protein expression levels of transfected STAT5A or STAT5B were identified. In TF-1 cells, when STAT5A expression vector was co-transfected with reporter constructs S1a-c, luciferase activities were increased 3-4 fold relative to the Sid deletion construct, which lacked of STAT5 binding site (FIG. 3E, black columns). Interestingly, co-expression of STAT5B with the reporter constructs showed no significant increase in reporter activity (FIG. 3E, open columns), suggesting that this activation could be specific for STAT5A but not for STAT5B. To support the role of STAT5 as a key transcription regulator of PRL-3, STAT5 was depleted by siRNA knock-down approach in three AML cell lines; TF1-ITD, MOLM-14, and MV4-11. Silencing of STAT5 attenuated PRL-3 mRNA (FIG. 3F, a), consequently, decreased in PRL-3 protein expression levels (FIG. 3F, b). These results further enforced the positive regulation of STAT5 on PRL-3 expression.

Up-Regulation of PRL-3 Activates AP-1 Oncogenic Transcription Factor Through ERK and JNK Cascades

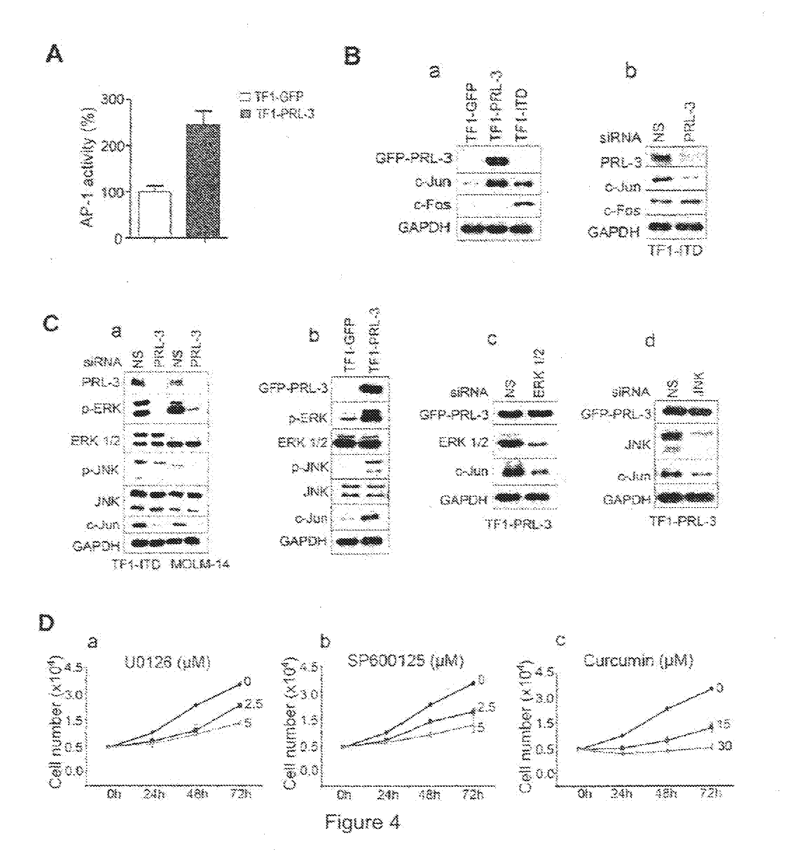

PRL-3 has been reported to play important roles in tumor development (Guo et al, 2006; Matsukawa et al, 2010). Thus, we investigated the molecular consequences of PRL-3 overexpression on various oncogenic transcription factors, such as AP-1, a well-known transcription factor driving tumorigenesis (Efert & Wagner, 2003). For this, we performed SEAP (Secreted Alkaline Phosphatase) assay with pAP1-SEAP vector, which contains the SEAP reporter gene under the control of AP-1 promoter, using TF1-PRL-3 (TF-1 cells overexpressing GFP-PRL-3) and TF1-GFP control cells. As shown in FIG. 4A, TF1-PRL-3 cells displayed a >2.5-fold increase in SEAP activity when compared to the TF1-GFP control cells, implying that PRL-3 could induce AP-1 expression. To investigate if this observation from TF-1 leukemia cell line is applicable to solid tumor cell lines, two colorectal carcinoma cell lines, DLD-1 and HCT116, were examined. Consistently, overexpression of PRL-3 led to a >6.5-fold and >2.5-fold increase in AP-1 activity in DLD-1 cells and HCT116 cells, respectively (FIG. 13).

To further, identify the activated AP-1 complex, we performed western blot analysis against c-Jun and c-Fos protein, the two key members of the AP-1 complex. Compared to TF1-GFP control cells, c-Jun was up-regulated in both TF1-PRL-3 and TF1-ITD cells while c-Fos was only detected in TF1-ITD but not in TF1-PRL-3 cells (FIG. 4B, a), indicating that PRL-3 preferentially stimulates c-Jun but not c-Fos. This result was verified by knock-down of PRL-3 in TF1-ITD cells, which showed that the loss of PRL-3 reduced c-Jun (but not c-Fos) expression (FIG. 4B, b). Since MAP kinases are actively involved in the regulation of AP-1 transcription factors (Zhang & Liu, 2002), we investigated whether induction of c-Jun might be a consequence of the activation of MAP kinases (MEK/ERK or JNK). Depletion of PRL-3 decreased phosphorylation of JNK and ERK, leading a subsequent loss of c-Jun phosphorylation in TF1-ITD and MOLM-14 cells (FIG. 4C, a). In addition, overexpression of PRL-3 induced ERK and JNK phosphorylation in TF1-PRL-3 cells compared to TF1-GFP cells (FIG. 4C, b). These results suggest that PRL-3 acts through ERK and/or JNK cascades to activate oncogenic c-Jun. To further confirm this, we knock-downed either ERK or JNK with respective siRNA in TF1-PRL-3 cells and the results showed that depletion of either ERK (FIG. 4C, c) or JNK (FIG. 4C, d) suppressed phosphorylation of c-Jun.

Since c-Jun is known to promote cell proliferation in various cancers (Hui et al, 2007; Zhang et al, 2007), we then investigated if activation of PRL-3-ERK/JNK-c-Jun pathway affect AML cell growth. TF1-PRL-3 cells treated with MEK-specific inhibitor (U0126, 5 μM) or JNK-specific inhibitor (SP600125, 5 μM) showed around 50% reduction in cell number compared to DMSO-treated control cells at 72 hr (FIG. 40, a-b). In addition, treatment with 15 μM curcumin, a general inhibitor of AP-1 family (Balasubramanian & Eckert, 2007; Wang et al, 2009), decreased cell number to ˜50% of DMSO-treated cells at 72 hr (FIG. 40, c).

PRL-3 Overexpression Promotes Cell Growth and Inhibits Apoptosis

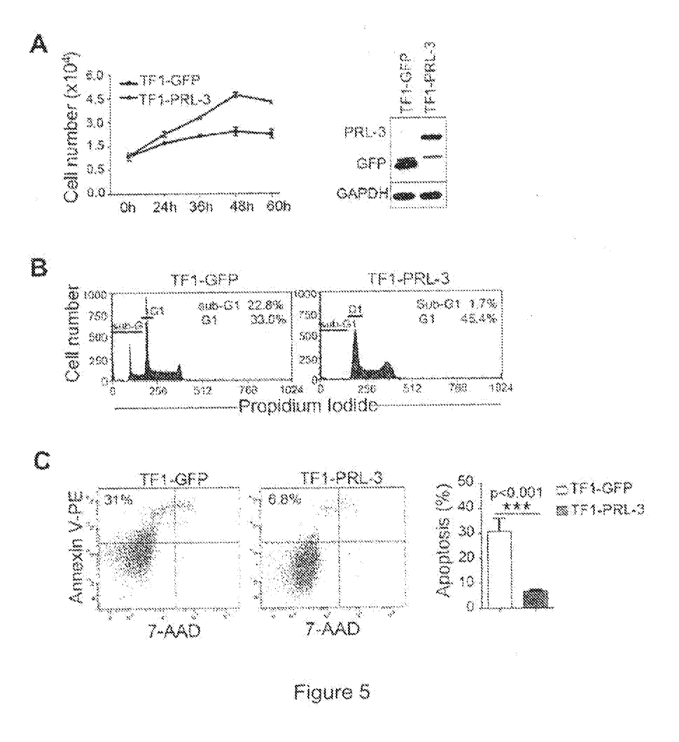

To investigate the biological outcomes of PRL-3 overexpression, the gain of PRL-3 function in TF-1 cells was examined. TF-1 is a cytokine dependent cell line required supplementation of cytokines such as IL-3 or GM-CSF in culture media to sustain cell growth and survival (Lin et al, 2007). Without cytokine, TF-1-GFP vector control cells grow poorly (FIG. 5A) and showed a 22.8% sub-G1 apoptotic population at 48 hr time point (FIG. 5B, left panel). However, TF-1 cells' overexpressing PRL-3 (TF1-PRL-3 cells) became cytokine independent in term of cell growth and cell number increased to around 2-fold of TF1-GFP control cells at the same time point (FIG. 5A). Furthermore, as presented in FIG. 5B, TF1-PRL-3 cells had a much smaller sub-G1 apoptotic population (1.7%, FIG. 5B, right panel) despite the lack of cytokine supplementation. To study anti-apoptotic activity of PRL-3 in the absence of cytokine supplementation, we performed Annexin-V and 7-aminoactinomycin D (7-AAD) staining followed by Fluorescence-activated cell sorting (FACS) analysis on TF1-GFP versus TF1-PRL-3 cell lines. More apoptotic population (31%) was observed in TF1-GFP cells than in TF1-PRL-3 cells (6.8%) after 48 hr culture without cytokine supplement (FIG. 5C), suggesting that PRL-3 might play an anti-apoptotic role and sustain the cell growth in TF1-PRL-3 AML cells.

PRL-3 Depletion Reduces Cell Growth

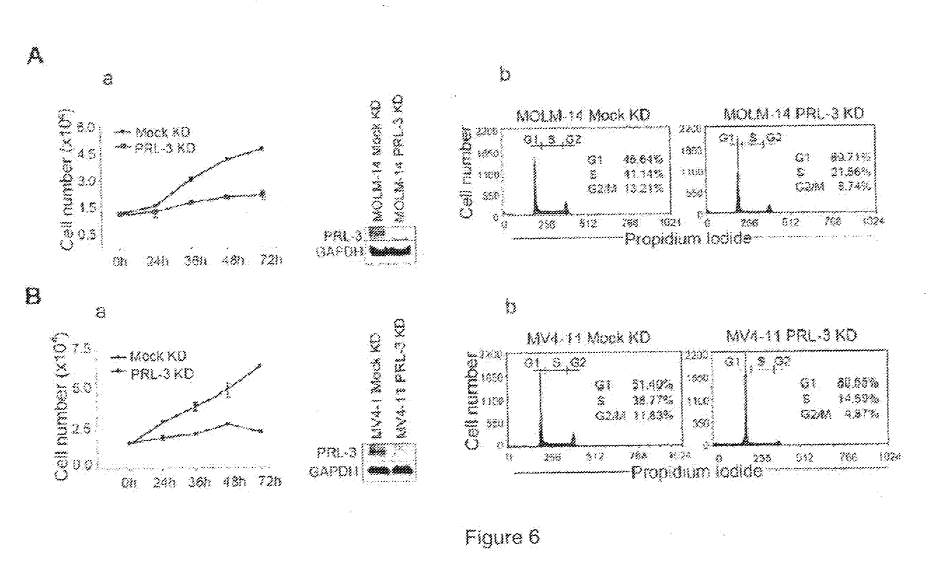

To investigate the loss of PRL-3 function in AML cell lines, we knocked down of PRL-3 in two cytokine independent cell lines (MOLM-14 and MV4-11) that highly express both endogenous FLT3-ITD and PRL-3 (FIG. 1C). After depletion of PRL-3, cell viability was assessed at various time points (FIG. 6A a, B a). Interestingly, silencing of PRL-3 by siRNA resulted in reduced cell number by ˜64.5% in MOLM-14 cells and ˜66.7% in MV4-11 cells compared to their mock knock-down cells at 48 hr. Furthermore, cell cycle analysis implied that the reduction in cell number in PRL-3-ablated cells correlated with increasing G1 and decreasing S phase populations (↑G1/S↓) in MOLM-14 and MV4-1.1 cell lines. The ratio of G1/S populations was 46.6%/41.1% in MOLM-14 mock knock-down cells, and became 69.7%/21.6% in MOLM-14 PRL-3 KD cells (FIG. 6A, b). Similarly, the ratio of G1/S populations in MV4-11 mock knock-down cells shifted from 51.4%/36.8% to 80.5%/14.6% in MV4-11 PRL-3 KD cells (FIG. 6B, b). Therefore, depletion of PRL-3 retards cells entering from G1 to S phase, implicating that PRL-3 may have roles in facilitating G1 to S phase transition to promote cell growth in both MOLM- and MV4-11 cell lines. However, depletion of PRL-3 did not affect on apoptosis as presented in cell cycle analysis (FIG. 6A b, B b). Results showed that there were no observable increments of sub-G1 cell population after PRL-3 knock-down. It was further confirmed by apoptosis analysis with Annexin-V and 7-AAD staining. FACS analysis showed that silencing of PRL-3 by siRNA did not show substantial increment of apoptotic population in both cell lines. These results imply that the role of PRL-3 is primarily in promoting G1-S transition in MOLM-14 and MV4-11 cytokine independent cells.

PRL-3 Antibody Shows Anti-Tumor Effect in Mouse Leukemia Model

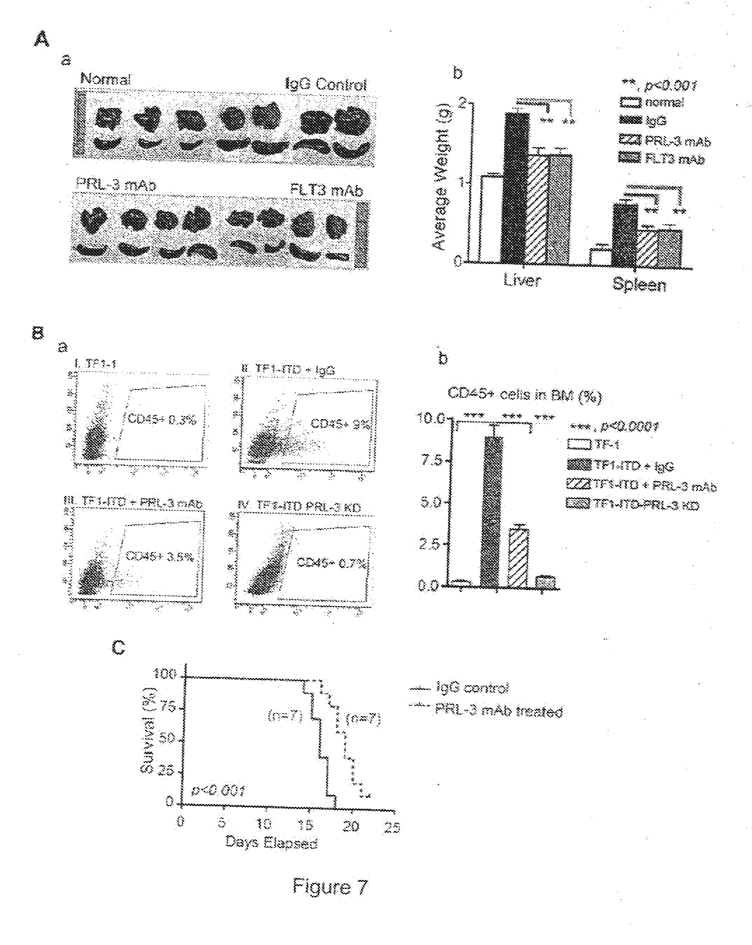

Our results thus far showed FLT3 and PRL-3 could synergistically drive AML cell growth. Given that clinical trials with FLT3 inhibitors have shown primary or secondary drug resistance (Wiernik, 2010) and the implication of PRL-3 in AML drug resistance (Zhou et al, 2011), we herein attempted to develop an alternative strategy by using PRL-3 antibody to target PRL-3 (an intracellular phosphatase) expressing AML cells. We and others have demonstrated the feasibility of antibody therapy against intracellular oncoproteins for anticancer immunotherapy (Dao et al, 2013; Guo et al, 2011; Guo et al. 2012). To ascertain if the in vitro role of PRL-3 correlated with FLT3-ITD-driven AML tumor burden in vivo, we developed a leukemia mouse model using the lateral tail vein injection of AML cells. PRL-3 monoclonal antibodies (mAb) (Li Jie et al., 2005) were subsequently used to target TF1-ITD AML cells which have elevated PRL-3 expression (FIG. 1C, lane 4). Balb/c nude mice injected with TF1-ITD cells were divided into three treatment groups: 1. IgG antibody sham-treatment (IgG-treated, n=11); 2. PRL-3 mAb (PRL-3 mAb-treated, n=11); or 3. FLT3 mAb (FLT3 mAb-treated, n=11). After bi-weekly administrations of IgG, PRL-3 or FLT3 mAbs over 12-14 days, PRL-3 mAb-treated mice showed a significant reduction of liver and spleen sizes (FIG. 7A, a), indicative of reduced tumor burden. Liver and spleen weights were decreased to 72.8% and 59.3% of untreated (IgG control) group, respectively (p<0.001. FIG. 7A, b). Notably, PRL-3 mAb treatment produced similar results to FLT3 mAb treatment (FIG. 7A a, b). Previously, FLT3 mAb treatment was demonstrated to have efficacy in an FLT3 leukemia mouse model (Li et al, 2004). The current results corroborate a role of PRL-3 in FLT3-ITD-driven AML progression and indicate a novel use of PRL-3 antibody therapy to treat PRL-3 positive AML patients, in addition to other PRL-3-positive cancer types previously investigated (Guo et al., 2012).

To understand the effect of PRL-3 mAb in reducing leukemia burden, we assessed the engraftment of these human leukemic cells in mouse bone marrow. Twenty balb/c nude mice were divided into 4 groups (FIG. 7B, I-IV, n=5/group): Mice injected with I. TF-1 cells; II. TF1-ITD cells+IgG (IgG-treated); III. TF1-ITD+PRL-3 mAb (PRL-3 mAb-treated); IV. TF1-ITD PRL-3 KD (no treated). An antibody against the CD45 human specific hematopoietic cell surface marker (hCD45) was used to distinguish and identify engrafted human leukemic cells from mouse host bone marrow cells by FACS analysis. Group I mice showed 0.3% of CD45-positive (hCD45+) cells engrafted in their bone marrows (FIG. 7B, a, panel I). In contrast, group II mice showed 9% of cells in their bone marrows were hCD45+(FIG. 7B, a, panel II). Group III mice showed only 3.5% of cells being hCD45+(FIG. 7B, a, panel III), indicating that PRL-3 mAb treatment could reduce TF1-ITD cell infiltration. Group IV mice showed the effects of PRL-3 silencing on leukemia development. We could detect only 0.7% of such hCD45+ cell in mouse bone marrow from group IV mice (FIG. 7B, a, panel IV), suggesting that knock-down of PRL-3 was more effective than PRL-3 mAb treatment with regards to cancer cells engraftment in mouse bone marrow. The statistical significance of leukemic infiltration in the different groups of mice is summarized in FIG. 7B (b) (p<0.001). Importantly, PRL-3 mAb therapy prolonged the survival rates for nude mice injected with TF1-ITD cells. Mice with a median survival of 19 days for PRL-3 mAb-treated but 16 days for control IgG-treated mice (FIG. 7C: p<0.001). Collectively, our results here demonstrate a significant benefit of PRL-3 immunotherapy in reducing FLT3-ITD AML cell engraftment in bone marrow and tumor burden, as well as in prolonging survival.

PRL-3 Expression in AML Patients Significantly Associates with a Shorter Survival

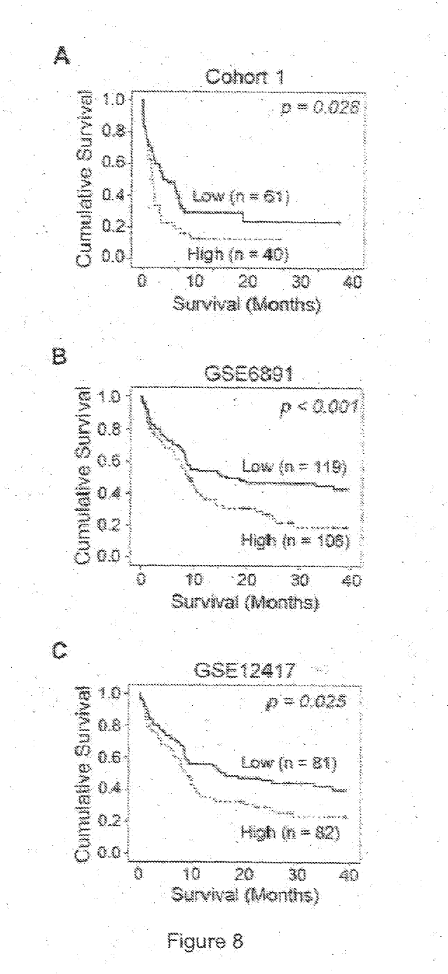

To evaluate the clinical relevance and importance of PRL-3 expression in AML, the correlation between PRL-3 gene expression and the overall survival in AML patients was analyzed using Cohort 1 (n=221) and a publicly available dataset GSE12417 (n=163) (Metzeler et al, 2008). By univariate Cox-regression analysis, high levels of PRL-3 expression were associated with a shorter survival in both Cohort 1 (HR=1.327, 95% CI=1.057-1.664, p=0.015) and GSE12417 cohort (HR=1.81, 95% CI=1.20-2.74, p=0.005). We noted that the prognostic value of PRL-3 was greater in AML patients with normal karyotype (n=101) (HR=1.576, 95% CI=1.151-2.156, p=0.005) than in AML patients with cytogenetic complications (n=120) (HR=1.298, 95% CI=0.927-1.818, p=0.129) in Cohort 1. We therefore focused on the relationship between PRL-3 and survival in such patients with normal karyotype by Kaplan-Meier survival analysis using the following 3 cohorts: 1. Cohort 1 (n=101), 2. GSE 6891 (n=227), and 3. GSE12417 (n=163). In Cohort 1, high levels of PRL-3 expression were significantly associated with a shorter survival (mean survival time=26 months, 95% CI=13-40 months) compared to patients with low PRL-3 expression levels (mean survival time=60 months, 95% CI=39-81 months) in patients with normal karyotype (log-rank test, n=101, p=0.028; FIG. 8A). In concordance, Kaplan-Meier survival analysis of AML patients with normal karyotype in the other two independent cohorts, GSE 6891 and GSE12417, also revealed that a high level of PRL-3 mRNA expression was significantly associated with a shorter survival time (p<0.001 and p=0.025, respectively; FIG. 8B-C). Together, these results suggest that PRL-3 expression is associated with poorer overall survival in AML patients with a normal karyotype.

Multivariable Cox-Regression Analysis Reveals PRL-3 as an Independent Prognostic Marker

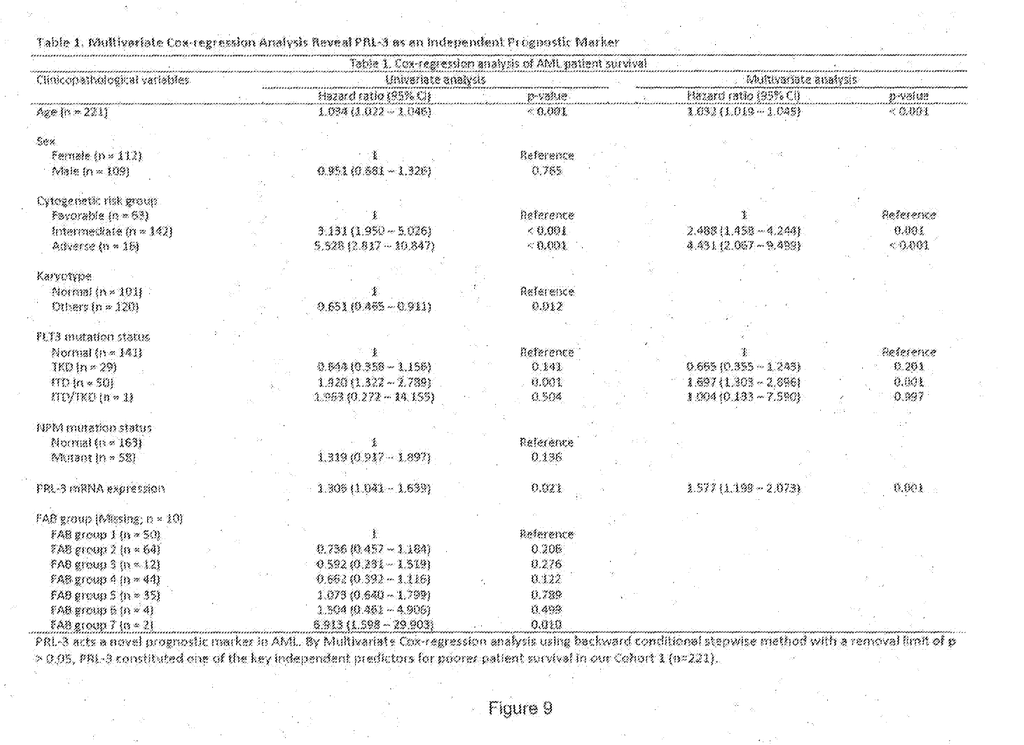

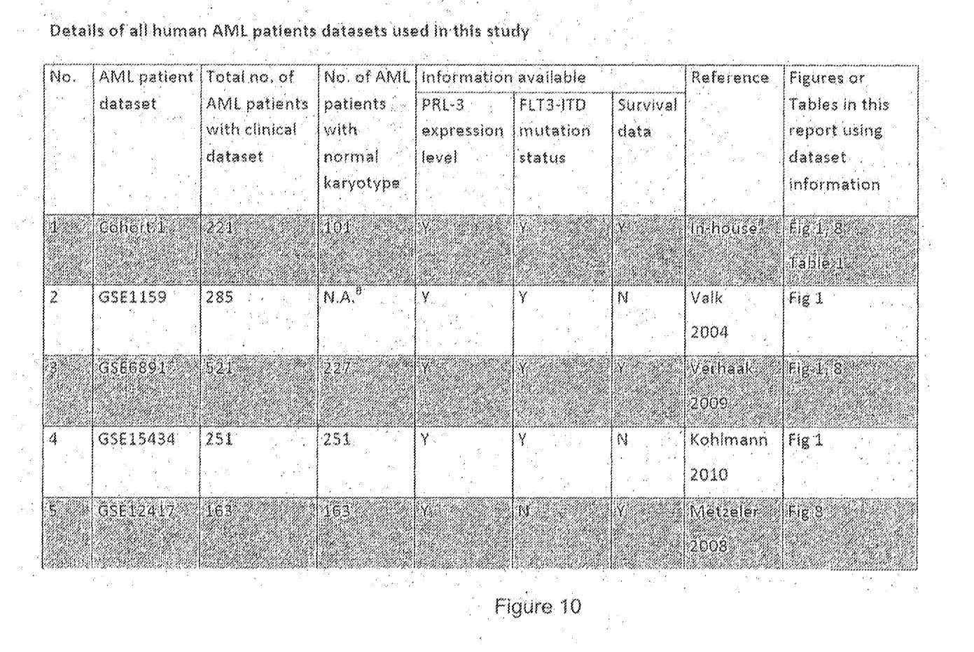

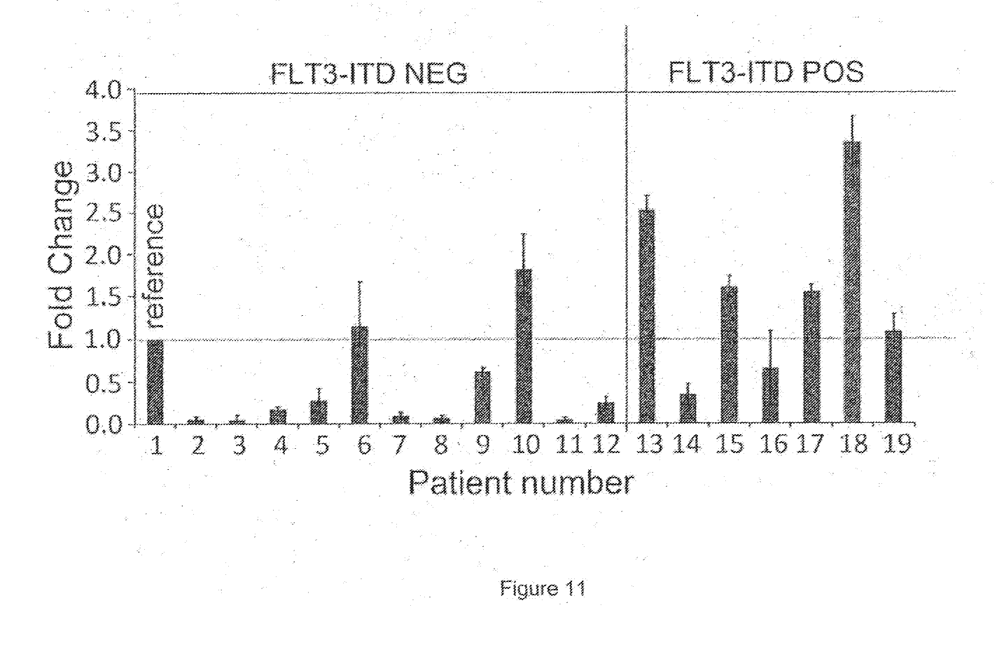

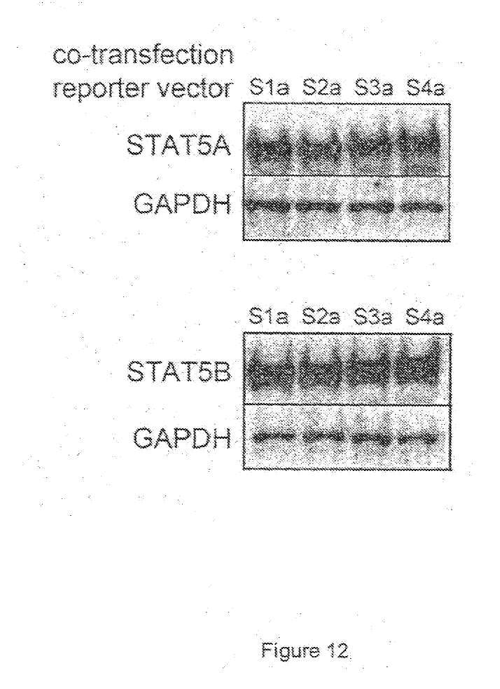

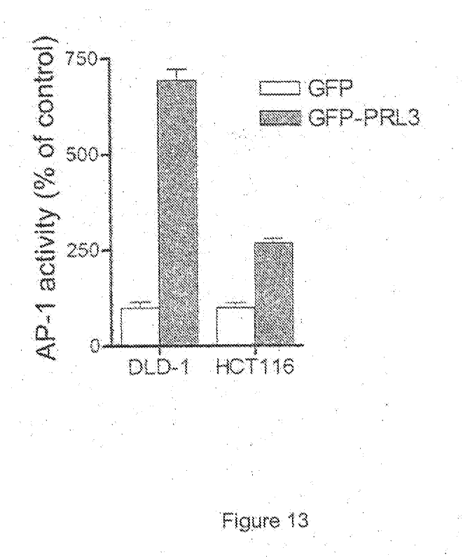

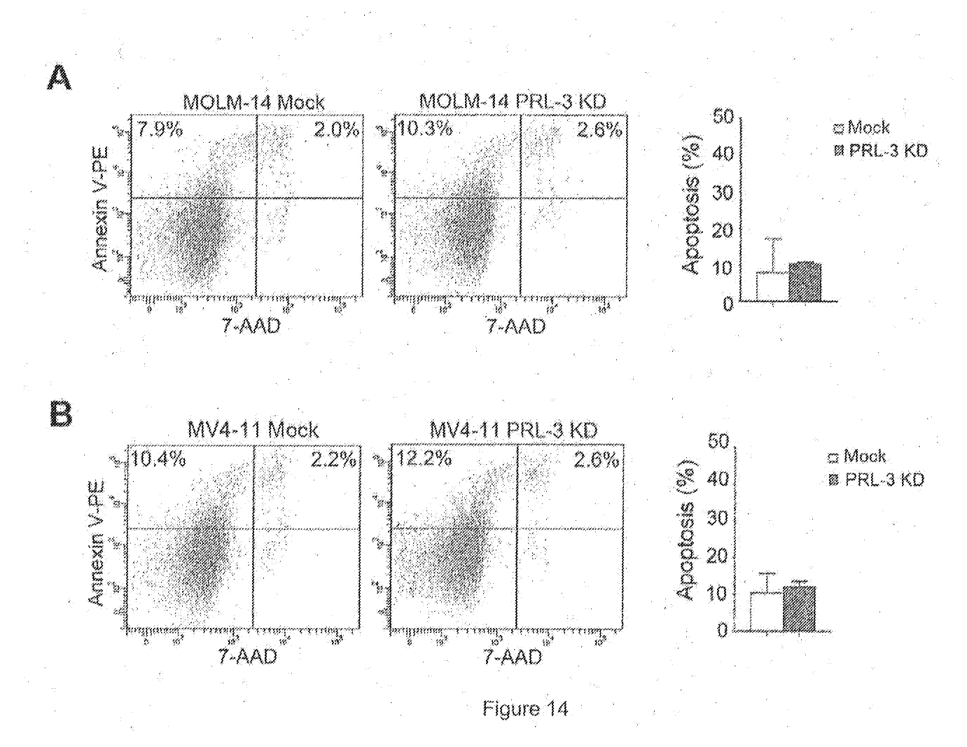

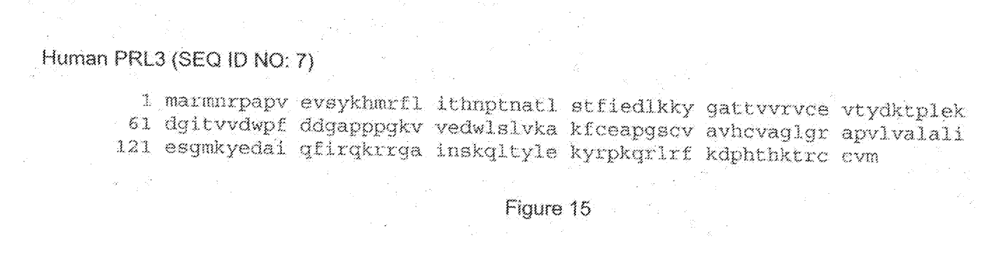

To evaluate whether PRL-3 is an independent prognostic marker for survival in AML patients, multivariable Cox-regression was performed in Cohort 1 (n=221) with parameters including sex, age, cytogenetic risk group, karyotype, FAB group. FLT3 mutation status, NPM mutation status, and PRL-3 mRNA expression (FIG. 9). Importantly, high PRL-3 mRNA expression (p=0.001, HR=1.577, 95% CI=1.199-2.073) was identified as an independent predictor for patient survival, in addition to age (p<0.001), cytogenetic risk group (Intermediate, p=0.001; Adverse, p<0.001) and FLT3-ITD mutation (p=0.001 in red). Consistently, examination of the GSE6891 dataset (n=521) (Verhaak et al., 2009) using multivariable analysis likewise demonstrated that high PRL-3 expression or FLT3-ITD mutation were independent predictors for patient survival. In that dataset, we found that only a high level of PRL-3 expression (HR=1.488, 95% CI=1.194-1.855, p<0.001) or FLT3-ITD mutation (HR=1.389, 95% CI=1.094-1.764, p=0.007) were shown to be independent predictors for patients survival. These consistent results from distinct datasets collectively indicate that a high level expression of PRL-3 is associated with poor survival, and highlight PRL-3 expression levels as an important and novel prognostic marker independent of other known clinically relevant prognostic markers for AML patients.

### DISCUSSION

In this study, we presented three major findings: 1) the molecular mechanism of PRL-3 overexpression in promoting AML cell growth in vitro, 2) novel approach of using PRL-3 antibody as unconventional therapies to target PRL-3 expressing AML cells for reducing tumor burden in animal model, and 3) a clinical relationship between high PRL-3 expression and poorer survival in AML patients. Collectively, our findings suggest that PRL-3 could be a putative novel therapeutic target and a prognostic marker to predict poorer survival for AML patients with FLT3-ITD mutations.

FLT3 and its mutants have received much attention as therapeutic drug targets, due to their prominent roles in cell proliferation and differentiation of myeloblasts (Levis & Small, 2003). So far, several FLT3 selective inhibitors have been developed and examined in AML patients as single agents or in combination with chemotherapy (Wiernik, 2010). However, recent clinical trials with FLT3 inhibitors showed primary or secondary drug resistance and differential clinical outcomes (Weisberg et al, 2009). Thus, the discovery of critical downstream target genes of FLT3 mutation will be important for improved therapies. Herein, the demonstration of PRL-3 as a putative novel target for AML therapy is a timely and an important endeavour. Currently, only a handful of studies have addressed, a possible link between PRL-3 expression and leukemia (Fagerli et at, 2008; Zhou et al, 2011). Here we report that AML cells and patient samples with FLT3-ITD mutations have a high incidence of PRL-3 overexpression, an observation supported by the analysis of four separate AML patient cohorts in a total of 1158 patients. PRL-3 is shown to be a downstream target of FLT3-ITD mutation, with a FLT3-Src-STAT5 pathway regulating PRL-3 mRNA expression. Importantly, PRL-3 upregulation by FLT3-ITD mutations associated with cancer progression, a phenomenon potentially explained by the PRL-3-induced activation of oncogenic transcription factor c-Jun/AP-1. c-Jun is overexpressed in AML patients and contributes to a block in granulocyte differentiation and development of AML (Pulikkan et al, 2010; Rangatia et al, 2003), thus implicating an important role of AP-1 activation by PRL-3 in tumor development. In addition, treatment with MEK/JNK inhibitors (U0126, SP600125) or AP-1 inhibitor (curcumin) resulted in a decrease in PRL-3 driven-cell growth, suggesting that PRL-3 function is dependent on MEK/ERK and/or JNK signalling. Several reports have shown that PRL-3 could activate ERK through regulation of Rho family GTPase (Fiordalisi J J et al, 2006, Ming J et al. 2009) or integrin β (Peng et al, 2009) in various solid cancer cells, but the detailed molecular mechanisms are not fully answered yet. In addition, it has been recently reported that PRLs (PRL-1, PRL-2, and PRL-3) can promote AP-1 activity and increase cell proliferation in non-small cell lung cancer cells (Hwang et al, 2011). Consistently, we demonstrate that PRL-3 played oncogenic roles in AML cell growth by promoting G1 to S phase transition in cell cycle as well as anti-apoptosis. More importantly, we showed PRL-3 up-regulation could contribute to AML progression, particularly in patients with normal karyotype, suggesting that PRL-3 was a viable therapeutic target for this group of patients, whose clinical outcomes to conventional therapies are highly heterogeneous (Baldus & Bullinger, 2008; Gaidzik & Dohner, 2008; Small, 2006). Moreover multivariable analysis validated PRL-3 expression as an independent prognostic marker in two distinct datasets (Cohort 1, GSE6891; FIG. 9). These results suggest that PRL-3 is a useful prognostic marker and a therapeutic target in AML patients.

Lastly, we demonstrated an unconventional antibody therapy approach to target intracellular PRL-3 oncoprotein for anti-AML therapy in mice (FIG. 7). Antibodies are traditionally used to target extracellular (surface) proteins and have never been used to target intracellular proteins because antibodies are generally believed to be too large (˜150 kDa) to enter cells, leaving a large intracellular treasure of potential cancer-specific therapeutic targets untapped in terms of antibody therapy or vaccination. The possible mechanisms for how antibodies could target intracellular oncoproteins for anti-cancer were proposed in recent review articles (Ferrone, 2011; Guo at al, 2011; Guo et al, 2008; Hong & Zeng, 2012). Herein, this untraditional approach was further evaluated by performing PRL-3 mAb therapy in mice carrying tumors formed by TF1-JTD cells expressed both FLT3-ITD and PRL-3 proteins. Compared to control IgG-treated mice, mice treated with FLT3 mAb (targeting extracellular FLT3 receptor), or treated with PRL-3 mAb (targeting intracellular PRL-3) showed reduction in the sizes of spleen and liver, two enlarged organs commonly used for indicator of leukaemia burden. This result suggests a potential value of PRL-3 antibody therapy for AML patients associated with PRL-3 overexpression. Since FLT3 inhibition both alone and in combination with standard chemotherapy have proven clinical limitations, PRL-3 antibody therapy might provide a viable alternative treatment for AML patients with the FLT3-ITD mutation associated with PRL-3 overexpression. Such an antibody treatment might be particularly useful and specific to AML patients as leukemia cells are easily accessible and are in direct contact with antibodies in their circulating system. The prospect of new therapeutic avenues by targeting PRL-3 in AML patients should be further explored.

### REFERENCES

Al-Aidaroos A Q, Zeng Q (2010) J Cell Biochem 111: 1087-1098
Balasubramanian S, Eckert R L (2007) J Biol Chem 282: 86707-6715
Baldus C D, Bullinger L (2008) Semin Oncol 35: 356-364
Bessette D C. Qlu D, Pallen C J (2008) Cancer Metastasis Rev 27: 231-252
Blake R A, Broome M A. Liu X; Wu J, Gishizky M, Sun L, Courtneidge S A (2000) Mol Cell Biol 20: 9018-9027
Dao T, Yan S, Veomett N, Pankov D. Zhou L, Korontsvit T. Scott A, Whitten J, Maslak P,
Casey E, Tan T. Liu H, Zakhaleva V, Curcio M, Doubrovina E, O'Reilly R J. Liu C,
Scheinberg D A (2013) Science Translational Medicine 5: 176ra133
Eferl R, Wagner E F (2003) Nat Rev Cancer 3: 859-868
Fagerli U-M, Holt R U, Holien T, Vaatsveen T K, Zhan F, Egeberg K W, Barlogie B, Waage A, Aarset H, Dai H Y, Shaughnessy J D, Sundan A. B [symbol] rset M (2008) Blood 111: 806-815
Ferrone S (2011) Sci Transl Med 3: 99ps38
Gaidzik V, Dohner K (2008) Semin Oncol 35: 346-355
Guo K, Li J, Tang J P, Tan C P, Hong C W, Al-Aidaroos A Q, Varghese L, Huang C; Zeng Q (2011) Sci Trans Med 3: 99ra85
Guo K, Li J, Wang H. Osato M, Tang J P, Quah S Y, Gan S Q, Zeng Q (2006) Cancer Res 66: 9625-9635
Guo K. Tang J P, Jie L, Al-Aidaroos A Q, Hong C W, Tan C P, Park J E, Varghese L, Feng Z, Zhou J, Chng W J, Zeng Q (2012) Oncotarget 3: 158-171
Guo K. Tang J P. Tan C P. Wang H; Zeng Q (2008) Cancer Biol Ther 7: 750-757
Hong C W, Zeng Q (2012) Cancer Res 72: 3715-3719
Hui L, Bakiri L, Mairhorfer A, Schweifer N, Haslinger C. Kenner L, Komnenovic V. Scheuch H, Beug H, Wagner E F (2007) Nat Genet 39:741-749
Hwang J J, Min S H, Sin K H, Heo Y S, Kim K D, Yoo O J, Lee S H (2011) Oncol Rep 27: 535-540
Kim K T, Baird K, Ahn J Y. Meltzer P, Lilly M, Levis M, Small D (2005) Blood 105: 1759-1767
Kohlmann A, Bullinger L, Thiede C, Schaich M, Schnittger S, Dohner K, Dugas M, Klein H U, Dohner H, Ehninger G, Haferlach T (2010) Leukemia 24: 1216-1220 Levis M, Small D (2003) FLT3: Leukemia 17: 1738-1752
Li J, Guo K, Koh V W C, Tang J P, Gan B Q, Shi H. Li H X, Zeng Q (2005) Clinical Cancer Research 11: 2195-2204
Li Y, Li H, Wang M-N. Lu D, Bassi R, Wu Y, Zhang H. Balderes P, Ludwig D L, Pytowski B, Kussie P, Piloto O. Small D, Bohlen P. Witte L, Zhu Z, Hicklin D J (2004) Blood 104: 1137-1144
Liang F, Liang J, Wang W Q, Sun J P, Udho E, Zhang Z Y (2007) J Biol Chem 282: 5413-5419
Lin K R. Lee S F, Hung C M, Li C L, Yang-Yen H F, Yen J J (2007) The Journal of biological chemistry 282: 21962-21972
Lowenberg B, Downing J R, Burnett A (1999) N Engl J Med 341: 1051-1062
Matsukawa Y. Semba S, Kato H, Koma Y, Yanagihara K, Yokozaki H (2010) Pathobiology 77: 155-162
Metzeler K H, Hummel M, Bloomfield C D, Spiekermann K, Braess J, Sauertand M C, Heinecke A, Radmacher M, Marcucci G, Whitman S P, Maharry K, Paschka P. Larson R A, Berdel W E, Buchner T, Wormann B, Mansmann U, Hiddemann W, Bohlander S K, Buske C (2008) Blood 112: 4193-4201
Mizuki M, Fenski R, Halfter H, Matsumura I, Schmidt R, Muller C, Gruning W, Kratz-Albers K, Serve S, Steur C, Buchner T, Kienast J, Kanakura Y, Berdel W E, Serve H (2000) Blood 96: 3907-3914
Nakao M, Yokota S, Iwai T, Kaneko H, Horiike S, Kashima K. Sonoda Y, Fujimoto T, Misawa S (1996) Leukemia 10: 1911-1918
Nam J-S, Ino Y, Sakamoto M, Hirohashi S (2002) Clinical Cancer Research 8: 2430-2436 Odgerel T, Kikuchi J, Wada T, Shimizu R, Futaki K, Kano Y. Furukawa Y (2007) Oncogene 27: 3102-3110
Pulikkan J A, Dengler V, Peer Zada A A, Kawasaki A, Geletu M, Pasalic Z, Bohlander S K. Ryo A, Tenen D G, Behre G (2010) Leukemia 24: 914-923
Quentmeier H, Reinherdt J. Zaborski M, Drexler H G (2003) Leukemia 17: 120-124
Rangatia J, Vangala R K, Singh S M, Peer Zada A A, Elsasser A, Kohlmann A, Haferlach T, Tenen D G, Hiddemann W. Behre G (2003) Oncogene 22: 4760-4764
Ren T, Jiang B, Xing X, Dong B, Peng L, Meng L, Xu H, Shou C (2009) Pathol Oncol Res 15: 555-560
Robinson U, Xue J, Corey S J (2005) Exp Hematol 33: 469-479
Rockova V, Abbas S, Wouters B J, Erpelinck C A J, Beverloo H B, Delwel R, van Putten W L J, Löwenberg B, Valk P J M (2011) Blood 118: 1069-1076
Seidel H M, Milocco L H, Lamb P, Darnell J E, Stein R B. Rosen J (1995) Proceedings of the National Academy of Sciences 92: 3041-3045
Small D (2006) Hematology 2006: 178-184.
Smith B D, Levis M, Beran M, Giles F, Kantarjian H, Berg K, Murphy K M, Dauses T, Allebach J. Small D (2004) Blood 103: 3669-3676
Spiekermann K, Bagrintseva K, Schwab R, Schmieja K, Hiddemann W (2003) Clin Cancer Res 9: 2140-2150
Sternberg D W, Licht J D (2005) Curr Opin Hematol 12: 7-13
Valk P J, Verhaak R G, Beijen M A, Erpelinck C A, Barjesteh van Waalwijk van Doom-Khosrovant S, Boer J M, Beverloo H B, Moorhouse M J, van der Spek P J, Lowenberg B, Delwel R (2004) N Engl J Med 350: 1617-1628
Verhaak R G, Wouters B J, Erpelinck C A, Abbas S, Beverloo H B, Lugthart S, Lowenberg B, Deiwel R, Valk P J (2009) Haematologica 94: 131-134
Wang L, Shen Y, Song R, Sun Y, Xu J, Xu Q (2009) Mol Pharmacol 76: 1238-1245
Weisberg E, Barrett R, Liu Q, Stone R. Gray N, Griffin J D (2009) Drug Resist Updat 12: 81-89
Weisberg E, Sattler M, Ray A, Griffin J D (2010) Oncogene 29: 5120-5134
Wiernik P H (2010) Clin Adv Hematol Oncol 8: 429-436, 444
Wingender E, Dietze P, Karas H, Knüppel R (1996) Nucleic Acids Research 24: 238-241
Zeng Q, Hong W, Tan Y H (1998) Biochem Biophys Res Commun 244: 421-427
Zhang W, Liu H T (2002) Cell Res 12: 9-18
Zhang Y, Pu X, Shi M. Chen L, Song Y, Qian L, Yuan G, Zhang H, Yu M, Hu M, Shen B, Guo N (2007) BMC Cancer 7:145
Zhou J, Bi C, Chng W. Cheong L, Uu S. Mahara S, Tay K, Zeng Q, Li J, Guo K, Tan C P B. Yu H, Albert D H, Chen C (2011) PLoS One 6: e19798

1-31. (canceled)

32. A method of detecting PRL3 in a patient with a FLT3-ITD positive cancer, the method comprising the steps of:
a) obtaining a sample from a patient with a FLT3-ITD positive cancer; and
b) detecting whether PRL3 is present in the sample by contacting the sample with an anti-PRL3 antibody and detecting binding between PRL3 and the antibody.

33. The method of claim 32, wherein the cancer is a leukemia.

34. The method of claim 33, wherein the leukemia is acute or chronic myeloid leukemia.

35. The method of claim 32, wherein the sample is a sample of bodily fluid.

36. The method of claim 32, wherein the sample is a bone marrow sample.

37. The method of claim 32, wherein the sample has a normal karyotype.

38. A method of treating a cancer patient, the method comprising the steps of:
a) detecting whether PRL3 is present in a sample obtained from the patient by contacting the sample with an anti-PRL3 antibody and detecting binding between PRL3 and the antibody; and
b) administering a therapeutically effective amount of an anti-PRL3 antibody to the patient if the level of PRL3 detected in the sample is elevated relative to a control.

39. The method of claim 38, wherein the control is a sample obtained from a non-cancerous tissue of the patient.

40. The method of claim 38, wherein the patient has previously undergone FLT3 inhibition therapy.

41. The method of claim 38, wherein the cancer is a FLT3-ITD positive cancer

42. The method of claim 38, wherein the cancer is a leukemia.

43. The method of claim 42, wherein the leukemia is acute or chronic myeloid leukemia.

44. The method of claim 38, wherein the sample is a sample of bodily fluid.

45. The method of claim 38, wherein the sample is a bone marrow sample.

46. The method of claim 38, wherein the sample has a normal karyotype.

47. A method of treating a cancer patient, the method comprising administering a therapeutically effective amount of an anti-PRL3 antibody to the patient, wherein a sample obtained from the patient has previously been determined to include a level of PRL3 that is elevated relative to a control.

48. The method of claim 47, wherein the control is a sample obtained from a non-cancerous tissue of the patient.

49. The method of claim 47, wherein the patient has previously undergone FLT3 inhibition therapy.

50. The method of claim 47, wherein the cancer is a FLT3-ITD positive cancer

51. The method of claim 47, wherein the cancer is a leukemia.

52. The method of claim 51, wherein the leukemia is acute or chronic myeloid leukemia.

53. The method of claim 47, wherein the sample is a sample of bodily fluid.

54. The method of claim 47, wherein the sample is a bone marrow sample.

55. The method of claim 47, wherein the sample has a normal karyotype.
